SEQUENCE LISTING (162 sequences)

SEQ ID NO: 1 - DNA, 31 bp, synthetic construct
  source 1..31: mol_type = other DNA
tataactagtatggtgagcaagggcgaggag

SEQ ID NO: 2 - DNA, 41 bp, synthetic construct
  source 1..41: mol_type = other DNA
tatacgtctcatctacagattcttccggcgtgtataccttc

SEQ ID NO: 3 - DNA, 53 bp, synthetic construct
  source 1..53: mol_type = other DNA
tatacgtctcatagagatccccggtcgccaccgtgagcaagggcgaggagctg

SEQ ID NO: 4 - RNA, 151 nt, synthetic construct
  source 1..151: mol_type = other RNA
actacagttgctccgatatttaggctacgtcaataggcactaacttattggcgctggtgaacggacttcctctcgagtaccagaagatgactacaaaactcctttccattgcgagtatcggagtctggctcagtttggccagggaggcact

SEQ ID NO: 5 - DNA, 508 bp, unidentified
  source 1..508: mol_type = unassigned DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 6 - DNA, 240 bp, unidentified
  source 1..240: mol_type = unassigned DNA
gagggcctatttccatgattccttcatatttgcatatacgatacaaggctgttagagagataattagaattaatttgactgtaaacacaaagatattagtacaaaatacgtgacgtagaaagtaataatttcttgggtatttgcagtttttaaaattatgttttaaaatggactatcatatgcttaccgtaacttgaaagtatttcgatttcttggctttatatatcttgtggaaaggac

SEQ ID NO: 7 - DNA, 708 bp, synthetic construct
  source 1..708: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 8 - DNA, 99 bp, synthetic construct
  source 1..99: mol_type = other DNA
ctgcagggcggaggaggcagcggcggaggaggcagcggcggaggaggcagcagaaggtatacacgccggaagaatctgtagagatccccggtcgccacc

SEQ ID NO: 9 - DNA, 717 bp, synthetic construct
  source 1..717: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 10 - DNA, 47 bp, unidentified
  source 1..47: mol_type = unassigned DNA
ctgccatcagtcggcgtggactgtagaaccatgccgactgatggcag

SEQ ID NO: 11 - DNA, 26 bp, unidentified
  source 1..26: mol_type = unassigned DNA
ctgccatcagtcggcgtggactgtag

SEQ ID NO: 12 - DNA, 21 bp, unidentified
  source 1..21: mol_type = unassigned DNA
aaccatgccgactgatggcag

SEQ ID NO: 13 - RNA, 151 nt, Homo sapiens
  source 1..151: mol_type = unassigned RNA
gatgtaggctttattttaagcagtaatgggttacttctgaaacatcacttgtttgcttaattctacacagtacttagattttttttactttccagtcccaggaagtgtcaatgtttgttgagtggaatattgaaaatgtaggcagcaactg

SEQ ID NO: 14 - RNA, 151 nt, synthetic construct
  source 1..151: mol_type = other RNA
cagttgctgcctacattttcaatattccactcaacaaacattgacacttcctgggactggaaagtaaaaaaaatccaagtactgtgtagaattaagcaaacaagtgatgtttcagaagtaacccattactgcttaaaataaagcctacatc

SEQ ID NO: 15 - RNA, 143 nt, synthetic construct
  source 1..143: mol_type = other RNA
cagttgctgcctacattttcaatattccactcaacaaacattgacacttcctgggactggaaagaaaaaaaatccaagtactgtgtagaataagcaaacaaggatgtcagaagaacccatactgctaaaataaagcctacatc

SEQ ID NO: 16 - RNA, 151 nt, Homo sapiens
  source 1..151: mol_type = unassigned RNA
ctacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggccc

SEQ ID NO: 17 - RNA, 151 nt, synthetic construct
  source 1..151: mol_type = other RNA
gctggtgcaggggccacggggggagcagcctctggcattctgggagcttcatctggacctgggtcttcagtgaaccattgttcaatatcgtccggggacagcatcaaatcatccattgcttgggacggcaagggggacagaacgttgtttt

SEQ ID NO: 18 - RNA, 151 nt, synthetic construct
  source 1..151: mol_type = other RNA
gctggtgcaggggccacggggggagcagcctctggcattctgggagcttcatctggacctgggtcttcagtgaaccattgttcaagatcgtccggggacagcatcaaatcatccattgcttgggacggcaagggggacagaacgttgtttt

SEQ ID NO: 19 - RNA, 151 nt, synthetic construct
  source 1..151: mol_type = other RNA
gctggtgcaggggccacggggggagcagcctctggcagtcggggagcttcatctggacctgggtcttcagtgaaccattgttcaagatcgtccggggacagcatcaaatcatccagtgcttgggacggcaagggggacagaacgttgtttt

SEQ ID NO: 20 - RNA, 150 nt, synthetic construct
  source 1..150: mol_type = other RNA
gctggtgcaggggccacggggggagcagcctctggcattctgggagcttcatctggacctgggtcttcagtgaaccattgttcaaatcgtccggggacagcatcaaatcatccattgcttgggacggcaagggggacagaacgttgtttt

SEQ ID NO: 21 - RNA, 147 nt, synthetic construct
  source 1..147: mol_type = other RNA
gctggtgcaggggccacggggggagcagcctctggcatcgggagcttcatctggacctgggtcttcagtgaaccattgttcaaatcgtccggggacagcatcaaatcatccatgcttgggacggcaagggggacagaacgttgtttt

SEQ ID NO: 22 - DNA, 50 bp, unidentified
  source 1..50: mol_type = unassigned DNA
aaaaacaaaaaacaaaaaaaacaaaaaaaaaaccaaaaaaacaaaacaca

SEQ ID NO: 23 - RNA, 151 nt, Mus musculus
  source 1..151: mol_type = unassigned RNA
aggaagccagatgctaggtatgagagagccaacagcctcagccctctgcttggcttatagatggagaacaactctaggcagaggtctcaaaggctggggctgtgttggacagcaatcatacagtgggtgtcctggccagcacccatcaccc

SEQ ID NO: 24 - RNA, 151 nt, synthetic construct
  source 1..151: mol_type = other RNA
gggtgatgggtgctggccaggacacccactgtatgattgctgtccaacacagccccagcctttgagacctctgcccagagttgttctccatctataagccaagcagagggctgaggctgttggctctctcatacctagcatctggcttcct

SEQ ID NO: 25 - RNA, 151 nt, Mus musculus
  source 1..151: mol_type = unassigned RNA
cccacgtgcagttgctgcgaaagccagtactcacagtcatggggctcatggccctgttggatggagaacaactctaggcagaggtctcaaaggctggggctgtgttggacagcaatcatacagtgggtgtcctggccagcacccatcaccc

SEQ ID NO: 26 - RNA, 151 nt, synthetic construct; identical to SEQ ID NO: 24
  source 1..151: mol_type = other RNA

SEQ ID NO: 27 - RNA, 171 nt, Homo sapiens
  source 1..171: mol_type = unassigned RNA
ggccaccatgcccagctgctgcctacattttcaatattctactcaacaaacattgagacttcctgggtctggaaagaaagaaatccaagtattatgtagacttaagcaaacaagtgatgtttcagaagtaacccattactgcttaaaataaagcctacatcaacactctaa

SEQ ID NO: 28 - RNA, 171 nt, Homo sapiens
  source 1..171: mol_type = unassigned RNA
catggaacccaaagggaactgcagcgagagcacaaagattctaggatactgcgagcaaatggggtggaggggtgctctcctgagccacagaaggaatggtctggtggttaagataaaacacaagtcaaacttattcgagttgtccacagtcagcaatggtgatcttcttgc

SEQ ID NO: 29 - RNA, 171 nt, Homo sapiens
  source 1..171: mol_type = unassigned RNA
taaaagaaaataataaaatataaaaatatattgcaaaggaggtgatgagagggcagcgtgggcacagtgcaaccccagctcgccgaccgccagtggaggtgcagcccagtggggctcagcacaggctcatggatttccaggggctggaggccctcttggcacagggacctc

SEQ ID NO: 30 - RNA, 170 nt, synthetic construct
  source 1..170: mol_type = other RNA
catggaacccaaagggaactgcagcgagagcacaaagattctaggatactgcgagcaaatggggtggagggggctctcctgagccacagaaggaatggtctggtggttaagataaaacacaagtcaaacttattcgagttgtccacagtcagcaatggtgatcttcttgc

SEQ ID NO: 31 - RNA, 170 nt, synthetic construct
  source 1..170: mol_type = other RNA
catggaacccaaagggaactgcagcgagagcacaaagattctaggatactgcgagcaaatggggtggaggggtgctctccgagccacagaaggaatggtctggtggttaagataaaacacaagtcaaacttattcgagttgtccacagtcagcaatggtgatcttcttgc

SEQ ID NO: 32 - RNA, 170 nt, synthetic construct
  source 1..170: mol_type = other RNA
catggaacccaaagggaactgcagcgagagcacaaagattctaggatactgcgagcaaatggggtggaggggtgctctcctgagccacagaaggaatggtctggtggtaagataaaacacaagtcaaacttattcgagttgtccacagtcagcaatggtgatcttcttgc

SEQ ID NO: 33 - RNA, 170 nt, synthetic construct
  source 1..170: mol_type = other RNA
catggaacccaaagggaactgcagcgagagcacaaagattctaggatactgcgagcaaatggggtggaggggtgctctcctgagccacagaaggaatggtctggtggttaagaaaaacacaagtcaaacttattcgagttgtccacagtcagcaatggtgatcttcttgc

SEQ ID NO: 34 - RNA, 169 nt, synthetic construct
  source 1..169: mol_type = other RNA
catggaacccaaagggaactgcagcgagagcacaaagattctaggatactgcgagcaaatggggtggaggggtgctctcctgagccacagaaggaatggtctggtggtaagaaaaacacaagtcaaacttattcgagttgtccacagtcagcaatggtgatcttcttgc

SEQ ID NO: 35 - RNA, 168 nt, synthetic construct
  source 1..168: mol_type = other RNA
catggaacccaaagggaactgcagcgagagcacaaagattctaggatactgcgagcaaatggggtggaggggtgctctccgagccacagaaggaatggtctggtggtaagaaaaacacaagtcaaacttattcgagttgtccacagtcagcaatggtgatcttcttgc

SEQ ID NO: 36 - RNA, 167 nt, synthetic construct
  source 1..167: mol_type = other RNA
catggaacccaaagggaactgcagcgagagcacaaagattctaggatactgcgagcaaatggggtggagggggctctccgagccacagaaggaatggtctggtggtaagaaaaacacaagtcaaacttattcgagttgtccacagtcagcaatggtgatcttcttgc

SEQ ID NO: 37 - RNA, 170 nt, synthetic construct
  source 1..170: mol_type = other RNA
taaaagaaaataataaaatataaaaatatattgcaaaggaggtgatgagagggcagcgtgggcacagtgcaaccccagctcgccgaccgccagtggagggcagcccagtggggctcagcacaggctcatggatttccaggggctggaggccctcttggcacagggacctc

SEQ ID NO: 38 - RNA, 170 nt, synthetic construct
  source 1..170: mol_type = other RNA
taaaagaaaataataaaatataaaaatatattgcaaaggaggtgatgagagggcagcgtgggcacagtgcaaccccagctcgccgaccgccagtggaggtgcagcccagtggggccagcacaggctcatggatttccaggggctggaggccctcttggcacagggacctc

SEQ ID NO: 39 - RNA, 170 nt, synthetic construct
  source 1..170: mol_type = other RNA
taaaagaaaataataaaatataaaaatatattgcaaaggaggtgatgagagggcagcgtgggcacagtgcaaccccagctcgccgaccgccagtggaggtgcagcccagtggggctcagcacaggccatggatttccaggggctggaggccctcttggcacagggacctc

SEQ ID NO: 40 - RNA, 170 nt, synthetic construct
  source 1..170: mol_type = other RNA
taaaagaaaataataaaatataaaaatatattgcaaaggaggtgatgagagggcagcgtgggcacagtgcaaccccagctcgccgaccgccagtggaggtgcagcccagtggggctcagcacaggctcatggatttccaggggctggaggccctctggcacagggacctc

SEQ ID NO: 41 - RNA, 169 nt, synthetic construct
  source 1..169: mol_type = other RNA
taaaagaaaataataaaatataaaaatatattgcaaaggaggtgatgagagggcagcgtgggcacagtgcaaccccagctcgccgaccgccagtggaggtgcagcccagtggggctcagcacaggccatggatttccaggggctggaggccctctggcacagggacctc

SEQ ID NO: 42 - RNA, 168 nt, synthetic construct
  source 1..168: mol_type = other RNA
taaaagaaaataataaaatataaaaatatattgcaaaggaggtgatgagagggcagcgtgggcacagtgcaaccccagctcgccgaccgccagtggaggtgcagcccagtggggccagcacaggccatggatttccaggggctggaggccctctggcacagggacctc

SEQ ID NO: 43 - RNA, 167 nt, synthetic construct
  source 1..167: mol_type = other RNA
taaaagaaaataataaaatataaaaatatattgcaaaggaggtgatgagagggcagcgtgggcacagtgcaaccccagctcgccgaccgccagtggagggcagcccagtggggccagcacaggccatggatttccaggggctggaggccctctggcacagggacctc

SEQ ID NO: 44 - RNA, 151 nt, Homo sapiens
  source 1..151: mol_type = unassigned RNA
actgaaggcggcggcggggccagaggctcagcggctcccaggcctgctgaaaatgactgaatataaacttgtggtagttggagctggtggcgtaggcaagagtgccttgacgatacagctaattcagaatcattttgtggacgaatatgat

SEQ ID NO: 45 - RNA, 151 nt, Homo sapiens
  source 1..151: mol_type = unassigned RNA
aatgcaggcctgtaaggtggagggttgaaccctgtttggattgcagagtgttactcagaattgggaaatccagctagcggcagtattctgtacagtagacacaagaattatgtacgccttttatcaaagacttaagagccaaaaagctttt

SEQ ID NO: 46 - RNA, 151 nt, Homo sapiens
  source 1..151: mol_type = unassigned RNA
ctgcggaggtccctttgagagctggttcctgttcattcacttcggaggatgggctgagatggtggcagagcaattactgatgtcggcagccgaaccccccacggccctgctgtggctcttggccttctactacggcccccgtgatgggagg

SEQ ID NO: 47 - RNA, 151 nt, Homo sapiens
  source 1..151: mol_type = unassigned RNA
tcagttgcgtaatggaaagtaaagccctgaactatcacactttaatcttccttcaaaaggtggtaaactatacctactgtccctcaagagaacacaagaagtgctttaagaggtattttaaaagttccgggggttttgtgaggtgtttgat

SEQ ID NO: 48 - DNA, 19 bp, synthetic construct
  source 1..19: mol_type = other DNA
cgcgatgctagagggctct

SEQ ID NO: 49 - DNA, 19 bp, synthetic construct
  source 1..19: mol_type = other DNA
cgcgatgctggagggctct

SEQ ID NO: 50 - DNA, 19 bp, synthetic construct
  source 1..19: mol_type = other DNA
cgcgatgctggagggctct

SEQ ID NO: 51 - RNA, 137 nt, synthetic construct
  source 1..137: mol_type = other RNA
cagttgctgcctacattttcaaattccactcaacaaacatgacactcctgggactggaaagaaaaaaaatccaagactgtgtagaataagcaaacaaggatgtcagaagaacccatactgctaaaaaaagccagatc

SEQ ID NO: 52 - RNA, 151 nt, Homo sapiens
  source 1..151: mol_type = unassigned RNA
aaaacaacgttctgtcccccttgccgtcccaagcaatggatgatttgatgctgtccccggacgatattgaacaatagttcactgaagacccaggtccagatgaagctcccagaatgccagaggctgctccccccgtggcccctgcaccagc

SEQ ID NO: 53 - protein, 5 aa, synthetic construct
GGGGS

SEQ ID NO: 54 - RNA, 151 nt, synthetic construct
  source 1..151: mol_type = other RNA
tcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtaca

SEQ ID NO: 55 - RNA, 198 nt, synthetic construct
  source 1..198: mol_type = other RNA
ctgccatcagtcggcgtggactgtagaaccatgccgactgatggcagtcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtaca

SEQ ID NO: 56 - RNA, 305 nt, synthetic construct
  source 1..305: mol_type = other RNA
gccatcagtcgccggtcccaagcccggataaaatgggggcgggaaaccgcctaaccatgccgactgatggcagtcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtacactgccatcagtcggcgtggactgtagaacactgccaatgccggtcccaagcccggataaaagtggagggtacagtccacgc

SEQ ID NO: 57 - RNA, 304 nt, synthetic construct
  source 1..304: mol_type = other RNA
gccatcagtcgccggtcccaagcccggataaaatgggagggggcgggaaaccgcctaaccatgccgactgatggcagtcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtacactgccatcagtcggcgtggactgtagaacactgccaatgccggtcccaagcccggataaaagtgtacagtccacgc

SEQ ID NO: 58 - RNA, 300 nt, synthetic construct
  source 1..300: mol_type = other RNA
gccatcagtcgccggtcccaagcccggataaaatgggggcgggaaaccgcctaaccatgccgactgatggcagtcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtacactgccatcagtcggcgtggactgtagaacactgccaatgccggtcccaagcccggataaaagtgtacagtccacgc

SEQ ID NO: 59 - RNA, 253 nt, synthetic construct
  source 1..253: mol_type = other RNA
aaccatgccgactgatggcagtcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtacactgccatcagtcggcgtggactgtagaacactgccaatgccggtcccaagcccggataaaagtggagggtacagtccacgc

SEQ ID NO: 60 - RNA, 254 nt, synthetic construct
  source 1..254: mol_type = other RNA
gccatcagtcgccggtcccaagcccggataaaatgggagggggcgggaaaccgcctaaccatgccgactgatggcagtcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtacactgccatcagtcggcgtggactgtag

SEQ ID NO: 61 - RNA, 198 nt, synthetic construct
  source 1..198: mol_type = other RNA
aaccatgccgactgatggcagtcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtacactgccatcagtcggcgtggactgtag

SEQ ID NO: 62 - RNA, 151 nt, synthetic construct
  source 1..151: mol_type = other RNA
tcctgtagctaaggccacaaaattatccactgtttttggaacagtctttccgaagagaccaaagatcacccggcccacatcttcatctccaattcgtaggtcaaaatacaccttgacggtgactttgggccccttcttcttctcatcggcc

SEQ ID NO: 63 - RNA, 198 nt, synthetic construct
  source 1..198: mol_type = other RNA
ctgccatcagtcggcgtggactgtagaaccatgccgactgatggcagtcctgtagctaaggccacaaaattatccactgtttttggaacagtctttccgaagagaccaaagatcacccggcccacatcttcatctccaattcgtaggtcaaaatacaccttgacggtgactttgggccccttcttcttctcatcggcc

SEQ ID NO: 64 - RNA, 298 nt, synthetic construct
  source 1..298: mol_type = other RNA
aaaaacaaaaaacaaaaaaaacaaaaaaaaaaccaaaaaaacaaaacacatcctgtagctaaggccacaaaattatccactgtttttggaacagtctttccgaagagaccaaagatcacccggcccacatcttcatctccaattcgtaggtcaaaatacaccttgacggtgactttgggccccttcttcttctcatcggccaaaaacaaaaaacaaaaaaaacaaaaaaaaaaccaaaaaaacaaaacacactgccatcagtcggcgtggactgtagaaccatgccgactgatggcag

SEQ ID NO: 65 - RNA, 151 nt, synthetic construct
  source 1..151: mol_type = other RNA
gccctggatcatgaagtccttgattacacgatggaatttgctgtttttgtagccaaatcctttctctcctgtagccaaggccacaaaattatccactgtttttggaacagtctttccgaagagaccaaagatcacccggcctacatcttca

SEQ ID NO: 66 - RNA, 198 nt, synthetic construct
  source 1..198: mol_type = other RNA
ctgccatcagtcggcgtggactgtagaaccatgccgactgatggcaggccctggatcatgaagtccttgattacacgatggaatttgctgtttttgtagccaaatcctttctctcctgtagccaaggccacaaaattatccactgtttttggaacagtctttccgaagagaccaaagatcacccggcctacatcttca

SEQ ID NO: 67 - RNA, 298 nt, synthetic construct
  source 1..298: mol_type = other RNA
aaaaacaaaaaacaaaaaaaacaaaaaaaaaaccaaaaaaacaaaacacagccctggatcatgaagtccttgattacacgatggaatttgctgtttttgtagccaaatcctttctctcctgtagccaaggccacaaaattatccactgtttttggaacagtctttccgaagagaccaaagatcacccggcctacatcttcaaaaaacaaaaaacaaaaaaaacaaaaaaaaaaccaaaaaaacaaaacacactgccatcagtcggcgtggactgtagaaccatgccgactgatggcag

SEQ ID NO: 68 - RNA, 151 nt, synthetic construct
  source 1..151: mol_type = other RNA
tgtcttggtgctctccaccttccgcaccacctccatgccctctagaactttgccaaacaccacatgcttgccatccagccaggctgtcttgactgtcgtgatgaagaactgggagccgttggtgtctttgcctgcgttggccatgctcacc

SEQ ID NO: 69 - RNA, 198 nt, synthetic construct
  source 1..198: mol_type = other RNA
ctgccatcagtcggcgtggactgtagaaccatgccgactgatggcagtgtcttggtgctctccaccttccgcaccacctccatgccctctagaactttgccaaacaccacatgcttgccatccagccaggctgtcttgactgtcgtgatgaagaactgggagccgttggtgtctttgcctgcgttggccatgctcacc

SEQ ID NO: 70 - RNA, 298 nt, synthetic construct
  source 1..298: mol_type = other RNA
aaaaacaaaaaacaaaaaaaacaaaaaaaaaaccaaaaaaacaaaacacatgtcttggtgctctccaccttccgcaccacctccatgccctctagaactttgccaaacaccacatgcttgccatccagccaggctgtcttgactgtcgtgatgaagaactgggagccgttggtgtctttgcctgcgttggccatgctcaccaaaaacaaaaaacaaaaaaaacaaaaaaaaaaccaaaaaaacaaaacacactgccatcagtcggcgtggactgtagaaccatgccgactgatggcag

SEQ ID NO: 71 - RNA, 151 nt, synthetic construct
  source 1..151: mol_type = other RNA
gatcacatccttcaggggtttatcccggctgtctgtcttggtgctctccaccttccgcaccacctccatgccctccagaactttgccaaacaccacatgcttgccatctagccaggctgtcttgactgtcgtgatgaagaactgggagccg

SEQ ID NO: 72 - RNA, 198 nt, synthetic construct
  source 1..198: mol_type = other RNA
ctgccatcagtcggcgtggactgtagaaccatgccgactgatggcaggatcacatccttcaggggtttatcccggctgtctgtcttggtgctctccaccttccgcaccacctccatgccctccagaactttgccaaacaccacatgcttgccatctagccaggctgtcttgactgtcgtgatgaagaactgggagccg

SEQ ID NO: 73 - RNA, 298 nt, synthetic construct
  source 1..298: mol_type = other RNA
aaaaacaaaaaacaaaaaaaacaaaaaaaaaaccaaaaaaacaaaacacagatcacatccttcaggggtttatcccggctgtctgtcttggtgctctccaccttccgcaccacctccatgccctccagaactttgccaaacaccacatgcttgccatctagccaggctgtcttgactgtcgtgatgaagaactgggagccgaaaaacaaaaaacaaaaaaaacaaaaaaaaaaccaaaaaaacaaaacacactgccatcagtcggcgtggactgtagaaccatgccgactgatggcag

SEQ ID NO: 74 - RNA, 151 nt, synthetic construct
  source 1..151: mol_type = other RNA
agccagcagattctaggtgggacgcaggctcgttggccacagaccacatcacgaccgcggggtggttcttgtccccacgcaccacttcttccatcacctgcatgtggtgatgcagagaaacgttgttgaagaactgcggcagcgccaggcc

SEQ ID NO: 75 - RNA, 198 nt, synthetic construct
  source 1..198: mol_type = other RNA
ctgccatcagtcggcgtggactgtagaaccatgccgactgatggcagagccagcagattctaggtgggacgcaggctcgttggccacagaccacatcacgaccgcggggtggttcttgtccccacgcaccacttcttccatcacctgcatgtggtgatgcagagaaacgttgttgaagaactgcggcagcgccaggcc

SEQ ID NO: 76 - RNA, 298 nt, synthetic construct
  source 1..298: mol_type = other RNA
aaaaacaaaaaacaaaaaaaacaaaaaaaaaaccaaaaaaacaaaacacaagccagcagattctaggtgggacgcaggctcgttggccacagaccacatcacgaccgcggggtggttcttgtccccacgcaccacttcttccatcacctgcatgtggtgatgcagagaaacgttgttgaagaactgcggcagcgccaggccaaaaacaaaaaacaaaaaaaacaaaaaaaaaaccaaaaaaacaaaacacactgccatcagtcggcgtggactgtagaaccatgccgactgatggcag

SEQ ID NO: 77 - RNA, 151 nt, synthetic construct
  source 1..151: mol_type = other RNA
aaaggtcacaggccgggaggggtccaaggatttggtgtgagcgatcaccatcttcaagtagtagccagcagattccaggtgggacgcaggctcgttggccacagaccacatcacgaccgcggggtggttcttgtccctacgcaccacttct

SEQ ID NO: 78 - RNA, 198 nt, synthetic construct
  source 1..198: mol_type = other RNA
ctgccatcagtcggcgtggactgtagaaccatgccgactgatggcagaaaggtcacaggccgggaggggtccaaggatttggtgtgagcgatcaccatcttcaagtagtagccagcagattccaggtgggacgcaggctcgttggccacagaccacatcacgaccgcggggtggttcttgtccctacgcaccacttct

SEQ ID NO: 79 - RNA, 298 nt, synthetic construct
  source 1..298: mol_type = other RNA
aaaaacaaaaaacaaaaaaaacaaaaaaaaaaccaaaaaaacaaaacacaaaaggtcacaggccgggaggggtccaaggatttggtgtgagcgatcaccatcttcaagtagtagccagcagattccaggtgggacgcaggctcgttggccacagaccacatcacgaccgcggggtggttcttgtccctacgcaccacttctaaaaacaaaaaacaaaaaaaacaaaaaaaaaaccaaaaaaacaaaacacactgccatcagtcggcgtggactgtagaaccatgccgactgatggcag

SEQ ID NO: 80 - RNA, 151 nt, synthetic construct
  source 1..151: mol_type = other RNA
atcggcaaaattccaaatgagctctccaaccacgtattttctgcgtttttgatccagacccagatggtactgctccagcagacttttctggtactcttcagtgaacatcagaggtggatcctggtgaaaccctgcaatcgtttctgctcca

SEQ ID NO: 81 - RNA, 198 nt, synthetic construct
  source 1..198: mol_type = other RNA
ctgccatcagtcggcgtggactgtagaaccatgccgactgatggcagatcggcaaaattccaaatgagctctccaaccacgtattttctgcgtttttgatccagacccagatggtactgctccagcagacttttctggtactcttcagtgaacatcagaggtggatcctggtgaaaccctgcaatcgtttctgctcca

SEQ ID NO: 82 - RNA, 298 nt, synthetic construct
  source 1..298: mol_type = other RNA
aaaaacaaaaaacaaaaaaaacaaaaaaaaaaccaaaaaaacaaaacacaatcggcaaaattccaaatgagctctccaaccacgtattttctgcgtttttgatccagacccagatggtactgctccagcagacttttctggtactcttcagtgaacatcagaggtggatcctggtgaaaccctgcaatcgtttctgctccaaaaaacaaaaaacaaaaaaaacaaaaaaaaaaccaaaaaaacaaaacacactgccatcagtcggcgtggactgtagaaccatgccgactgatggcag

SEQ ID NO: 83 - RNA, 151 nt, synthetic construct
  source 1..151: mol_type = other RNA
atcatattcgtccacaaaatgattctgaattagctgtatcgtcaaggcactcttgcctacgccaccagctccaaccaccacaagtttatattcagtcattttcagcaggcctgggagccgctgagcctctggccccgccgccgccttcagt

SEQ ID NO: 84 - RNA, 198 nt, synthetic construct
  source 1..198: mol_type = other RNA
ctgccatcagtcggcgtggactgtagaaccatgccgactgatggcagatcatattcgtccacaaaatgattctgaattagctgtatcgtcaaggcactcttgcctacgccaccagctccaaccaccacaagtttatattcagtcattttcagcaggcctgggagccgctgagcctctggccccgccgccgccttcagt

SEQ ID NO: 85 - RNA, 298 nt, synthetic construct
  source 1..298: mol_type = other RNA
aaaaacaaaaaacaaaaaaaacaaaaaaaaaaccaaaaaaacaaaacacaatcatattcgtccacaaaatgattctgaattagctgtatcgtcaaggcactcttgcctacgccaccagctccaaccaccacaagtttatattcagtcattttcagcaggcctgggagccgctgagcctctggccccgccgccgccttcagtaaaaacaaaaaacaaaaaaaacaaaaaaaaaaccaaaaaaacaaaacacactgccatcagtcggcgtggactgtagaaccatgccgactgatggcag

SEQ ID NO: 86 - RNA, 151 nt, synthetic construct
  source 1..151: mol_type = other RNA
ttgacctgctgtgtcgagaatatccaagagacaggtttctccatcaattactacttgcttcctgtaggaatcctccattgttggatcatattcgtccacaaaatgattctgaattagctgtatcgtcaaggcactcttgcctacgccacca

SEQ ID NO: 87 - RNA, 198 nt, synthetic construct
  source 1..198: mol_type = other RNA
ctgccatcagtcggcgtggactgtagaaccatgccgactgatggcagttgacctgctgtgtcgagaatatccaagagacaggtttctccatcaattactacttgcttcctgtaggaatcctccattgttggatcatattcgtccacaaaatgattctgaattagctgtatcgtcaaggcactcttgcctacgccacca

SEQ ID NO: 88 - RNA, 298 nt, synthetic construct
  source 1..298: mol_type = other RNA
aaaaacaaaaaacaaaaaaaacaaaaaaaaaaccaaaaaaacaaaacacattgacctgctgtgtcgagaatatccaagagacaggtttctccatcaattactacttgcttcctgtaggaatcctccattgttggatcatattcgtccacaaaatgattctgaattagctgtatcgtcaaggcactcttgcctacgccaccaaaaaacaaaaaacaaaaaaaacaaaaaaaaaaccaaaaaaacaaaacacactgccatcagtcggcgtggactgtagaaccatgccgactgatggcag

SEQ ID NO: 89 - RNA, 151 nt, synthetic construct
  source 1..151: mol_type = other RNA
gtccctcattgcactgtactcctcttgacctgctgtgtcgagaatatccaagagacaggtttctccatcaattaccacttgcttcctgtaggaatcctctattgttggatcatattcgtccacaaaatgattctgaattagctgtatcgtc

SEQ ID NO: 90 - RNA, 198 nt, synthetic construct
  source 1..198: mol_type = other RNA
ctgccatcagtcggcgtggactgtagaaccatgccgactgatggcaggtccctcattgcactgtactcctcttgacctgctgtgtcgagaatatccaagagacaggtttctccatcaattaccacttgcttcctgtaggaatcctctattgttggatcatattcgtccacaaaatgattctgaattagctgtatcgtc

SEQ ID NO: 91 - RNA, 298 nt, synthetic construct
  source 1..298: mol_type = other RNA
aaaaacaaaaaacaaaaaaaacaaaaaaaaaaccaaaaaaacaaaacacagtccctcattgcactgtactcctcttgacctgctgtgtcgagaatatccaagagacaggtttctccatcaattaccacttgcttcctgtaggaatcctctattgttggatcatattcgtccacaaaatgattctgaattagctgtatcgtcaaaaacaaaaaacaaaaaaaacaaaaaaaaaaccaaaaaaacaaaacacactgccatcagtcggcgtggactgtagaaccatgccgactgatggcag

SEQ ID NO: 92 - RNA, 151 nt, synthetic construct
  source 1..151: mol_type = other RNA
aaggcaaatcacatttatttcctactaggaccataggtacatcttcagagtccttaactcttttaatttgttctccataatggtgaatatcttcaaatgatttagtattatttatggcaaatacacaaagaaagccctccccagtcctcat

SEQ ID NO: 93 - RNA, 198 nt, synthetic construct
  source 1..198: mol_type = other RNA
ctgccatcagtcggcgtggactgtagaaccatgccgactgatggcagaaggcaaatcacatttatttcctactaggaccataggtacatcttcagagtccttaactcttttaatttgttctccataatggtgaatatcttcaaatgatttagtattatttatggcaaatacacaaagaaagccctccccagtcctcat

SEQ ID NO: 94 - RNA, 298 nt, synthetic construct
  source 1..298: mol_type = other RNA
aaaaacaaaaaacaaaaaaaacaaaaaaaaaaccaaaaaaacaaaacacaaaggcaaatcacatttatttcctactaggaccataggtacatcttcagagtccttaactcttttaatttgttctccataatggtgaatatcttcaaatgatttagtattatttatggcaaatacacaaagaaagccctccccagtcctcataaaaacaaaaaacaaaaaaaacaaaaaaaaaaccaaaaaaacaaaacacactgccatcagtcggcgtggactgtagaaccatgccgactgatggcag

SEQ ID NO: 95 - RNA, 151 nt, synthetic construct
  source 1..151: mol_type = other RNA
tccataacttcttgctaagtcctgagcctgttttgtgtctactgttctagaaggcaaatcacatttatttcctaccaggaccataggtacatcttcagagtccttaactcttttaatttgttctctataatggtgaatatcttcaaatgat

SEQ ID NO: 96 - RNA, 198 nt, synthetic construct
  source 1..198: mol_type = other RNA
ctgccatcagtcggcgtggactgtagaaccatgccgactgatggcagtccataacttcttgctaagtcctgagcctgttttgtgtctactgttctagaaggcaaatcacatttatttcctaccaggaccataggtacatcttcagagtccttaactcttttaatttgttctctataatggtgaatatcttcaaatgat

SEQ ID NO: 97 - RNA, 298 nt, synthetic construct
  source 1..298: mol_type = other RNA
aaaaacaaaaaacaaaaaaaacaaaaaaaaaaccaaaaaaacaaaacacatccataacttcttgctaagtcctgagcctgttttgtgtctactgttctagaaggcaaatcacatttatttcctaccaggaccataggtacatcttcagagtccttaactcttttaatttgttctctataatggtgaatatcttcaaatgataaaaacaaaaaacaaaaaaaacaaaaaaaaaaccaaaaaaacaaaacacactgccatcagtcggcgtggactgtagaaccatgccgactgatggcag

SEQ ID NO: 98 - RNA, 151 nt, synthetic construct
  source 1..151: mol_type = other RNA
atcaaacacctcacaaaacccccggaacttttaaaatacctcttaaagcacttcttgtgttctcttgagggacagcaggtatagtttaccaccttttgaaggaagattaaagtgtgatagttcagggctttactttccattacgcaactga

SEQ ID NO: 99 - RNA, 198 nt, synthetic construct
  source 1..198: mol_type = other RNA
ctgccatcagtcggcgtggactgtagaaccatgccgactgatggcagatcaaacacctcacaaaacccccggaacttttaaaatacctcttaaagcacttcttgtgttctcttgagggacagcaggtatagtttaccaccttttgaaggaagattaaagtgtgatagttcagggctttactttccattacgcaactga

SEQ ID NO: 100 - RNA, 298 nt, synthetic construct
  source 1..298: mol_type = other RNA
aaaaacaaaaaacaaaaaaaacaaaaaaaaaaccaaaaaaacaaaacacaatcaaacacctcacaaaacccccggaacttttaaaatacctcttaaagcacttcttgtgttctcttgagggacagcaggtatagtttaccaccttttgaaggaagattaaagtgtgatagttcagggctttactttccattacgcaactgaaaaaacaaaaaacaaaaaaaacaaaaaaaaaaccaaaaaaacaaaacacactgccatcagtcggcgtggactgtagaaccatgccgactgatggcag

SEQ ID NO: 101 - RNA, 151 nt, synthetic construct
  source 1..151: mol_type = other RNA
ttcttcggttaaaaataggttctagttttacttttctagatttttcttaacagcttatggaacttgaatgcaaaccacacatgcagaaatacttaaattactagcgtgtggaaagatttgagctgcaaactttagacaaaagaaacatgga

SEQ ID NO: 102 - RNA, 198 nt, synthetic construct
  source 1..198: mol_type = other RNA
ctgccatcagtcggcgtggactgtagaaccatgccgactgatggcagttcttcggttaaaaataggttctagttttacttttctagatttttcttaacagcttatggaacttgaatgcaaaccacacatgcagaaatacttaaattactagcgtgtggaaagatttgagctgcaaactttagacaaaagaaacatgga

SEQ ID NO: 103 - RNA, 298 nt, synthetic construct
  source 1..298: mol_type = other RNA
aaaaacaaaaaacaaaaaaaacaaaaaaaaaaccaaaaaaacaaaacacattcttcggttaaaaataggttctagttttacttttctagatttttcttaacagcttatggaacttgaatgcaaaccacacatgcagaaatacttaaattactagcgtgtggaaagatttgagctgcaaactttagacaaaagaaacatggaaaaaacaaaaaacaaaaaaaacaaaaaaaaaaccaaaaaaacaaaacacactgccatcagtcggcgtggactgtagaaccatgccgactgatggcag

SEQ ID NO: 104 - RNA, 151 nt, synthetic construct
  source 1..151: mol_type = other RNA
ggtcagcttcagagtgcggaagcagatatcatagagggcctcgttgtcaatgcaataggtctcatcagtattctccaccaactgatggacggagagggtggcattgtagggctcgaccacggtgtcagacactttgggtgaaggcaccaca

SEQ ID NO: 105 - RNA, 198 nt, synthetic construct
  source 1..198: mol_type = other RNA
ctgccatcagtcggcgtggactgtagaaccatgccgactgatggcagggtcagcttcagagtgcggaagcagatatcatagagggcctcgttgtcaatgcaataggtctcatcagtattctccaccaactgatggacggagagggtggcattgtagggctcgaccacggtgtcagacactttgggtgaaggcaccaca

SEQ ID NO: 106 - RNA, 298 nt, synthetic construct
  source 1..298: mol_type = other RNA
aaaaacaaaaaacaaaaaaaacaaaaaaaaaaccaaaaaaacaaaacacaggtcagcttcagagtgcggaagcagatatcatagagggcctcgttgtcaatgcaataggtctcatcagtattctccaccaactgatggacggagagggtggcattgtagggctcgaccacggtgtcagacactttgggtgaaggcaccacaaaaaacaaaaaacaaaaaaaacaaaaaaaaaaccaaaaaaacaaaacacactgccatcagtcggcgtggactgtagaaccatgccgactgatggcag

SEQ ID NO: 107 - RNA, 151 nt, synthetic construct
  source 1..151: mol_type = other RNA
accaaatacaaagttgtctggtctaaagatctggccaaaaggacctgagcgaacagagtccatggtcccaggttccagatccaccaggatggcacgaggaacatatttgccacctgtggcttcattgtagtacacagagatgcggtccagc

SEQ ID NO: 108 - RNA, 198 nt, synthetic construct
  source 1..198: mol_type = other RNA
ctgccatcagtcggcgtggactgtagaaccatgccgactgatggcagaccaaatacaaagttgtctggtctaaagatctggccaaaaggacctgagcgaacagagtccatggtcccaggttccagatccaccaggatggcacgaggaacatatttgccacctgtggcttcattgtagtacacagagatgcggtccagc

SEQ ID NO: 109 - RNA, 298 nt, synthetic construct
  source 1..298: mol_type = other RNA
aaaaacaaaaaacaaaaaaaacaaaaaaaaaaccaaaaaaacaaaacacaaccaaatacaaagttgtctggtctaaagatctggccaaaaggacctgagcgaacagagtccatggtcccaggttccagatccaccaggatggcacgaggaacatatttgccacctgtggcttcattgtagtacacagagatgcggtccagcaaaaacaaaaaacaaaaaaaacaaaaaaaaaaccaaaaaaacaaaacacactgccatcagtcggcgtggactgtagaaccatgccgactgatggcag

SEQ ID NO: 110 - RNA, 151 nt, synthetic construct
  source 1..151: mol_type = other RNA
aaaagctttttggctcttaagtctttgataaaaggcgtacataattcttgtgtctactgtacagaatactgccgccagctggatttcccaattctgagtaacactctgcaatccaaacagggttcaaccctccaccttacaggcctgcatt

SEQ ID NO: 111 - RNA, 198 nt, synthetic construct
  source 1..198: mol_type = other RNA
ctgccatcagtcggcgtggactgtagaaccatgccgactgatggcagaaaagctttttggctcttaagtctttgataaaaggcgtacataattcttgtgtctactgtacagaatactgccgccagctggatttcccaattctgagtaacactctgcaatccaaacagggttcaaccctccaccttacaggcctgcatt

SEQ ID NO: 112 - RNA, 298 nt, synthetic construct
  source 1..298: mol_type = other RNA
aaaaacaaaaaacaaaaaaaacaaaaaaaaaaccaaaaaaacaaaacacaaaaagctttttggctcttaagtctttgataaaaggcgtacataattcttgtgtctactgtacagaatactgccgccagctggatttcccaattctgagtaacactctgcaatccaaacagggttcaaccctccaccttacaggcctgcattaaaaacaaaaaacaaaaaaaacaaaaaaaaaaccaaaaaaacaaaacacactgccatcagtcggcgtggactgtagaaccatgccgactgatggcag

SEQ ID NO: 113 - RNA, 151 nt, synthetic construct; identical to SEQ ID NO: 14
  source 1..151: mol_type = other RNA

SEQ ID NO: 114 - RNA, 198 nt, synthetic construct
  source 1..198: mol_type = other RNA
ctgccatcagtcggcgtggactgtagaaccatgccgactgatggcagcagttgctgcctacattttcaatattccactcaacaaacattgacacttcctgggactggaaagtaaaaaaaatccaagtactgtgtagaattaagcaaacaagtgatgtttcagaagtaacccattactgcttaaaataaagcctacatc

SEQ ID NO: 115 - RNA, 298 nt, synthetic construct
  source 1..298: mol_type = other RNA
aaaaacaaaaaacaaaaaaaacaaaaaaaaaaccaaaaaaacaaaacacacagttgctgcctacattttcaatattccactcaacaaacattgacacttcctgggactggaaagtaaaaaaaatccaagtactgtgtagaattaagcaaacaagtgatgtttcagaagtaacccattactgcttaaaataaagcctacatcaaaaacaaaaaacaaaaaaaacaaaaaaaaaaccaaaaaaacaaaacacactgccatcagtcggcgtggactgtagaaccatgccgactgatggcag

SEQ ID NO: 116 - RNA, 151 nt, synthetic construct
  source 1..151: mol_type = other RNA
cctaaggcagtggtcacaggctcggtagcgataaagtgcattccagagaaactgtgcagccaccaggggccgttccacgaagatggtctcccccttccggatgagctgtgtggcaaacagcccctttcccttggcgctgctcacgaaacgg

SEQ ID NO: 117 - RNA, 198 nt, synthetic construct
  source 1..198: mol_type = other RNA
ctgccatcagtcggcgtggactgtagaaccatgccgactgatggcagcctaaggcagtggtcacaggctcggtagcgataaagtgcattccagagaaactgtgcagccaccaggggccgttccacgaagatggtctcccccttccggatgagctgtgtggcaaacagcccctttcccttggcgctgctcacgaaacgg

SEQ ID NO: 118 - RNA, 298 nt, synthetic construct
  source 1..298: mol_type = other RNA
aaaaacaaaaaacaaaaaaaacaaaaaaaaaaccaaaaaaacaaaacacacctaaggcagtggtcacaggctcggtagcgataaagtgcattccagagaaactgtgcagccaccaggggccgttccacgaagatggtctcccccttccggatgagctgtgtggcaaacagcccctttcccttggcgctgctcacgaaacggaaaaacaaaaaacaaaaaaaacaaaaaaaaaaccaaaaaaacaaaacacactgccatcagtcggcgtggactgtagaaccatgccgactgatggcag

SEQ ID NO: 119 - RNA, 151 nt, synthetic construct
  source 1..151: mol_type = other RNA
actcatacaggacttgggaggtatccacatcctcttcctcaggattgcctttaccactcagagaaggagctgtggcagtggcaccagaatggattccagagtccaggtaagactgttgctgccagtgactaacagccgcttttctgtctgg

SEQ ID NO: 120 - RNA, 198 nt, synthetic construct
  source 1..198: mol_type = other RNA
ctgccatcagtcggcgtggactgtagaaccatgccgactgatggcagactcatacaggacttgggaggtatccacatcctcttcctcaggattgcctttaccactcagagaaggagctgtggcagtggcaccagaatggattccagagtccaggtaagactgttgctgccagtgactaacagccgcttttctgtctgg

SEQ ID NO: 121 - RNA, 298 nt, synthetic construct
  source 1..298: mol_type = other RNA
aaaaacaaaaaacaaaaaaaacaaaaaaaaaaccaaaaaaacaaaacacaactcatacaggacttgggaggtatccacatcctcttcctcaggattgcctttaccactcagagaaggagctgtggcagtggcaccagaatggattccagagtccaggtaagactgttgctgccagtgactaacagccgcttttctgtctggaaaaacaaaaaacaaaaaaaacaaaaaaaaaaccaaaaaaacaaaacacactgccatcagtcggcgtggactgtagaaccatgccgactgatggcag

SEQ ID NO: 122 - RNA, 171 nt, synthetic construct
  source 1..171: mol_type = other RNA
ccaccatgcccagttgctgcctacactttcaatattccactcaacaaacattgacacttcctgggactggaaagtaaaaaaaatccaagtactgtgtagaattaagcaaacaagtgatgtttcagaagtaacccattactgcttaaaataaagcctacatcaacactctta

SEQ ID NO: 123 - RNA, 218 nt, synthetic construct
  source 1..218: mol_type = other RNA
ctgccatcagtcggcgtggactgtagaaccatgccgactgatggcagccaccatgcccagttgctgcctacactttcaatattccactcaacaaacattgacacttcctgggactggaaagtaaaaaaaatccaagtactgtgtagaattaagcaaacaagtgatgtttcagaagtaacccattactgcttaaaataaagcctacatcaacactctta

SEQ ID NO: 124 - RNA, 151 nt, synthetic construct
  source 1..151: mol_type = other RNA
cctcccatcacgggggccgtagtagaaggccaagagccacagcagggccgtggggggttcggctgccgacatcagcaattgctctgccaccatctcagcccatcctccgaagtgaatgaacaggaaccagctctcaaagggacctccgcag

SEQ ID NO: 125 - RNA, 198 nt, synthetic construct
  source 1..198: mol_type = other RNA
ctgccatcagtcggcgtggactgtagaaccatgccgactgatggcagcctcccatcacgggggccgtagtagaaggccaagagccacagcagggccgtggggggttcggctgccgacatcagcaattgctctgccaccatctcagcccatcctccgaagtgaatgaacaggaaccagctctcaaagggacctccgcag

SEQ ID NO: 126 - RNA, 151 nt, synthetic construct
  source 1..151: mol_type = other RNA
gaggcgcagcatccacaggcggaggcgaaagcagcccggacagctgaggccggaagagggtggggccgcggtggccagggagccggcgccgccacgcgcgggtgggggggactggggttgctcgcgggctccgggcgggcggcgggcgccg

SEQ ID NO: 127 - RNA, 198 nt, synthetic construct
  source 1..198: mol_type = other RNA
ctgccatcagtcggcgtggactgtagaaccatgccgactgatggcaggaggcgcagcatccacaggcggaggcgaaagcagcccggacagctgaggccggaagagggtggggccgcggtggccagggagccggcgccgccacgcgcgggtgggggggactggggttgctcgcgggctccgggcgggcggcgggcgccg

SEQ ID NO: 128 - RNA, 151 nt, synthetic construct; identical to SEQ ID NO: 24
  source 1..151: mol_type = other RNA

SEQ ID NO: 129 - RNA, 331 nt, synthetic construct
  source 1..331: mol_type = other RNA
aaatccgttgaccttaaacggtcgtgtgggttcaagtccctccacccccacgccggaaacgcaatagccgaaaaacaaaaaacaaaaaaaacaaaaaaaaaaccaaaaaaacaaaacacagggtgatgggtgctggccaggacacccactgtatgattgctgtccaacacagccccagcctttgagacctctgcccagagttgttctccatctataagccaagcagagggctgaggctgttggctctctcatacctagcatctggcttcctaccggtaaaaaacaaaaaacaaaacggctattatgcgttaccggcgagacgctacggact

SEQ ID NO: 130 - RNA, 198 nt, synthetic construct; identical to SEQ ID NO: 114
  source 1..198: mol_type = other RNA

SEQ ID NO: 131 - RNA, 51 nt, synthetic construct
  source 1..51: mol_type = other RNA
gcccttgctcactggcagagccctccagcatcgcgagcaggcgctgcctcc

SEQ ID NO: 132 - RNA, 50 nt, synthetic construct
  source 1..50: mol_type = other RNA
gcccttgctcactggcagagccctcagcatcgcgagcaggcgctgcctcc

SEQ ID NO: 133 - RNA, 71 nt, synthetic construct
  source 1..71: mol_type = other RNA
acagctcctcgcccttgctcactggcagagccctccagcatcgcgagcaggcgctgcctcctccgccgctg

SEQ ID NO: 134 - RNA, 70 nt, synthetic construct
  source 1..70: mol_type = other RNA
acagctcctcgcccttgctcactggcagagccctcagcatcgcgagcaggcgctgcctcctccgccgctg

SEQ ID NO: 135 - RNA, 111 nt, synthetic construct
  source 1..111: mol_type = other RNA
gatgggcaccaccccggtgaacagctcctcgcccttgctcactggcagagccctccagcatcgcgagcaggcgctgcctcctccgccgctgcctcctccgccgctgcctcc

SEQ ID NO: 136 - RNA, 110 nt, synthetic construct
  source 1..110: mol_type = other RNA
gatgggcaccaccccggtgaacagctcctcgcccttgctcactggcagagccctcagcatcgcgagcaggcgctgcctcctccgccgctgcctcctccgccgctgcctcc

SEQ ID NO: 137 - RNA, 150 nt, synthetic construct
  source 1..150: mol_type = other RNA
tcgccgtccagctcgaccaggatgggcaccaccccggtgaacagctcctcgcccttgctcactggcagagccctcagcatcgcgagcaggcgctgcctcctccgccgctgcctcctccgccgctgcctcctccgccctgcagcttgtaca

SEQ ID NO: 138 - RNA, 197 nt, synthetic construct
  source 1..197: mol_type = other RNA
ctgccatcagtcggcgtggactgtagaaccatgccgactgatggcagtcgccgtccagctcgaccaggatgggcaccaccccggtgaacagctcctcgcccttgctcactggcagagccctcagcatcgcgagcaggcgctgcctcctccgccgctgcctcctccgccgctgcctcctccgccctgcagcttgtaca

SEQ ID NO: 139 - RNA, 193 nt, synthetic construct
  source 1..193: mol_type = other RNA
ctgccatcagtcggcgtggactgtagaaccatgccgactgatggcagcagttgctgcctacattttcaatattccactcaacaaacattgacacttcctgggactggaaagtaaaaaaaatccaagtactgtgtagaattaagcaaacaaggatgtcagaagaacccatactgcttaaaataaagcctacatc

SEQ ID NO: 140 - RNA, 190 nt, synthetic construct
  source 1..190: mol_type = other RNA
ctgccatcagtcggcgtggactgtagaaccatgccgactgatggcagcagttgctgcctacattttcaatattccactcaacaaacattgacacttcctgggactggaaagaaaaaaaatccaagtactgtgtagaataagcaaacaaggatgtcagaagaacccatactgctaaaataaagcctacatc

SEQ ID NO: 141 - RNA, 284 nt, synthetic construct
  source 1..284: mol_type = other RNA
aaaaacaaaaaacaaaaaaaacaaaaaaaaaaccaaaaaaacaaaacacacagttgctgcctacattttcaaattccactcaacaaacatgacactcctgggactggaaagaaaaaaaatccaagactgtgtagaataagcaaacaaggatgtcagaagaacccatactgctaaaaaaagccacatcaaaaacaaaaaacaaaaaaaacaaaaaaaaaaccaaaaaaacaaaacacactgccatcagtcggcgtggactgtagaaccatgccgactgatggcag

SEQ ID NO: 142 - RNA, 317 nt, synthetic construct
  source 1..317: mol_type = other RNA
aaatccgttgaccttaaacggtcgtgtgggttcaagtccctccacccccacgccggaaacgcaatagccgaaaaacaaaaaacaaaaaaaacaaaaaaaaaaccaaaaaaacaaaacacacagttgctgcctacattttcaaattccactcaacaaacatgacactcctgggactggaaagaaaaaaaatccaagactgtgtagaataagcaaacaaggatgtcagaagaacccatactgctaaaaaaagccacatcaccggtaaaaaacaaaaaacaaaacggctattatgcgttaccggcgagacgctacggact

SEQ ID NO: 143 - RNA, 31 nt, synthetic construct
  source 1..31: mol_type = other RNA
gagaaggagctgtggcagtggcaccagaatg

SEQ ID NO: 144 - RNA, 78 nt, synthetic construct
  source 1..78: mol_type = other RNA
ctgccatcagtcggcgtggactgtagaaccatgccgactgatggcaggagaaggagctgtggcagtggcaccagaatg

SEQ ID NO: 145 - RNA, 51 nt, synthetic construct
  source 1..51: mol_type = other RNA
ttaccactcagagaaggagctgtggcagtggcaccagaatggattccagag

SEQ ID NO: 146 - RNA, 98 nt, synthetic construct
  source 1..98: mol_type = other RNA
ctgccatcagtcggcgtggactgtagaaccatgccgactgatggcagttaccactcagagaaggagctgtggcagtggcaccagaatggattccagag

SEQ ID NO: 147 - RNA, 71 nt, synthetic construct
  source 1..71: mol_type = other RNA
aggattgcctttaccactcagagaaggagctgtggcagtggcaccagaatggattccagagtccaggtaag

SEQ ID NO: 148 - RNA, 118 nt, synthetic construct
  source 1..118: mol_type = other RNA
ctgccatcagtcggcgtggactgtagaaccatgccgactgatggcagaggattgcctttaccactcagagaaggagctgtggcagtggcaccagaatggattccagagtccaggtaag

SEQ ID NO: 149 - RNA, 111 nt, synthetic construct
  source 1..111: mol_type = other RNA
gtatccacatcctcttcctcaggattgcctttaccactcagagaaggagctgtggcagtggcaccagaatggattccagagtccaggtaagactgttgctgccagtgacta

SEQ ID NO: 150 - RNA, 158 nt, synthetic construct
  source 1..158: mol_type = other RNA
ctgccatcagtcggcgtggactgtagaaccatgccgactgatggcaggtatccacatcctcttcctcaggattgcctttaccactcagagaaggagctgtggcagtggcaccagaatggattccagagtccaggtaagactgttgctgccagtgacta

SEQ ID NO: 151 - RNA, 198 nt, synthetic construct
  source 1..198: mol_type = other RNA
ctgccatcagtcggcgtggactgtagaaccatgccgactgatggcaggctggtgcaggggccacggggggagcagcctctggcattctgggagcttcatctggacctgggtcttcagtgaaccattgttcaatatcgtccggggacagcatcaaatcatccattgcttgggacggcaagggggacagaacgttgtttt

SEQ ID NO: 152 - RNA, 198 nt, synthetic construct
  source 1..198: mol_type = other RNA
ctgccatcagtcggcgtggactgtagaaccatgccgactgatggcaggctggtgcaggggccacggggggagcagcctctggcattctgggagcttcatctggacctgggtcttcagtgaaccattgttcaagatcgtccggggacagcatcaaatcatccattgcttgggacggcaagggggacagaacgttgtttt

SEQ ID NO: 153 - RNA, 198 nt, synthetic construct
  source 1..198: mol_type = other RNA
ctgccatcagtcggcgtggactgtagaaccatgccgactgatggcaggctggtgcaggggccacggggggagcagcctctggcagtcggggagcttcatctggacctgggtcttcagtgaaccattgttcaagatcgtccggggacagcatcaaatcatccagtgcttgggacggcaagggggacagaacgttgtttt

SEQ ID NO: 154 - RNA, 197 nt, synthetic construct
  source 1..197: mol_type = other RNA
ctgccatcagtcggcgtggactgtagaaccatgccgactgatggcaggctggtgcaggggccacggggggagcagcctctggcattctgggagcttcatctggacctgggtcttcagtgaaccattgttcaaatcgtccggggacagcatcaaatcatccattgcttgggacggcaagggggacagaacgttgtttt

SEQ ID NO: 155 - RNA, 194 nt, synthetic construct
  source 1..194: mol_type = other RNA
ctgccatcagtcggcgtggactgtagaaccatgccgactgatggcaggctggtgcaggggccacggggggagcagcctctggcatcgggagcttcatctggacctgggtcttcagtgaaccattgttcaaatcgtccggggacagcatcaaatcatccatgcttgggacggcaagggggacagaacgttgtttt

SEQ ID NO: 156 - RNA, 298 nt, synthetic construct
  source 1..298: mol_type = other RNA
aaaaacaaaaaacaaaaaaaacaaaaaaaaaaccaaaaaaacaaaacacagctggtgcaggggccacggggggagcagcctctggcattctgggagcttcatctggacctgggtcttcagtgaaccattgttcaatatcgtccggggacagcatcaaatcatccattgcttgggacggcaagggggacagaacgttgttttaaaaacaaaaaacaaaaaaaacaaaaaaaaaaccaaaaaaacaaaacacactgccatcagtcggcgtggactgtagaaccatgccgactgatggcag

SEQ ID NO: 157 - RNA, 294 nt, synthetic construct
  source 1..294: mol_type = other RNA
aaaaacaaaaaacaaaaaaaacaaaaaaaaaaccaaaaaaacaaaacacagctggtgcaggggccacggggggagcagcctctggcatcgggagcttcatctggacctgggtcttcagtgaaccattgttcaaatcgtccggggacagcatcaaatcatccatgcttgggacggcaagggggacagaacgttgttttaaaaacaaaaaacaaaaaaaacaaaaaaaaaaccaaaaaaacaaaacacactgccatcagtcggcgtggactgtagaaccatgccgactgatggcag

SEQ ID NO: 158 - RNA, 198 nt, synthetic construct
  source 1..198: mol_type = other RNA
ctgccatcagtcggcgtggactgtagaaccatgccgactgatggcaggggtgatgggtgctggccaggacacccactgtatgattgctgtccaacacagccccagcctttgagacctctgcccagagttgttctccatccaacagggccatgagccccatgactgtgagtactggctttcgcagcaactgcacgtggg

SEQ ID NO: 159 - RNA, 198 nt, synthetic construct
  source 1..198: mol_type = other RNA
ctgccatcagtcggcgtggactgtagaaccatgccgactgatggcaggggtgatgggtgctggccaggacacccactgtatgattgctgtccaacacagccccagcctttgagacctctgcccagagttgttctccatctataagccaagcagagggctgaggctgttggctctctcatacctagcatctggcttcct

SEQ ID NO: 160 - RNA, 47 nt, synthetic construct
  source 1..47: mol_type = other RNA
ctgccatcagtcggcgtggactgtagaaccatgccgactgatggcag

SEQ ID NO: 161 - RNA, 147 nt, synthetic construct
  source 1..147: mol_type = other RNA
aaaaacaaaaaacaaaaaaaacaaaaaaaaaaccaaaaaaacaaaacacactgccatcagtcggcgtggactgtagaaccatgccgactgatggcagaaaaacaaaaaacaaaaaaaacaaaaaaaaaaccaaaaaaacaaaacaca

SEQ ID NO: 162 - DNA, 151 bp, synthetic construct
  source 1..151: mol_type = other DNA
gatgtaggctttattttaagcagtaatgggttacttctgaaacatcacttgtttgcttaattctacacagtacttagattttttttactttccagtcccaggaagtgtcaatgtttgttgagtggaatattgaaaatgtaggcagcaactg

(19) United States
(12) Patent Application Publication
(10) Pub. No.: US 2025/0001011 A1
(43) Pub. Date: Jan. 2, 2025

(54) ENGINEERED ADAR-RECRUITING RNAS AND METHODS OF USE THEREOF

(71) Applicants: Peking University, Beijing (CN); Edigene Therapeutics (Beijing) Inc., Beijing (CN)

(72) Inventors: Wensheng WEI, Beijing (CN); Zongyi YI, Beijing (CN); Pengfei YUAN, Beijing (CN)

(21) Appl. No.: 18/684,082

(22) PCT Filed: Aug. 18, 2022
(86) PCT No.: PCT/CN2022/113304

§ 371 Date: Feb. 15, 2024

(30) Foreign Application Priority Data
Aug. 18, 2021 (WO) PCT/CN2021/113290
Apr. 2, 2022 (WO) PCT/CN2022/085144

(51) Int. Cl. A61K 48/00; C12N 9/78; C12N 15/11; C12N 15/86; C12N 15/90

(52) U.S. Cl. CPC A61K 48/0058; C12N 9/78; C12N 15/11; C12N 15/86; C12N 15/90; C12Y 305/04; C12N 2310/20; C12N 2750/14143

(57) ABSTRACT

Provided are methods for editing RNA by introducing a deaminase-recruiting RNA in a host cell for deamination of an adenosine in a target RNA, methods for reducing off-target editing of RNA, deaminase-recruiting RNAs used in the RNA editing methods and compositions and kits comprising the same.

### FIELD

The present application relates to methods and compositions for editing RNAs using engineered linear or circular RNA capable of recruiting an adenosine deaminase to deaminate one or more adenosines in target RNAs.

### BACKGROUND

Genome editing is a powerful tool for biomedical research and development of therapeutics for diseases. Editing technologies using engineered nucleases, such as zinc finger nucleases (ZFNs), transcription activator-like effector nucleases (TALENs), and Cas proteins of CRISPR system have been applied to manipulate the genome in a myriad of organisms. Recently, taking advantage of the deaminase proteins, such as Adenosine Deaminase Acting on RNA (ADAR), new tools were developed for RNA editing. In mammalian cells, there are three types of ADAR proteins, Adar1 (two isoforms, p110 and p150), Adar2 and Adar3 (catalytically inactive). The catalytic substrate of ADAR protein is double-stranded RNA, and ADAR can remove the —NH2 group from an adenosine (A) nucleobase, changing A to inosine (I). (I) is recognized as guanosine (G) and paired with cytidine (C) during subsequent cellular transcription and translation processes. To achieve targeted RNA editing, the ADAR protein or its catalytic domain was fused with a λN peptide, a SNAP-tag or a Cas protein (dCas13b), and a guide RNA was designed to recruit the chimeric ADAR protein to the target site. Alternatively, overexpressing ADAR1 or ADAR2 proteins together with an R/G motif-bearing guide RNA was also reported to enable targeted RNA editing.

However, currently available ADAR-mediated RNA editing technologies have certain limitations. For example, the most effective in vivo delivery for gene therapy is through viral vectors, but the highly desirable adeno-associated virus (AAV) vectors are limited with the cargo size (˜4.5 kb), making it challenging for accommodating both the protein and the guide RNA. Furthermore, over-expression of ADAR1 has recently been reported to confer oncogenicity in multiple myelomas due to aberrant hyper-editing on RNAs, and to generate substantial global off-targeting edits. In addition, ectopic expression of proteins or their domains of non-human origin has potential risk of eliciting immunogenicity. Moreover, pre-existing adaptive immunity and p53-mediated DNA damage response may compromise the efficacy of the therapeutic protein, such as Cas9.

### BRIEF SUMMARY

The present application provides methods of RNA editing using ADAR-recruiting RNAs (“dRNA” or “arRNA”), including circular ADAR-recruiting RNAs (“circ-dRNA” or “circ-arRNA”), which are capable of leveraging endogenous Adenosine Deaminase Acting on RNA (“ADAR”) proteins for the RNA editing. Also provided herein are engineered dRNAs or constructs comprising a nucleic acid sequence encoding the engineered dRNAs used in these methods, and compositions and kits comprising the same. Further provided herein are methods for treating or preventing a disease or condition in an individual comprising editing a target RNA associated with the disease or condition in a cell of the individual.

In one aspect, provided herein is a method for editing a target adenosine in a target RNA in a host cell, comprising introducing a deaminase-recruiting RNA (dRNA) or a construct comprising a nucleic acid sequence encoding the dRNA into the host cell, wherein: (1) the dRNA comprises a targeting RNA sequence that is capable of hybridizing to the target RNA to form a duplex RNA, wherein the duplex RNA comprises a bulge comprising a non-target adenosine in the target RNA; and (2) the dRNA is capable of recruiting an adenosine deaminase acting on RNA (ADAR). In some embodiments, the duplex RNA comprises a bulge at each non-target adenosine in the target RNA. In some embodiments, the targeting RNA sequence is complementary to the target RNA with the exception of one or more nucleotides opposite to non-target adenosines in the target RNA. In some embodiments, the targeting RNA sequence is complementary to the target RNA except for lacking two or more consecutive nucleotides opposite to non-target adenosines in the target RNA. In some embodiments, the method comprises reducing level of editing of non-target adenosine in the target RNA.

In some embodiments according to any one of the methods described above, the dRNA is a linear RNA. In some embodiments the dRNA is a linear RNA capable of forming a circular RNA. In some embodiments, the dRNA is a circular RNA.

In some embodiments according to any one of the methods described above, the dRNA comprises a linker nucleic acid sequence flanking an end of the targeting RNA sequence, wherein the linker nucleic acid sequence does not substantially form any secondary structure with any part of the dRNA.

In some embodiments according to any one of the methods described above, the dRNA comprises a linker nucleic acid sequence replacing an end of the targeting RNA sequence, wherein the linker nucleic acid sequence does not substantially form any secondary structure with any part of the dRNA.

In another aspect, provided herein is a method for editing a target adenosine in a target RNA in a host cell, comprising introducing a dRNA or a construct comprising a nucleic acid sequence encoding the dRNA into the host cell, wherein: (1) the dRNA comprises a targeting RNA sequence that is capable of hybridizing to the target RNA, wherein the dRNA comprises a linker nucleic acid sequence flanking an end of the targeting RNA sequence, wherein the linker nucleic acid sequence does not substantially form any secondary structure with any part of the dRNA; (2) the dRNA is capable of recruiting an ADAR; and (3) the dRNA is a circular RNA or a linear RNA capable of forming a circular RNA. In some embodiments, the dRNA is a circular RNA. In some embodiments, the dRNA is a linear RNA capable of forming a circular RNA.

In some embodiments according to any one of the methods described above, wherein the dRNA comprises a linker nucleic acid sequence, the linker nucleic acid sequence is about 5 nucleotides (nt) to about 500 nt long. In some embodiments, the linker nucleic acid sequence is about 50 nt to about 500 nt long. In some embodiments, the linker nucleic acid sequence is about 5 nucleotides (nt) to about 500 nt long. In some embodiments, the linker nucleic acid sequence is less than or equal to 70 nt in length, optionally wherein the length of the linker nucleic acid sequence is any integer between 10 nt-50 nt, 10 nt-40 nt, 10 nt-30 nt, 10 nt-20 nt, 20 nt-50 nt, 20 nt-40 nt, 20 nt-30 nt, 30 nt-50 nt, 30 nt-40 nt or 40 nt-50 nt. In some embodiments, the linker nucleic acid sequence is about 20 nt to about 60 nt in length; optionally wherein the linker nucleic acid sequence is about 30 nt in length, or about 50 nt in length. In some embodiments, at least about any one of: 50%, 60%, 70%, 80%, 85%, 90%, or 95% of the linker nucleic acid sequence comprises adenosine or cytidine; optionally wherein 100% of the linker nucleic acid sequence comprises adenosine or cytidine. In some embodiments, the linker nucleic acid sequence comprises a polyadenosine (polyA), polyguanosine (polyG), or polycytosine (polyC) sequence. In some embodiments, at least 50% of the linker nucleic acid sequence comprises adenosine. In some embodiments, the linker nucleic acid sequence comprises a dinucleotide repeat sequence. In some embodiments, linker nucleic acid sequence comprises (AT)_{n,} wherein n is an integer greater or equal to 3. In some embodiments, the linker nucleic acid sequence comprises SEQ ID NO: 22.

In some embodiments according to any one of the methods described above, wherein the dRNA comprises a linker nucleic acid sequence, the dRNA comprises a first linker nucleic acid sequence flanking the 5′ end of the targeting RNA sequence and a second linker nucleic acid sequence flanking the 3′ end of the targeting RNA sequence. In some embodiments, the dRNA comprises a first linker nucleic acid sequence flanking the 5′ end of the targeting RNA sequence and a second linker nucleic acid sequence replacing the 3′ end of the targeting RNA sequence. In some embodiments, the dRNA comprises a first linker nucleic acid sequence replacing the 5′ end of the targeting RNA sequence and a second linker nucleic acid sequence flanking the 3′ end of the targeting RNA sequence. In some embodiments, the dRNA comprises a first linker nucleic acid sequence replacing the 5′ end of the targeting RNA sequence and a second linker nucleic acid sequence replacing the 3′ end of the targeting RNA sequence. In some embodiments, the first linker nucleic acid sequence is identical to the second linker nucleic acid sequence. In some embodiments, the first linker nucleic acid sequence is different from the second linker nucleic acid sequence. In some embodiments, the dRNA is a circular RNA, and the linker nucleic acid sequence connects the 5′ end of the targeting RNA sequence and the 3′ end of the targeting RNA sequence.

In some embodiments according to any one of the methods described above, wherein the dRNA is a circular RNA, the dRNA further comprises a 3′ exon sequence recognizable by a 3′ catalytic Group I intron fragment flanking the 5′ end of the targeting RNA sequence, and a 5′ exon sequence recognizable by a 5′ catalytic Group I intron fragment flanking the 3′ end of the targeting RNA sequence.

In some embodiments according to any one of the methods described above, the dRNA further comprises a 3′ ligation sequence and a 5′ ligation sequence. In some embodiments, the 3′ ligation sequence and the 5′ ligation sequence are at least partially complementary to each other. In some embodiments, the 3′ ligation sequence and the 5′ ligation sequence are about 20 to about 75 nucleotides in length. In some embodiments, the ligation sequence is about 5 nucleotides (nt) to about 500 nt long. In some embodiments, the ligation sequence is less than or equal to 70 nt in length, optionally wherein the length of the ligation sequence is any integer between 10 nt-50 nt, 10 nt-40 nt, 10 nt-30 nt, 10 nt-20 nt, 20 nt-50 nt, 20 nt-40 nt, 20 nt-30 nt, 30 nt-50 nt, 30 nt-40 nt or 40 nt-50 nt. In some embodiments, the ligation sequence is about 20 nt to about 60 nt in length; optionally wherein the ligation sequence is about 30 nt in length, or about 50 nt in length. In some embodiments, at least about any one of: 50%, 60%, 70%, 80%, 85%, 90%, or 95% of the ligation sequence comprises adenosine or cytidine; optionally wherein 100% of the ligation sequence comprises adenosine or cytidine. In some embodiments, the dRNA is circularized by RNA ligase RtcB. In some embodiments, the RNA ligase RtcB is expressed endogenously in the host cell. In some embodiments, the dRNA is circularized by T4 RNA ligase 1 (Rnl1) or RNA ligase 2 (Rnl2).

In some embodiments according to any one of the methods described above, the dRNA or the construct comprising a nucleic acid sequence encoding the dRNA edits the target adenosine in the target RNA in a dose dependent manner.

In some embodiments according to any one of the methods described above, the method comprises introducing a construct comprising a nucleic acid sequence encoding the dRNA into the host cell. In some embodiments, the construct further comprises a promoter operably linked to the nucleic acid sequence encoding the dRNA. In some embodiments, the promoter is a polymerase II promoter (“Pol II promoter”). In some embodiments, the promoter is a polymerase III promoter (“Pol III promoter”). In some embodiments, the construct is a viral vector or a plasmid. In some embodiments, the construct is an adeno-associated viral (AAV) vector. In some embodiments, the construct is a self-complementary AAV (scAAV).

In some embodiments according to any one of the methods described above, the ADAR is endogenously expressed by the host cell. In some embodiments, the host cell is a T cell.

In some embodiments according to any one of the methods described above, the targeting RNA sequence is about 100 to about 200 nt (e.g., about 150 nt or about 170 nt) long. In some embodiments, the targeting RNA sequence comprises a cytidine, adenosine or uridine directly opposite the target adenosine in the target RNA. In some embodiments, the targeting RNA sequence comprises a cytidine mismatch directly opposite the target adenosine in the target RNA. In some embodiments, the cytidine mismatch is located at least 20 nucleotides away from the 3′ end of the targeting RNA sequence, and at least 5 nucleotides away from the 5′ end of the targeting RNA sequence. In some embodiments, the 5′ nearest neighbor of the target adenosine in the target RNA is a nucleotide selected from U, C, A and G with the preference U>C≈A>G and the 3′ nearest neighbor of the target adenosine in the target RNA is a nucleotide selected from G, C, A and U with the preference G>C>A≈U. In some embodiments, the target adenosine is in a three-base motif of UAG, and wherein the targeting RNA comprises an A directly opposite the uridine in the three-base motif, a cytidine directly opposite the target adenosine, and a cytidine, guanosine or uridine directly opposite the guanosine in the three-base motif.

In some embodiments according to any one of the methods described above, the target RNA is an RNA selected from the group consisting of a pre-messenger RNA, a messenger RNA, a ribosomal RNA, a transfer RNA, a long non-coding RNA and a small RNA. In some embodiments, the target RNA is a pre-messenger RNA.

In some embodiments according to any one of the methods described above, the method further comprises introducing an inhibitor of ADAR3 and/or a stimulator of interferon into the host cell.

In some embodiments according to any one of the methods described above, the method comprises introducing a plurality of dRNAs or constructs each targeting a different target RNA into the host cell.

In some embodiments according to any one of the methods described above, the efficiency of editing the target RNA is at least 40%.

In some embodiments according to any one of the methods described above, the method further comprises introducing an ADAR into the host cell.

In some embodiments according to any one of the methods described above, deamination of the target adenosine in the target RNA results in a missense mutation, an early stop codon, aberrant splicing, or alternative splicing in the target RNA, or reversal of a missense mutation, an early stop codon, aberrant splicing, or alternative splicing in the target RNA. In some embodiments, deamination of the target adenosine in the target RNA results in point mutation, truncation, elongation and/or misfolding of the protein encoded by the target RNA, or a functional, full-length, correctly-folded and/or wild-type protein by reversal of a missense mutation, an early stop codon, aberrant splicing, or alternative splicing in the target RNA.

In some embodiments according to any one of the methods described above, the host cell is a eukaryotic cell. In some embodiments, the host cell is a mammalian cell. In some embodiments, the host cell is a human or mouse cell.

In another aspect, provided herein is an edited RNA or a host cell having an edited RNA produced by any one of the methods presented above.

In another aspect, provided herein is a method for treating or preventing a disease or condition in an individual, comprising editing a target RNA associated with the disease or condition in a cell of the individual according to the method of any one of the methods described above. In some embodiments, the disease or condition is a hereditary genetic disease or a disease or condition associated with one or more acquired genetic mutations. In some embodiments, the disease or condition is a monogenetic or a polygenetic disease or condition. In some embodiments, the target RNA has a G to A mutation.

In some embodiments according to any one of the methods described above, the target RNA is TP53, and the disease or condition is cancer. In some embodiments, the target RNA is IDUA, and the disease or condition is Mucopolysaccharidosis type I (MPS I). In some embodiments, the target RNA is COL3A1, and the disease or condition is Ehlers-Danlos syndrome. In some embodiments, the target RNA is BMPR2, and the disease or condition is Joubert syndrome. In some embodiments, the target RNA is FANCC, and the disease or condition is Fanconi anemia. In some embodiments, the target RNA is MYBPC3, and the disease or condition is primary familial hypertrophic cardiomyopathy. In some embodiments, the target RNA is IL2RG, and the disease or condition is X-linked severe combined immunodeficiency. In some embodiments, the target RNA is MALAT1, and the disease or condition is Hyperglycemia. In some embodiments, the target RNA is RAB7A, and the disease or condition is Charcot-Marie-Tooth disease 2B (CMT2B).

Also provided are compositions, kits and articles of manufacture for use in any one the methods described above.

In another aspect, provided herein is a dRNA for editing a target RNA comprising a targeting RNA sequence that is that is capable of hybridizing to the target RNA to form a duplex RNA, wherein the duplex RNA comprises a bulge comprising a non-target adenosine in the target RNA. In some embodiments, the duplex RNA comprises a bulge at each non-target adenosine in the target RNA. In some embodiments, the targeting RNA sequence is complementary to the target RNA with the exception of one or more nucleotides opposite to non-target adenosines in the target RNA. In some embodiments, the targeting RNA sequence is complementary to the target RNA with the exception of two or more consecutive nucleotides opposite to non-target adenosines in the target RNA. In some embodiments, the method comprises reducing level of editing of non-target adenosine in the target RNA.

In some embodiments according to any one of the dRNAs described above, the dRNA is a linear RNA. In some embodiments the dRNA is a linear RNA capable of forming a circular RNA. In some embodiments, the dRNA is a circular RNA.

In some embodiments according to any one of the dRNAs described above, the dRNA comprises a linker nucleic acid sequence flanking an end of the targeting RNA sequence, wherein the linker nucleic acid sequence does not substantially form any secondary structure with any part of the dRNA. In some embodiments, the dRNA comprises a linker nucleic acid sequence replacing an end of the targeting RNA sequence, wherein the linker nucleic acid sequence does not substantially form any secondary structure with any part of the dRNA.

In another aspect presented herein is a dRNA for editing a target RNA comprising a targeting RNA sequence that is capable of hybridizing to the target RNA, wherein the dRNA comprises a linker nucleic acid sequence flanking an end of the targeting RNA sequence, wherein the linker nucleic acid sequence does not substantially form any secondary structure with any part of the dRNA, and wherein the dRNA is a circular RNA or a linear RNA capable of forming a circular RNA. In some embodiments, the dRNA is a circular RNA. In some embodiments, the dRNA is a linear RNA capable of forming a circular RNA.

In an additional aspect presented herein is a dRNA for editing a target RNA comprising a targeting RNA sequence that is capable of hybridizing to the target RNA, wherein the dRNA comprises a linker nucleic acid sequence replacing an end of the targeting RNA sequence, wherein the linker nucleic acid sequence does not substantially form any secondary structure with any part of the dRNA, and wherein the dRNA is a circular RNA or a linear RNA capable of forming a circular RNA. In some embodiments, the dRNA is a circular RNA. In some embodiments, the dRNA is a linear RNA capable of forming a circular RNA.

In some embodiments according to any one of the dRNAs described above, wherein the dRNA comprises a linker nucleic acid sequence, the linker nucleic acid sequence is about 5 nucleotides (nt) to about 500 nt long. In some embodiments, the linker nucleic acid sequence is about 50 nt to about 500 nt long. In some embodiments, the linker nucleic acid sequence is about 5 nucleotides (nt) to about 500 nt long. In some embodiments, the linker nucleic acid sequence is less than or equal to 70 nt in length, optionally wherein the length of the linker nucleic acid sequence is any integer between 10 nt-50 nt, 10 nt-40 nt, 10 nt-30 nt, 10 nt-20 nt, 20 nt-50 nt, 20 nt-40 nt, 20 nt-30 nt, 30 nt-50 nt, 30 nt-40 nt or 40 nt-50 nt. In some embodiments, the linker nucleic acid sequence is about 20 nt to about 60 nt in length; optionally wherein the linker nucleic acid sequence is about 30 nt in length, or about 50 nt in length. In some embodiments, at least about any one of: 50%, 60%, 70%, 80%, 85%, 90%, or 95% of the linker nucleic acid sequence comprises adenosine or cytidine; optionally wherein 100% of the linker nucleic acid sequence comprises adenosine or cytidine. In some embodiments, the linker nucleic acid sequence comprises a polyadenosine (polyA), polyguanosine (polyG), or polycytosine (polyC) sequence. In some embodiments, at least 50% of the linker nucleic acid sequence comprises adenosine. In some embodiments, the linker nucleic acid sequence comprises a dinucleotide repeat sequence. In some embodiments, linker nucleic acid sequence comprises (AT)_{n,} wherein n is an integer greater or equal to 3.

In some embodiments according to any one of the dRNAs described above, wherein the dRNA comprises a linker nucleic acid sequence, the dRNA comprises a first linker nucleic acid sequence flanking the 5′ end of the targeting RNA sequence and a second linker nucleic acid sequence flanking the 3′ end of the targeting RNA sequence. In some embodiments, the dRNA comprises a first linker nucleic acid sequence flanking the 5′ end of the targeting RNA sequence and a second linker nucleic acid sequence replacing the 3′ end of the targeting RNA sequence. In some embodiments, the dRNA comprises a first linker nucleic acid sequence replacing the 5′ end of the targeting RNA sequence and a second linker nucleic acid sequence flanking the 3′ end of the targeting RNA sequence. In some embodiments, the dRNA comprises a first linker nucleic acid sequence replacing the 5′ end of the targeting RNA sequence and a second linker nucleic acid sequence replacing the 3′ end of the targeting RNA sequence. In some embodiments, the first linker nucleic acid sequence is identical to the second linker nucleic acid sequence. In some embodiments, the first linker nucleic acid sequence is different from the second linker nucleic acid sequence. In some embodiments, the dRNA is a circular RNA, and the linker nucleic acid sequence connects the 5′ end of the targeting RNA sequence and the 3′ end of the targeting RNA sequence.

In some embodiments according to any one of the dRNAs described above, wherein the dRNA is a circular RNA, the dRNA further comprises a 3′ exon sequence recognizable by a 3′ catalytic Group I intron fragment flanking the 5′ end of the targeting RNA sequence, and a 5′ exon sequence recognizable by a 5′ catalytic Group I intron fragment flanking the 3′ end of the targeting RNA sequence.

In some embodiments according to any one of the dRNAs described above, the dRNA further comprises a 3′ ligation sequence and a 5′ ligation sequence. In some embodiments, the 3′ ligation sequence and the 5′ ligation sequence are at least partially complementary to each other. In some embodiments, the 3′ ligation sequence and the 5′ ligation sequence are about 20 to about 75 nucleotides in length. In some embodiments, the dRNA is circularized by RNA ligase RtcB. In some embodiments, the RNA ligase RtcB is expressed endogenously in the host cell. In some embodiments, the dRNA is circularized by T4 RNA ligase 1 (Rnl1) or RNA ligase 2 (Rnl2).

In some embodiments according to any one of the dRNAs described above, the targeting RNA sequence is about 100 to about 200 nt (e.g., about 150 nt or about 170 nt) long. In some embodiments, the targeting RNA sequence comprises a cytidine, adenosine or uridine directly opposite the target adenosine in the target RNA. In some embodiments, the targeting RNA sequence comprises a cytidine mismatch directly opposite the target adenosine in the target RNA. In some embodiments, the cytidine mismatch is located at least 20 nucleotides away from the 3′ end of the targeting RNA sequence, and at least 5 nucleotides away from the 5′ end of the targeting RNA sequence. In some embodiments, the 5′ nearest neighbor of the target adenosine in the target RNA is a nucleotide selected from U, C, A and G with the preference U>C≈A>G and the 3′ nearest neighbor of the target adenosine in the target RNA is a nucleotide selected from G, C, A and U with the preference G>C>A≈U. In some embodiments, the target adenosine is in a three-base motif of UAG, and wherein the targeting RNA comprises an A directly opposite the uridine in the three-base motif, a cytidine directly opposite the target adenosine, and a cytidine, guanosine or uridine directly opposite the guanosine in the three-base motif.

In some embodiments according to any one of the dRNAs described above, the target RNA is an RNA selected from the group consisting of a pre-messenger RNA, a messenger RNA, a ribosomal RNA, a transfer RNA, a long non-coding RNA and a small RNA. In some embodiments, the target RNA is a pre-messenger RNA.

In some embodiments, there is provided a construct comprising a nucleic acid sequence encoding any one of the dRNAs described above. In some embodiments, the construct further comprises a promoter operably linked to the nucleic acid sequence encoding the dRNA, wherein the promoter is a Pol III promoter. In some embodiments, the construct is a viral vector or a plasmid. In some embodiments, the construct is an adeno-associated viral (AAV) vector. In some embodiments, the construct is a self-complementary AAV (scAAV).

In some embodiments, there is provided a host cell comprising any one of the constructs or dRNAs described above. In some embodiments, there is provided a kit comprising any one of the constructs or dRNAs described above, wherein the kit further comprises instructions for editing a target RNA in a host cell.

It is to be understood that one, some, or all of the properties of the various embodiments described herein may be combined to form other embodiments of the present application. These and other embodiments of the present application are further described by the detailed description that follows.

### BRIEF DESCRIPTION OF THE DRAWINGS

FIGS. 1A-1C depict FACS analysis after transfection of a plasmid expressing arRNA driven by a pol II promoter (CMV) and a plasmid expressing arRNA driven by a Pol III promoter (U6). FIG. 1A shows a schematic of genetically encoding arRNA by U6 or CMV promoter. The 151-nt arRNA targeting fluorescence Reporter-1 was expressed under human U6 or CMV promoter. For Reporter 1, mCherry and EGFP genes were linked by sequences containing 3×GGGGS-coding region and an in-frame UAG stop codon. The reporter-expressed cells only produced mCherry protein, while targeted editing on the UAG stop codon of the reporter transcript could convert the UAG to UIG and thus to permit the downstream EGFP expression. FIG. 1B shows FACS results of expression level of arRNA, normalized with GAPDH. FIG. 1C shows FACS results of EGFP⁺ percentage showing the editing efficiency of different promoter driven-arRNA targeting the reporter transcripts in reporter-stably-expressing HEK293T cells. The ratios of EGFP+ cells were normalized by transfection efficiency, which was determined by mCherry⁺.

FIGS. 2A-2N depict editing efficacy of circular ADAR recruiting RNAs (circ-arRNA) and show that Circ-arRNAs enable efficient and long-lasting programmable RNA editing on endogenous transcripts. FIG. 2A shows a schematic of genetically encoding circ-arRNA targeting fluorescence Reporter-1. FIG. 2B depicts quantitative PCR results showing the expressions level of linear arRNAs and circ-arRNAs. Data is presented as the mean±S.E.M. (n=3), n represents the number of independent experiments performed in parallel; unpaired two-sided Student's t-test, ****P<0.0001. FIG. 2C shows FACS results of EGFP⁺ percentage showing the editing efficiency of circ-arRNA driven by U6 and CMV promoter targeting the reporter transcripts in reporter-stably-expressing HEK293T cells. EGFP⁺ percentages were normalized by transfection efficiency, which was determined by mCherry⁺. Data is presented as the mean±S.E.M. (n=3), n represents the number of independent experiments performed in parallel; unpaired two-sided Student's t-test, ****P<0.0001. FIG. 2D shows the FACS results of EGFP⁺ percentage showing the editing efficiency of different arRNA versions targeting the reporter transcripts in reporter-stably-expressing HEK293T cells. EGFP⁺ percentages were normalized by transfection efficiency, which was determined by mCherry⁺. FIG. 2E shows EGFP expression in reporter-stable-expressing HEK293T cells after transfected by U6-driven linear arRNA and circ-arRNA at day 2, day 9 and day 18. Scale bar=200 μm. Data are mean values±S.E.M. (n=3), n represents the number of independent experiments performed in parallel. FIG. 2F shows FACS results of EGFP⁺ percentage showing dependency of arRNA and circ-arRNA on the endogenous ADAR. EGFP⁺ percentages were normalized by transfection efficiency, which was determined by mCherry⁺. Data are mean values±S.E.M. (n=3), n represents the number of independent experiments performed in parallel. FIG. 2G shows FACS results of EGFP⁺ percentage showing the editing efficiency of different version of arRNA targeting reporter transcripts in multiple cell lines. EGFP⁺ percentages were normalized by transfection efficiency, which was determined by mCherry⁺. FIG. 2H shows a schematic of endogenous transcripts of six genes (PPIA, KRAS, RAB7A, FANCC, MALAT1, and TP53) and their corresponding arRNA/circ-arRNA. FIG. 2I shows deep sequencing results showing the editing rate on targeted adenosine of the PPIA, KRAS, RAB7A, FANCC, MALAT1, and TP53 transcripts by U6-driven linear arRNA and circ-arRNA in HEK293T cells. Data are mean values±S.E.M. (n=3), n represents the number of independent experiments performed in parallel. FIG. 2J shows respective fold change of editing rate normalized to linear arRNAs. Significance was analyzed using a two-tailed unpaired Student's t-test; ****P=1.33124×10⁻¹⁰ for circ-arRNA₁₅₁ and P=1.20289×10⁻¹⁰ for circ-arRNA_{151_}AC50; center line, medians; limits, 75% and 25%; whiskers, maximum and minimum; n=3 per site, 20 sites. FIG. 2K demonstrates NGS results showing rates of targeted editing in HEK293T cells infected by AAV-delivered circ-arRNAs, n=2, mean±s.d. FIG. 2L demonstrates NGS results showing rates of targeted editing in human primary hepatocytes infected by AAV-delivered circ-arRNAs, n=2, mean±s.d. FIG. 2M demonstrates NGS results showing rates of targeted editing in cerebral organoid PPIA infected by AAV-delivered circ-arRNAs, n=2, mean±s.d. FIG. 2N demonstrates NGS results showing rates of targeted editing in cerebral organoid FANCC infected by AAV-delivered circ-arRNAs, n=4, mean±s.d.

FIGS. 3A-3E depict endogenous ADAR protein for targeted RNA editing by in vitro cyclized circular ADAR recruiting RNAs (circ-arRNA). FIG. 3A shows HPLC chromatogram results of circ-arRNA cyclized by in vitro transcription. Top, precursor without T4 Rnl treated. Middle, circ-arRNA ligated by T4 Rnl1. Bottom, circ-arRNA ligated by T4 Rnl2. FIG. 3B shows deep sequencing analysis of A-to-I conversion rates at the targeted site in reporter transcripts. Data indicates mean values±S.E.M. FIG. 3C shows deep sequencing results showing the editing rate on targeted adenosine of the PPIB transcripts by introducing T4 Rnl cyclized circ-arRNA into HEK293T cells. FIG. 3D shows deep sequencing results showing the editing rate on targeted adenosine of the Idua transcripts by introduction of group I ribozyme autocatalysis ligated circ-arRNA into primary MEF cell line generated from Hurler syndrome mice. Data indicates mean values±S.E.M. n=2 or 3, n represents the number of independent experiments performed in parallel. FIG. 3E displays electropherograms showing Sanger sequencing results of the targeted region after transfection with precursor (top), T4 RNA ligase 1-ligated circ-arRNAs (middle) and T4 RNA ligase 2-ligated circ-arRNAs (bottom).

FIGS. 4A-4J depict transcriptome-wide specificity of RNA editing by circ-arRNA, off-target sites analysis of circ-arRNA₁₅₁-PPIA, and show that expression level and splicing pattern of PPIA were not affected by circ-arRNA₁₅₁-PPIA. FIGS. 4A-4B show results of transcriptome-wide off-targeting analysis of circ-arRNA₁₅₁ and ADAR2_{DD} overexpression groups. The on-targeting site for PPIA is shown in FIG. 4A. Potential off-target sites identified in PPIA targeting RNA groups and ADAR2_{DD} overexpression groups are unmarked. FIG. 4C shows transcriptome-wide analysis of the effects of circ-arRNA₁₅₁-PPIA on native editing sites. Three independent experiments were performed. FIG. 4D shows the distribution of off-target sites. FIG. 4E shows minimal free energy of circ-arRNA₁₅₁ and off-target sites area by RNAhybrid. FIG. 4F shows differential gene expression analysis of the effects of Ctrl RNA₁₅₁ and circ-arRNA₁₅₁ from RNA-seq data at the transcriptome level. FPKM value was calculated with the STRINGTIE tool, and Pearson's correlation coefficient analysis was used to assess global differential gene expression. FIG. 4G displays immunoblot showing PPIA protein expression level in cells transfected by Ctrl RNA₁₅₁ and circ-arRNA₁₅₁-PPIA. FIG. 4H demonstrates quantitative PCR showing the effects of circarRNAs vs. control RNA on the expression levels of targeted PPIA transcripts in HEK293T cells, normalized to GAPDH; n=3, mean±SD. FIG. 4I shows the FPKM of two main PPIA splicing isoforms, ns, not significance; n=2, mean±SD. Unpaired twotailed Student's t-test, ns, not significant. FIG. 4J shows the splicing junction of targeted gene. Top, PPIA splicing isoforms in RNA-seq analysis. Middle, the PPIA splicing junction of Ctrl RNA₁₅₁. Bottom, the PPIA splicing junction of circ-arRNA₁₅₁.

FIGS. 5A-5M depict bystander off-target editing by engineered circ-arRNA, and relative quantitative expression level of ADAR and interferon-stimulated genes. FIG. 5A shows a schematic of arRNA, with a deletion of the nucleotide opposite to the targeted adenosine (linear arRNA_{ΔC}), targeting fluorescence Reporter-1 (top) and EGFP⁺ percentage showing the editing efficiency targeting the reporter transcripts of arRNA and arRNA_{ΔC} in reporter-stably-expressing HEK293T cells (bottom). FIG. 5B shows a schematic of circ-arRNA, with a deletion of the nucleotide opposite to the targeted adenosine (circ-arRNA_{ΔC}), targeting fluorescence Reporter-1 (top) and EGFP⁺ percentage showing the editing efficiency targeting the reporter transcripts of circ-arRNA and circ-arRNA_{ΔC} in reporter-stably-expressing HEK293T cells (bottom). FIG. 5C shows a schematic of the PPIA transcript sequence targeted by circ-arRNA₁₅₁ (top) and circ-arRNA_{151-AΔ8} (bottom). The targeted adenosine is at position 76. circ-arRNA_{151-AΔ8} has an adenosine deletion on A^{18th}, A^{25th}, A^{33rd}, A^{41st}, A^{42nd}, A^{47th}, A^{59th}, and A^{87th} to minimize the potential off-targets on editing-prone motifs. FIG. 5D shows the on-target editing rate of PPIA transcripts of circ-arRNA₁₅₁ and circ-arRNA_{151-AΔ8}. FIG. 5E shows the off-target editing rate on A^{18th}, A^{25th}, A^{33rd}, A^{41st}, A^{42nd}, A^{47th}, A^{59th}, and A^{87th} of circ-arRNA₁₅₁ and circ-arRNA_{151-AΔ8}. FIG. 5F shows schematic of PPIA transcript sequence covered by 151-nt arRNA. Black arrow indicates the targeted adenosine, and potential off-target adenosines are marked in red. Circ-arRNA_{151-AΔ14} targeting PPIA transcripts with U deletion opposite A6th, A13th, A18th, A25th, A33rd, A41st, A42nd, A47th, A59th, A72nd, A87th, A103rd, A110th and A129th to minimize potential off-target edits in editing-prone motifs. FIG. 5G shows editing rates of off-target adenosines in circ-arRNA_{151_}AC50 and circ-arRNA_{151-AΔ14_}AC50 groups; n=3, mean±s.d. FIG. 5H shows a heatmap of editing rate for adenosines covered by circ-arRNA_{151_}AC50 and circ-arRNA_{151-AΔ14_}AC50 in PPIA transcripts; n=3. Gray triangles represent positions of U deletions in circ-arRNA_{151-AΔ5}, blue triangles represent positions of additional U deletions in circ-arRNA_{151-AΔ8} based on circ-arRNA_{151-AΔ5}. Positions of U deletions in circ-arRNA_{151-AΔ14_}AC50 denoted by gray, blue and black triangles. FIG. 5I displays IGV results showing editing reads in circRNA_{151_}AC50. FIG. 5J displays IGV results showing editing reads in circ-arRNA_{151-AΔ14_}AC50. FIG. 5K shows that Circ-arRNA₁₅₁₋AΔ14 cyclized in vitro achieves efficient RNA editing in targeted site in dose-dependent way. n=2, mean±SD. FIG. 5L displays the heatmap shown relative expression level of ADAR1p110, ADAR1p150 and ADAR2, normalized to GAPDH. FIG. 5M displays the heatmap showing relative expression level of interferon-stimulated genes, normalized to GAPDH; n=2, mean±SD.

FIGS. 6A-6G depict restoration of transcriptional regulatory activity of mutant TP53^{W53X} by circ-arRNA. FIG. 6A shows a schematic of the TP53 transcript sequence targeted by circ-arRNA₁₅₁_AC50 (top) and circ-arRNA_{151-AΔ4}_AC50 (bottom). FIG. 6B shows deep sequencing results showing the targeted editing on TP53^{W53X} transcripts by circ-arRNA₁₅₁, circ-arRNA_{151-AG1}, circ-arRNA_{151-AG4}, circ-arRNA_{151-AΔ1}, circ-arRNA_{151-AΔ4}, circ-arRNA₁₅₁_AC50, and circ-arRNA_{151-AΔ4}_AC50. FIG. 6C shows a Western blot showing the recovered production of full-length p53 protein from the TP53^{W53X} transcripts in the HEK293T TP53^{-/-} cells by circ-arRNA. FIG. 6D shows detection of the transcriptional regulatory activity of restored p53 protein using a p53-Firefly luciferase reporter system, normalized by co-transfected Renilla-luciferase vector. FIG. 6E shows deep sequencing results of off-target editing rate on A^{18th}, A^{25th}, A^{33rd}, A^{41st}, A^{42nd}, A^{47th}, A^{59th}, and A^{87th} of circ-arRNA₁₅₁ and circ-arRNA_{151-AΔ8}. All data indicates mean values±S.E.M. (n=3), n represents the number of independent experiments performed in parallel. FIG. 6F shows schematic of the TP53^{W53X} transcript sequence covered by the 151-nt arRNA containing a c.158G-to-A clinically relevant non-sense mutation (Trp53Ter). Black arrow indicates the targeted adenosine. The design of circ-arRNAs targeting TP53^{W53X} transcripts with a U deletion opposite A^{66th}, together with A^{36th}, A^{111th}, and A^{114th}, to minimize potential off-target edits in editing-prone motifs. FIG. 6G shows a heatmap of the editing rates on adenosines covered by the indicated circ-arRNAs in the TP53^{W53X} transcript (highlighted in blue). Δ indicates a U deletion site of circ-arRNA; n=3.

FIGS. 7A-7C depict restoration of IDUA activity in Hurler syndrome mice by circ-arRNA. FIG. 7A shows deep sequencing results showing the editing rates on targeted adenosine of Idua transcripts in mice liver cells. FIG. 7B shows the catalytic activity of IDUA with 4-methylumbelliferyl IDUA substrate. FIG. 7C shows quantitative PCR results showing expressions level of Idua transcripts in different treatments. Signal is normalized with GAPDH transcripts. All data is presented as the mean±S.E.M. (n≥3), n represents the number of independent experiments performed in parallel; unpaired two-sided Student's t-test, *P<0.05; **P<0.01.

FIG. 8A shows a schematic of a circular arRNA (circ-arRNA) having flanking linker sequences at the 5′ and 3′ end of the arRNA sequence.

FIG. 8B shows locations of the flanking linker sequences in various designs of circular arRNAs.

FIG. 8C shows on-target editing efficiency of various circ-arRNAs targeting mf-PPIA-UTR2 in LLC-MK2 and FRHK-4 cells.

FIG. 8D shows editing (i.e., A to G conversion) rates at each adenosine (with positions labeled in the x-axis) in the mf-PPIA-UTR2 amplicon using circ-arRNA₁₇₁, circ-arRNA₁₇₁-L, circ-arRNA₁₇₁-R and circ-arRNA₁₇₁-LR in LLC-MK2 cells.

FIG. 8E shows on-target editing efficiency of various circ-arRNAs targeting mf-PPIA-3 in LLC-MK2 and FRHK-4 cells.

FIG. 8F shows editing rates at each adenosine in the mf-PPIA-3 amplicon using circ-arRNA₁₇₁, circ-arRNA₁₇₁-R, and unrelated arRNA (UT) in FRHK-4 cells.

FIG. 8G shows on-target editing efficiency of various circ-arRNAs targeting mf-IDUA-1 in LLC-MK2 and FRHK-4 cells.

FIG. 8H shows editing rates at each adenosine in the mf-IDUA-1 amplicon using circ-arRNA₁₇₁, circ-arRNA₁₇₁+LR, and unrelated arRNA (UT) in LLC-MK2 cells.

FIG. 9A shows a heatmap of editing rates at positions 127 (off 4), 132 (off 3), 155 (on target), 160 (off 2) and 168 (off 1) of the mf-PPIA-3 amplicon using circ-arRNA₁₇₁, and circ-arRNA constructs with U deletions (−1, −2, −3, −4, −43, −432, and −4321) in FRHK and LLC-MK2 cells.

FIG. 9B shows a heatmap of editing rates at positions 62 (off 4), 91 (off 3), 102 (off 2), 118 (off 1), and 134 (on target) of the mf-IDUA-1 amplicon using circ-arRNA₁₇₁, and circ-arRNA constructs with U deletions (−1, −2, −3, −4, −43, −432, and −4321) in FRHK and LLC-MK2 cells.

### DETAILED DESCRIPTION

The present application provides improved RNA editing methods and specially designed RNAs, referred herein as deaminase-recruiting RNAs (“dRNAs”) or ADAR-recruiting RNAs (“arRNAs”) or constructs comprising nucleic acids encoding these arRNAs, to edit target RNAs in a host cell.

“LEAPER” (Leveraging Endogenous ADAR for Programmable Editing on RNA) have been previously developed by inventors of the present application, which leverages endogenous ADAR to edit target RNA by utilizing dRNAs. LEAPER method was described in WO2021/008447 and PCT/CN2021/071292, which are incorporated herein by reference in their entirety. Specifically, a targeting RNA that is partially complementary to the target transcript was used to recruit native ADAR1 or ADAR2 to change adenosine to inosine at a specific site in a target RNA. As such, RNA editing can be achieved in certain systems without ectopic or overexpression of the ADAR proteins in the host cell.

The present application provides improved LEAPER methods that allow for increased editing efficiency, decreased off-target (also referred herein as “bystander editing”) effects, and/or more precise and long-lasting RNA editing. In some embodiments, the dRNA is linear or circular, wherein the dRNA contains deletion of one or more uridines that would base-pair with non-target adenosines in the target RNA. In some embodiments, the dRNA is a circular RNA or a linear RNA capable of forming a circular RNA, wherein the circular RNA comprises one or more linker sequences flanking the targeting RNA sequence that is partially complementary to the target RNA. The linker sequence(s) may enhance on-target editing efficiency for a circular dRNA having a targeting RNA sequence that is prone to form secondary structure(s). The circular dRNAs described herein may provide enhanced stability and efficacy compared to linear dRNAs. The methods described herein have been successfully used to correct pathogenic point mutations. The improved LEAPER methods may provide broad applicability for both therapeutics and biomedical research.

Accordingly, one aspect of the present application provides a method for editing a target adenosine in a target RNA in a host cell, comprising introducing a deaminase-recruiting RNA (dRNA) or a construct comprising a nucleic acid sequence encoding the dRNA into the host cell, wherein: (1) the dRNA comprises a targeting RNA sequence that is capable of hybridizing to the target RNA to form a duplex RNA, wherein the duplex RNA comprises a bulge comprising a non-target adenosine in the target RNA; and (2) the dRNA is capable of recruiting an adenosine deaminase acting on RNA (ADAR).

Another aspect of the present application provides a method for editing a target adenosine in a target RNA in a host cell, comprising introducing a dRNA or a construct comprising a nucleic acid sequence encoding the dRNA into the host cell, wherein: (1) the dRNA comprises a targeting RNA sequence that is capable of hybridizing to the target RNA, wherein the dRNA comprises a linker nucleic acid sequence flanking an end of the targeting RNA sequence, wherein the linker nucleic acid sequence does not substantially form any secondary structure with any part of the dRNA; (2) the dRNA is capable of recruiting an ADAR; and (3) the dRNA is a circular RNA or a linear RNA capable of forming a circular RNA.

A further aspect of the present application provides a method for editing a target adenosine in a target RNA in a host cell, comprising introducing a dRNA or a construct comprising a nucleic acid sequence encoding the dRNA into the host cell, wherein: (1) the dRNA comprises a targeting RNA sequence that is capable of hybridizing to the target RNA, wherein the dRNA comprises a linker nucleic acid sequence replacing an end of the targeting RNA sequence, wherein the linker nucleic acid sequence does not substantially form any secondary structure with any part of the dRNA; (2) the dRNA is capable of recruiting an ADAR; and (3) the dRNA is a circular RNA or a linear RNA capable of forming a circular RNA.

### I. Definitions

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions are specifically embraced by the present disclosure and are disclosed herein just as if each and every combination was individually and explicitly disclosed.

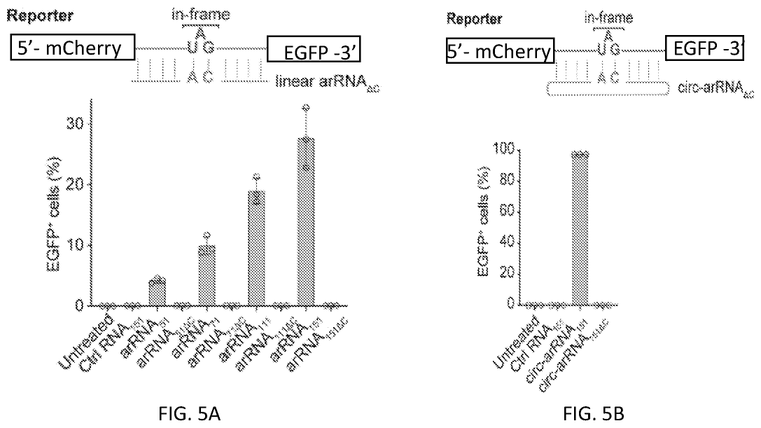

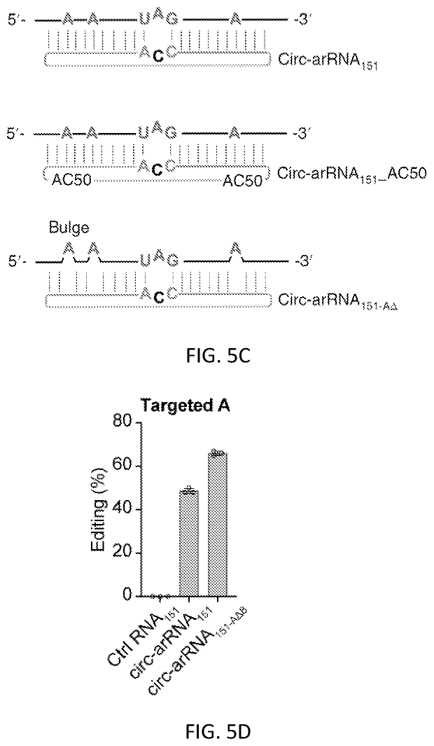

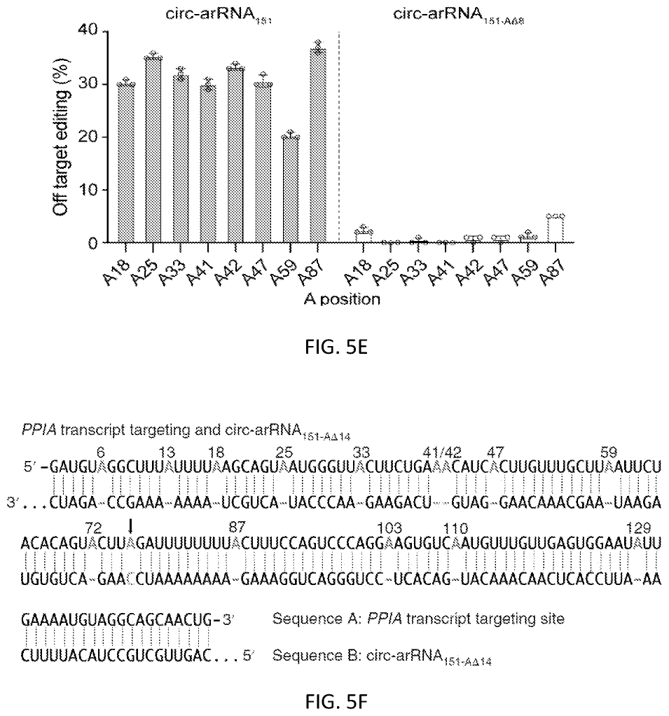

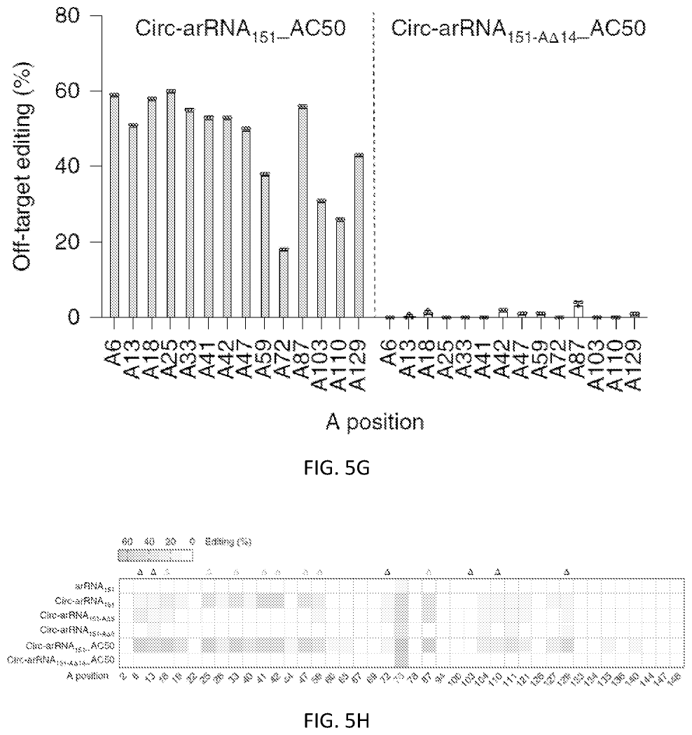

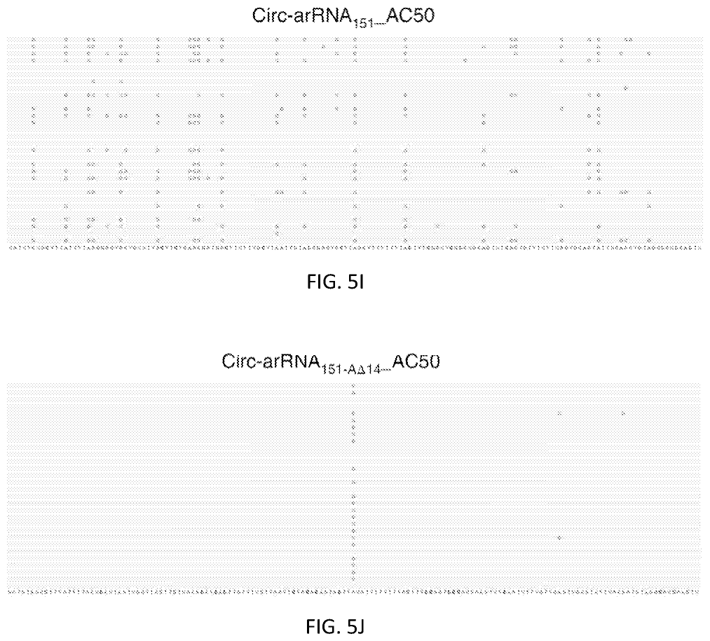

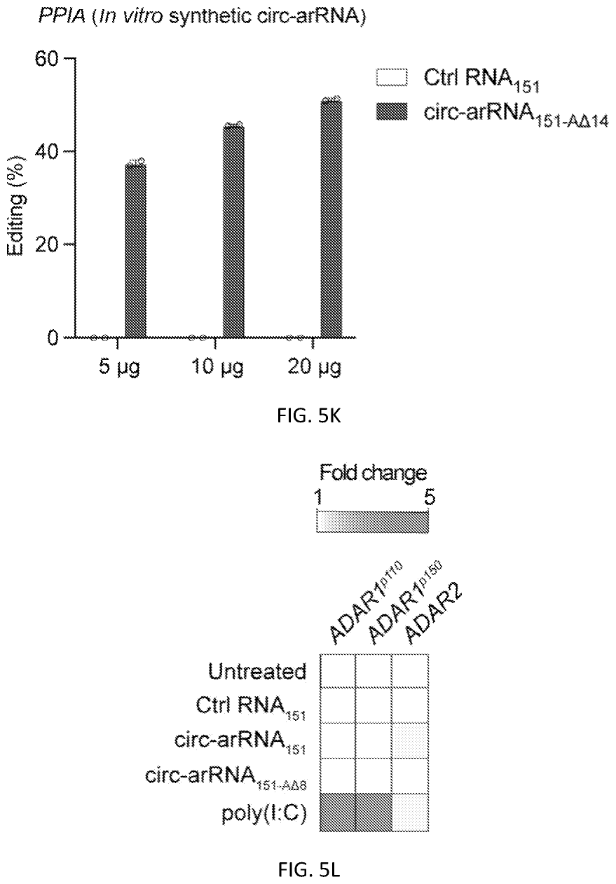

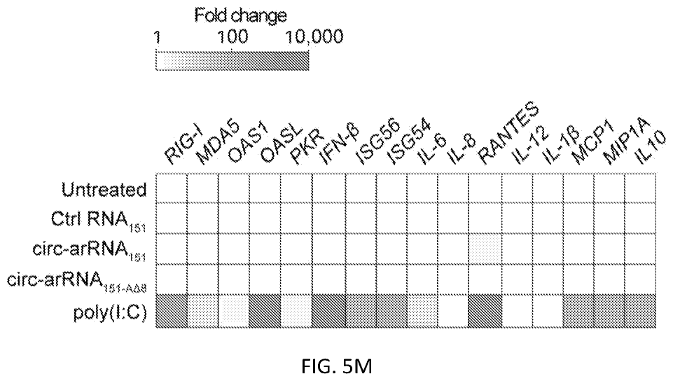

As used herein, the term “bulge” refers to an asymmetric bubble region in a nucleic acid duplex formed due to one or more unpaired nucleotides (e.g., a non-target adenosine) in one strand of the nucleic acid duplex. A bulge described herein may have a fully unpaired region in one strand that does not have any corresponding complementary region in the opposite strand. Alternatively, a bulge described herein may be formed by two non-complementary regions (one in each strand) having different number of nucleotides, which may further contain mismatched nucleotides that do not form Watson-Crick base pairs. The longer of the two non-complementary regions has at least one nucleotide (e.g., a non-target adenosine) that is not paired with any nucleotides in the non-complementary region of the opposite strand, i.e., the opposite strand comprises nucleic acid sequence(s) complementary to nucleic acid sequence(s) flanking the bulge but the opposite strand does not contain at least one nucleotide opposite a nucleotide (e.g., a non-target adenosine) in the bulge. A “bulge” described herein does not encompass a fully mispaired region of nucleotides located within one strand of a nucleic acid duplex, i.e., the opposite strand contains a nucleotide that is non-complementary for each of the nucleotides in the bulge, which results in a symmetric bubble in the nucleic acid duplex. In some examples, the bulge contain 1, 2, 3, 4, 5, or greater than 5 nucleotides in the strand having the unpaired nucleotides. For example, the duplex shown in the schematic of FIG. 5A has a bulge containing 1 nucleotide, which is a non-target adenosine, in the mRNA strand (top strand) and no corresponding nucleotide in the linear arRNAdC strand (bottom strand).

When a first nucleic acid strand and a second nucleic acid strand form a double-stranded nucleic acid region, a first nucleoside in the first nucleic acid strand that base-pair with a second nucleoside in the second nucleic acid strand are described herein as being “opposite” with respect to each other, or “corresponding” to each other, i.e., the first nucleoside is opposite to the second nucleoside, and the second nucleoside is opposite to the first nucleoside.

The terms “polynucleotide,” “nucleic acid,” “nucleotide sequence,” and “nucleic acid sequence” are used interchangeably. They refer to a polymeric form of nucleotides of any length, either deoxyribonucleotides or ribonucleotides, or analogs thereof. It will be understood by one of ordinary skill in the art that uracil and thymine can both be represented by ‘t’, instead of ‘u’ for uracil and ‘t’ for thymine; in the context of a ribonucleic acid (RNA), it will be understood that ‘t’ is used to represent uracil unless otherwise indicated.

The terms “deaminase-recruiting RNA,” “dRNA,” “ADAR-recruiting RNA” and “arRNA” are used herein interchangeably to refer to an engineered RNA capable of recruiting an ADAR to deaminate a target adenosine in an RNA.

The terms “Group I intron” and “Group I catalytic intron” are used interchangeably to refer to a self-splicing ribozyme that can catalyze its own excision from an RNA precursor. Group I introns comprise two fragments, the 5′ catalytic Group I intron fragment and the 3′ catalytic Group I intron fragment, which retain their folding and catalytic function (i.e., self-splicing activity). In its native environment, the 5′ catalytic Group I intron fragment is flanked at its 5′ end by a 5′ exon, which comprises a 5′ exon sequence that is recognized by the 5′ catalytic Group I intron fragment; and the 3′ catalytic Group I intron fragment is flanked at its 3′ end by a 3′ exon, which comprises a 3′ exon sequence that is recognized by the 3′ catalytic Group I intron fragment. The terms “5′ exon sequence” and “3′ exon sequence” used herein are labeled according to the order of the exons with respect to the Group I intron in its natural environment.

The terms “adenine,” “guanine,” “cytosine,” “thymine,” “uracil” and “hypoxanthine” as used herein refer to the nucleobases as such. The terms “adenosine,” “guanosine,” “cytidine,” “thymidine,” “uridine” and “inosine,” refer to the nucleobases linked to the ribose or deoxyribose sugar moiety. The term “nucleoside” refers to the nucleobase linked to the ribose or deoxyribose. The term “nucleotide” refers to the respective nucleobase-ribosyl-phosphate or nucleobase-deoxyribosyl-phosphate. Sometimes the terms adenosine and adenine (with the abbreviation, “A”), guanosine and guanine (with the abbreviation, “G”), cytosine and cytidine (with the abbreviation, “C”), uracil and uridine (with the abbreviation, “U”), thymine and thymidine (with the abbreviation, “T”), inosine and hypo-xanthine (with the abbreviation, “I”), are used interchangeably to refer to the corresponding nucleobase, nucleoside or nucleotide. Sometimes the terms nucleobase, nucleoside and nucleotide are used interchangeably, unless the context clearly requires differently.

The term “functional protein” refers to a naturally-occurring protein, functional variants thereof, or an engineered derivative thereof that is functional in treating a genetic disease or condition. The disease or condition may be caused in whole or in part by a change, such as a mutation, in the wildtype, naturally-occurring protein corresponding to the functional protein.

The term “functional variant” of a reference protein refers to a variant polypeptide derived from the reference protein or a portion thereof, and the variant has substantially the same activity (e.g., binding to a target or enzymatic activity) as the reference protein. “Substantially the same activity” means an activity level that is at least about any one of 20%, 30%, 40%, 50%, 60%, 70%, 80%, 90%, or more as the activity of the reference protein.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

The term “introducing” or “introduction” used herein means delivering one or more polynucleotides, such as dRNAs or one or more constructs including vectors as described herein, one or more transcripts thereof, to a host cell. The invention serves as a basic platform for enabling targeted editing of RNA, for example, pre-messenger RNA, a messenger RNA, a ribosomal RNA, a transfer RNA, a long non-coding RNA and a small RNA (such as miRNA). The methods of the present application can employ many delivery systems, including but not limited to, viral, liposome, electroporation, microinjection and conjugation, to achieve the introduction of the dRNA or construct as described herein into a host cell. Conventional viral and non-viral based gene transfer methods can be used to introduce nucleic acids into mammalian cells or target tissues. Such methods can be used to administer nucleic acids encoding dRNA of the present application to cells in culture, or in a host organism. Non-viral vector delivery systems include DNA plasmids, RNA (e.g. a transcript of a construct described herein), naked nucleic acid, and nucleic acid complexed with a delivery vehicle, such as a liposome. Viral vector delivery systems include DNA and RNA viruses, which have either episomal or integrated genomes for delivery to the host cell.

In the context of the present application, “target RNA” refers to an RNA sequence to which a deaminase-recruiting RNA sequence is designed to have perfect complementarity or substantial complementarity, and hybridization between the target sequence and the dRNA forms a double stranded RNA (dsRNA) region containing a target adenosine, which recruits an adenosine deaminase acting on RNA (ADAR) that deaminates the target adenosine. In some embodiments, the ADAR is naturally present in a host cell, such as a eukaryotic cell (such as a mammalian cell, e.g., a human cell). In some embodiments, the ADAR is introduced into the host cell.

As used herein, “operably linked,” when referring to a first nucleic acid sequence that is operably linked with a second nucleic acid sequence, means a situation when the first nucleic acid sequence is placed in a functional relationship with the second nucleic acid sequence. For instance, a promoter is operably linked to a coding sequence if the promoter effects the transcription of the coding sequence. Likewise, the coding sequence of a signal peptide is operably linked to the coding sequence of a polypeptide if the signal peptide effects the extracellular secretion of that polypeptide. Generally, operably linked nucleic acid sequences are contiguous and, where necessary to join two protein coding regions, the open reading frames are aligned.

As used herein, “connect” refers to linking of nucleic acid sequences, either directly or indirectly, for example, via an intervening nucleic acid sequence.

As used herein, “complementarity” refers to the ability of a nucleic acid to form hydrogen bond(s) with another nucleic acid by traditional Watson-Crick base-pairing. A percent complementarity indicates the percentage of residues in a nucleic acid molecule which can form hydrogen bonds (i.e., Watson-Crick base pairing) with a second nucleic acid (e.g., about 5, 6, 7, 8, 9, 10 out of 10, being about 50%, 60%, 70%, 80%, 90%, and 100% complementary respectively). “Perfectly complementary” means that all the contiguous residues of a nucleic acid sequence form hydrogen bonds with the same number of contiguous residues in a second nucleic acid sequence. “Substantially complementary” as used herein refers to a degree of complementarity that is at least about any one of 70%, 75%, 80%, 85%, 90%, 95%, 97%, 98%, 99%, or 100% over a region of about 40, 50, 60, 70, 80, 100, 150, 200, 250 or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology—Hybridization With Nucleic Acid Probes Part I, Second Chapter “Overview of principles of hybridization and the strategy of nucleic acid probe assay,” Elsevier, N,Y.

“Hybridization” or “hybridize” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. A sequence capable of hybridizing with a given sequence is referred to as the “complement” of the given sequence.

A “subject,” “patient,” or “individual” includes a mammal, such as a human or other animal, and typically is human. In some embodiments, the subject, e.g., patient, to whom the therapeutic agents and compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent, a dog, a cat, a farm animal, such as a cow or a horse, etc.

As used herein, the term “treatment” refers to clinical intervention designed to have beneficial and desired effects to the natural course of the individual or cell being treated during the course of clinical pathology. For the purpose of this disclosure, desirable effects of treatment include, without limitation, decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, increasing cancer cell-killing, decreasing symptoms resulting from the disease, preventing spread of diseases, preventing recurrence of disease, increasing the quality of life of those suffering from the disease, decreasing the dose of other medications required to treat the disease, delaying the progression of the disease, and/or prolonging survival of individuals.

As used herein, the term “effective amount” or “therapeutically effective amount” of a substance is at least the minimum concentration required to effect a measurable improvement or prevention of a particular disorder. An effective amount herein may vary according to factors such as the disease state, age, sex, and weight of the patient, and the ability of the substance to elicit a desired response in the individual. An effective amount is also one in which any toxic or detrimental effects of the treatment are outweighed by the therapeutically beneficial effects. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer. An effective amount can be administered in one or more administrations. For purposes of this disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein the term “wild type” is a term of the art understood by skilled persons and means the typical form of an organism, strain, gene or characteristic as it occurs in nature as distinguished from mutant or variant forms.

A “host cell” as described herein refers to any cell type that can be used as a host cell provided it can be modified as described herein. For example, the host cell may be a host cell with endogenously expressed adenosine deaminase acting on RNA (ADAR), or may be a host cell into which an adenosine deaminase acting on RNA (ADAR) is introduced by a known method in the art. For example, the host cell may be a prokaryotic cell, a eukaryotic cell or a plant cell. In some embodiments, the host cell is derived from a pre-established cell line, such as mammalian cell lines including human cell lines or non-human cell lines. In some embodiments, the host cell is derived from an individual, such as a human individual.

A “recombinant AAV vector (rAAV vector)” refers to a polynucleotide vector comprising one or more heterologous sequences (i.e., nucleic acid sequence not of AAV origin) that are flanked by at least one, and in embodiments two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, particularly an AAV particle. A rAAV vector can be packaged into an AAV virus capsid to generate a “recombinant adeno-associated viral particle (rAAV particle)”.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

As such, polynucleotides encoding peptides or polypeptides containing substitutions, insertions and/or additions, deletions and covalent modifications with respect to reference sequences, in particular, the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, a “carrier” includes pharmaceutically acceptable carriers, excipients, or stabilizers that are nontoxic to the cell or mammal being exposed thereto at the dosages and concentrations employed. Often the physiologically acceptable carrier is an aqueous pH buffered solution. Non-limiting examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or condition. In some embodiments, the manufacture or kit is promoted, distributed, or sold as a unit for performing the methods described herein.

As used herein, the terms “including,” “containing,” and “comprising” are used in their open, non-limiting sense. It is also understood that aspects and embodiments of the present application described herein may include “consisting” and/or “consisting essentially of” aspects and embodiments.

It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Methods of RNA Editing

Provided herein are methods for editing a target RNA in a host cell using a deaminase-recruiting RNA (dRNA) comprising a targeting RNA sequence having deletion of one or more nucleosides opposite a region comprising a non-target adenosine in a target RNA and/or comprising one or more linker nucleic acid sequences flanking the 5′ and/or 3′ end of the targeting RNA sequence, wherein the dRNA is capable of recruiting an adenosine deaminase acting on RNA (ADAR). The dRNA may be any one of the dRNAs described in section III (“dRNAs, constructs and libraries”) below. In some embodiments, the dRNA is linear. In some embodiments, the dRNA is circular. In some embodiments, the dRNA is a linear RNA capable of forming a circular RNA. In some embodiments, the method uses a construct comprising a nucleic acid sequence encoding the dRNA. The construct may be any one of the constructs described in section III below.

In some embodiments, there is provided a method for editing a target adenosine in a target RNA in a host cell, comprising introducing a dRNA or a construct comprising a nucleic acid sequence encoding the dRNA into the host cell, wherein: (1) the dRNA comprises a targeting RNA sequence that is capable of hybridizing to the target RNA to form a duplex RNA, wherein the duplex RNA comprises a bulge comprising a non-target adenosine in the target RNA; and (2) the dRNA is capable of recruiting an ADAR. In some embodiments, the duplex RNA comprises a bulge at each non-target adenosine in the target RNA. In some embodiments, the targeting RNA sequence is complementary to the target RNA except for lacking one or more nucleotides opposite non-target adenosines in the target RNA. In some embodiments, the dRNA is linear. In some embodiments, the dRNA is circular. In some embodiments, the dRNA is a linear RNA capable of forming a circular RNA. In some embodiments, the method does not comprise introducing any protein or construct comprising a nucleic acid encoding a protein (e.g., Cas, ADAR or a fusion protein of ADAR and Cas) to the host cell.

In some embodiments, there is provided a method for editing a target adenosine in a target RNA in a host cell, comprising introducing a dRNA or a construct comprising a nucleic acid sequence encoding the dRNA into the host cell, wherein: (1) the dRNA comprises a targeting RNA sequence that is capable of hybridizing to the target RNA, wherein the dRNA comprises a linker nucleic acid sequence flanking an end of the targeting RNA sequence, wherein the linker nucleic acid sequence does not substantially form any secondary structure with any part of the dRNA; (2) the dRNA is capable of recruiting an ADAR; and (3) the dRNA is a circular RNA or a linear RNA capable of forming a circular RNA. In some embodiments, the linker nucleic acid sequence is about 5 nt to about 500 nt long, such as about 50 nt to 200 nt long. In some embodiments, the linker nucleic acid sequence comprises a polyadenosine (polyA), polyguanosine (polyG), or polycytosine (polyC) sequence. In some embodiments, at least 50% of the linker nucleic acid sequence comprises adenosine. In some embodiments, the linker nucleic acid sequence comprises a dinucleotide repeat sequence, such as (AT)_{n,} wherein n is an integer greater or equal to 3. In some embodiments, the linker nucleic acid sequence comprises SEQ ID NO: 22. In some embodiments, the method does not comprise introducing any protein or construct comprising a nucleic acid encoding a protein (e.g., Cas, ADAR or a fusion protein of ADAR and Cas) to the host cell.

In some embodiments, there is provided a method for editing a target adenosine in a target RNA in a host cell, comprising introducing a dRNA or a construct comprising a nucleic acid sequence encoding the dRNA into the host cell, wherein: (1) the dRNA comprises a targeting RNA sequence that is capable of hybridizing to the target RNA, wherein the dRNA comprises a first linker nucleic acid sequence flanking the 5′ end of the targeting RNA sequence and a second linker nucleic acid sequence flanking the 3′ end of the targeting RNA sequence, wherein the linker nucleic acid sequence does not substantially form any secondary structure with any part of the dRNA; (2) the dRNA is capable of recruiting an ADAR; and (3) the dRNA is a circular RNA or a linear RNA capable of forming a circular RNA. In some embodiments, the first linker nucleic acid sequence and/or the second linker nucleic acid sequence is about 5 nt to about 500 nt long, such as about 50 nt to 200 nt long. In some embodiments, the first linker nucleic acid sequence is identical to the second linker nucleic acid sequence. In some embodiments, the first linker nucleic acid sequence is different from the second linker nucleic acid sequence. In some embodiments, In some embodiments, the first linker nucleic acid sequence and/or the second linker nucleic acid sequence comprises a polyadenosine (polyA), polyguanosine (polyG), or polycytosine (polyC) sequence. In some embodiments, the first linker nucleic acid sequence and/or the second linker nucleic acid sequence comprises a dinucleotide repeat sequence, such as (AT)_{n,} wherein n is an integer greater or equal to 3. In some embodiments, the first linker nucleic acid sequence and/or the second linker nucleic acid sequence comprises SEQ ID NO: 22. In some embodiments, the dRNA is a circular RNA, and wherein the linker nucleic acid sequence connects the 5′ end of the targeting RNA sequence and the 3′ end of the targeting RNA sequence. In some embodiments, the method does not comprise introducing any protein or construct comprising a nucleic acid encoding a protein (e.g., Cas, ADAR or a fusion protein of ADAR and Cas) to the host cell.

In some embodiments, there is provided a method for editing a target adenosine in a target RNA in a host cell, comprising introducing a dRNA or a construct comprising a nucleic acid sequence encoding the dRNA into the host cell, wherein: (1) the dRNA comprises a targeting RNA sequence that is capable of hybridizing to the target RNA to form a duplex RNA, wherein the duplex RNA comprises a bulge comprising a non-target adenosine in the target RNA, wherein the dRNA comprises a linker nucleic acid sequence flanking an end of the targeting RNA sequence, wherein the linker nucleic acid sequence does not substantially form any secondary structure with any part of the dRNA; (2) the dRNA is capable of recruiting an ADAR; and (3) the dRNA is a circular RNA or a linear RNA capable of forming a circular RNA. In some embodiments, the duplex RNA comprises a bulge at each non-target adenosine in the target RNA. In some embodiments, the targeting RNA sequence is complementary to the target RNA except for lacking one or more nucleotides opposite non-target adenosines in the target RNA. In some embodiments, the linker nucleic acid sequence is about 5 nt to about 500 nt long, such as about 50 nt to 200 nt long. In some embodiments, the linker nucleic acid sequence comprises a polyadenosine (polyA), polyguanosine (polyG), or polycytosine (polyC) sequence. In some embodiments, at least 50% of the linker nucleic acid sequence comprises adenosine. In some embodiments, the linker nucleic acid sequence comprises a dinucleotide repeat sequence, such as (AT)_{n,} wherein n is an integer greater or equal to 3. In some embodiments, the linker nucleic acid sequence comprises SEQ ID NO: 22. In some embodiments, the dRNA comprises a first linker nucleic acid sequence flanking the 5′ end of the targeting RNA sequence and a second linker nucleic acid sequence flanking the 3′ end of the targeting RNA sequence. In some embodiments, the dRNA comprises a first linker nucleic acid sequence flanking the 5′ end of the targeting RNA sequence and a second linker nucleic acid sequence replacing the 3′ end of the targeting RNA sequence. In some embodiments, the dRNA comprises a first linker nucleic acid sequence replacing the 5′ end of the targeting RNA sequence and a second linker nucleic acid sequence flanking the 3′ end of the targeting RNA sequence. In some embodiments, the dRNA comprises a first linker nucleic acid sequence replacing the 5′ end of the targeting RNA sequence and a second linker nucleic acid sequence replacing the 3′ end of the targeting RNA sequence. In some embodiments, the first linker nucleic acid sequence is identical to the second linker nucleic acid sequence. In some embodiments, the first linker nucleic acid sequence is different from the second linker nucleic acid sequence. In some embodiments, the dRNA is a circular RNA, and wherein the linker nucleic acid sequence connects the 5′ end of the targeting RNA sequence and the 3′ end of the targeting RNA sequence. In some embodiments, the method does not comprise introducing any protein or construct comprising a nucleic acid encoding a protein (e.g., Cas, ADAR or a fusion protein of ADAR and Cas) to the host cell.

In some embodiments, there is provided a method for editing a target adenosine in a target RNA in a host cell, comprising introducing a circular dRNA or a construct comprising a nucleic acid sequence encoding the circular dRNA into the host cell, wherein: (1) the circular dRNA comprises a targeting RNA sequence that is capable of hybridizing to the target RNA to form a duplex RNA, wherein the duplex RNA comprises a bulge comprising a non-target adenosine in the target RNA; and (2) the circular dRNA is capable of recruiting an ADAR. In some embodiments, the duplex RNA comprises a bulge at each non-target adenosine in the target RNA. In some embodiments, the targeting RNA sequence is complementary to the target RNA except for lacking one or more nucleotides opposite non-target adenosines in the target RNA. In some embodiments, the circular dRNA further comprises a linker nucleic acid sequence flanking an end of the targeting RNA sequence, wherein the linker nucleic acid sequence does not substantially form any secondary structure with any part of the dRNA. In some embodiments, the circular dRNA further comprises a linker nucleic acid sequence replacing an end of the targeting RNA sequence, wherein the linker nucleic acid sequence does not substantially form any secondary structure with any part of the dRNA. In some embodiments, the linker nucleic acid sequence is about 5 nt to about 500 nt long, such as about 50 nt to 200 nt long. In some embodiments, the linker nucleic acid sequence comprises SEQ ID NO: 22. In some embodiments, the linker nucleic acid sequence connects the 5′ end of the targeting RNA sequence and the 3′ end of the targeting RNA sequence. In some embodiments, the dRNA comprises a first linker nucleic acid sequence flanking the 5′ end of the targeting RNA sequence and a second linker nucleic acid sequence flanking the 3′ end of the targeting RNA sequence. In some embodiments, the dRNA comprises a first linker nucleic acid sequence flanking the 5′ end of the targeting RNA sequence and a second linker nucleic acid sequence replacing the 3′ end of the targeting RNA sequence. In some embodiments, the dRNA comprises a first linker nucleic acid sequence replacing the 5′ end of the targeting RNA sequence and a second linker nucleic acid sequence flanking the 3′ end of the targeting RNA sequence. In some embodiments, the dRNA comprises a first linker nucleic acid sequence replacing the 5′ end of the targeting RNA sequence and a second linker nucleic acid sequence replacing the 3′ end of the targeting RNA sequence. In some embodiments, the dRNA further comprises a 3′ exon sequence recognizable by a 3′ catalytic Group I intron fragment flanking the 5′ end of the targeting RNA sequence, and a 5′ exon sequence recognizable by a 5′ catalytic Group I intron fragment flanking the 3′ end of the targeting RNA sequence. In some embodiments, the dRNA further comprises a 3′ ligation sequence and a 5′ ligation sequence. In some embodiments, the method does not comprise introducing any protein or construct comprising a nucleic acid encoding a protein (e.g., Cas, ADAR or a fusion protein of ADAR and Cas) to the host cell.

In some embodiments, there is provided a method for editing a target adenosine in a target RNA in a host cell, comprising introducing into the host cell (a) a dRNA or a construct comprising a nucleic acid sequence encoding the dRNA and (b) an ADAR or a construct comprising a nucleic acid encoding the ADAR, wherein: (1) the dRNA comprises a targeting RNA sequence that is capable of hybridizing to the target RNA to form a duplex RNA, wherein the duplex RNA comprises a bulge comprising a non-target adenosine in the target RNA; and (2) the dRNA is capable of recruiting the ADAR. In some embodiments, the duplex RNA comprises a bulge at each non-target adenosine in the target RNA. In some embodiments, the targeting RNA sequence is complementary to the target RNA except for lacking one or more nucleotides opposite non-target adenosines in the target RNA. In some embodiments, the ADAR is an endogenously encoded ADAR of the host cell, wherein introduction of the ADAR comprises over-expressing the ADAR in the host cell. In some embodiments, the ADAR is exogenous to the host cell. In some embodiments, the construct comprising a nucleic acid encoding the ADAR is a vector, such as a plasmid, or a viral vector (e.g., an AAV such as a scAAV).

In some embodiments, there is provided a method for editing a target adenosine in a target RNA in a host cell, comprising introducing into the host cell (a) a dRNA or a construct comprising a nucleic acid sequence encoding the dRNA and (b) an ADAR or a construct comprising a nucleic acid encoding the ADAR, wherein: (1) the dRNA comprises a targeting RNA sequence that is capable of hybridizing to the target RNA, wherein the dRNA comprises a linker nucleic acid sequence flanking an end of the targeting RNA sequence, wherein the linker nucleic acid sequence does not substantially form any secondary structure with any part of the dRNA; (2) (2) the dRNA is capable of recruiting the ADAR; and (3) the dRNA is a circular RNA or a linear RNA capable of forming a circular RNA. In some embodiments, the linker nucleic acid sequence is about 5 nt to about 500 nt long, such as about 50 nt to 200 nt long. In some embodiments, the linker nucleic acid sequence comprises a polyadenosine (polyA), polyguanosine (polyG), or polycytosine (polyC) sequence. In some embodiments, at least 50% of the linker nucleic acid sequence comprises adenosine. In some embodiments, the linker nucleic acid sequence comprises a dinucleotide repeat sequence, such as (AT)_{n,} wherein n is an integer greater or equal to 3. In some embodiments, the linker nucleic acid sequence comprises SEQ ID NO: 22. In some embodiments, the dRNA comprises a first linker nucleic acid sequence flanking the 5′ end of the targeting RNA sequence and a second linker nucleic acid sequence flanking the 3′ end of the targeting RNA sequence. In some embodiments, the dRNA comprises a first linker nucleic acid sequence flanking the 5′ end of the targeting RNA sequence and a second linker nucleic acid sequence replacing the 3′ end of the targeting RNA sequence. In some embodiments, the dRNA comprises a first linker nucleic acid sequence replacing the 5′ end of the targeting RNA sequence and a second linker nucleic acid sequence flanking the 3′ end of the targeting RNA sequence. In some embodiments, the dRNA comprises a first linker nucleic acid sequence replacing the 5′ end of the targeting RNA sequence and a second linker nucleic acid sequence replacing the 3′ end of the targeting RNA sequence. In some embodiments, the ADAR is an endogenously encoded ADAR of the host cell, wherein introduction of the ADAR comprises over-expressing the ADAR in the host cell. In some embodiments, the ADAR is exogenous to the host cell. In some embodiments, the construct comprising a nucleic acid encoding the ADAR is a vector, such as a plasmid, or a viral vector (e.g., an AAV such as a scAAV).

In some embodiments, there is provided a method for editing a target adenosine in a target RNA in a host cell, comprising introducing into the host cell (a) a dRNA or a construct comprising a nucleic acid sequence encoding the dRNA and (b) an ADAR or a construct comprising a nucleic acid encoding the ADAR, wherein: (1) the dRNA comprises a targeting RNA sequence that is capable of hybridizing to the target RNA to form a duplex RNA, wherein the duplex RNA comprises a bulge comprising a non-target adenosine in the target RNA, wherein the dRNA comprises a linker nucleic acid sequence flanking an end of the targeting RNA sequence, wherein the linker nucleic acid sequence does not substantially form any secondary structure with any part of the dRNA; (2) the dRNA is capable of recruiting the ADAR; and (3) the dRNA is a circular RNA or a linear RNA capable of forming a circular RNA. In some embodiments, the duplex RNA comprises a bulge at each non-target adenosine in the target RNA. In some embodiments, the targeting RNA sequence is complementary to the target RNA except for lacking one or more nucleotides opposite non-target adenosines in the target RNA. In some embodiments, the linker nucleic acid sequence is about 5 nt to about 500 nt long, such as about 50 nt to 200 nt long. In some embodiments, the linker nucleic acid sequence comprises a polyadenosine (polyA), polyguanosine (polyG), or polycytosine (polyC) sequence. In some embodiments, at least 50% of the linker nucleic acid sequence comprises adenosine. In some embodiments, the linker nucleic acid sequence comprises a dinucleotide repeat sequence, such as (AT)_{n,} wherein n is an integer greater or equal to 3. In some embodiments, the linker nucleic acid sequence comprises SEQ ID NO: 22. In some embodiments, the dRNA comprises a first linker nucleic acid sequence flanking the 5′ end of the targeting RNA sequence and a second linker nucleic acid sequence flanking the 3′ end of the targeting RNA sequence. In some embodiments, the dRNA comprises a first linker nucleic acid sequence flanking the 5′ end of the targeting RNA sequence and a second linker nucleic acid sequence replacing the 3′ end of the targeting RNA sequence. In some embodiments, the dRNA comprises a first linker nucleic acid sequence replacing the 5′ end of the targeting RNA sequence and a second linker nucleic acid sequence flanking the 3′ end of the targeting RNA sequence. In some embodiments, the dRNA comprises a first linker nucleic acid sequence replacing the 5′ end of the targeting RNA sequence and a second linker nucleic acid sequence replacing the 3′ end of the targeting RNA sequence. In some embodiments, the dRNA is a circular RNA, and wherein the linker nucleic acid sequence connects the 5′ end of the targeting RNA sequence and the 3′ end of the targeting RNA sequence. In some embodiments, the ADAR is an endogenously encoded ADAR of the host cell, wherein introduction of the ADAR comprises over-expressing the ADAR in the host cell. In some embodiments, the ADAR is exogenous to the host cell. In some embodiments, the construct comprising a nucleic acid encoding the ADAR is a vector, such as a plasmid, or a viral vector (e.g., an AAV such as a scAAV).

In some embodiments, the present invention provides the method for editing a target adenosine in a target RNA as disclosed herein, wherein the dRNA or the construct comprising a nucleic acid sequence encoding the dRNA edits the target adenosine in the target RNA in a dose dependent manner.

In some embodiments, there is provided a method for reducing editing of a non-target adenosine (also referred herein as “bystander editing”) in a target RNA in a host cell, comprising: introducing into the host cell a dRNA or a construct comprising a nucleic acid sequence encoding the dRNA, wherein: (1) the dRNA comprises a targeting RNA sequence that is capable of hybridizing to the target RNA to form a duplex RNA, wherein the duplex RNA comprises a bulge comprising the non-target adenosine in the target RNA; and (2) the dRNA is capable of recruiting an ADAR; wherein the editing rate of the non-target adenosine is reduced compared to a method using a dRNA comprising a targeting RNA sequence that is complementary to the target RNA. In some embodiments, the duplex RNA comprises a bulge at each non-target adenosine in the target RNA. In some embodiments, the targeting RNA sequence is complementary to the target RNA except for lacking one or more nucleotides opposite non-target adenosines in the target RNA. In some embodiments, the targeting RNA sequence is complementary to the target RNA except for lacking two or more consecutive nucleotides opposite non-target adenosines in the target RNA. In some embodiments, the dRNA comprises a linker nucleic acid sequence flanking an end of the targeting RNA sequence, wherein the linker nucleic acid sequence does not substantially form any secondary structure with any part of the dRNA. In some embodiments, the dRNA comprises a linker nucleic acid sequence replacing an end of the targeting RNA sequence, wherein the linker nucleic acid sequence does not substantially form any secondary structure with any part of the dRNA. In some embodiments, the linker nucleic acid sequence is about 5 nt to about 500 nt long, such as about 50 nt to 200 nt long. In some embodiments, the linker nucleic acid sequence comprises a polyadenosine (polyA), polyguanosine (polyG), or polycytosine (polyC) sequence. In some embodiments, at least 50% of the linker nucleic acid sequence comprises adenosine. In some embodiments, the linker nucleic acid sequence comprises a dinucleotide repeat sequence, such as (AT)_{n,} wherein n is an integer greater or equal to 3. In some embodiments, the linker nucleic acid sequence comprises SEQ ID NO: 22. In some embodiments, the dRNA comprises a first linker nucleic acid sequence flanking the 5′ end of the targeting RNA sequence and a second linker nucleic acid sequence flanking the 3′ end of the targeting RNA sequence. In some embodiments, the dRNA is a circular RNA, and wherein the linker nucleic acid sequence connects the 5′ end of the targeting RNA sequence and the 3′ end of the targeting RNA sequence. In some embodiments, the editing rate of the non-target adenosine is reduced by at least about 20%, 30%, 50%, 60%, 70%, 80%, 90%, 95%, or more compared to a method using a dRNA comprising a targeting RNA sequence that is complementary to the target RNA.

In some embodiments, there is provided a method for increasing editing efficiency of a target adenosine in a target RNA in a host cell, comprising: introducing into the host cell a dRNA or a construct comprising a nucleic acid sequence encoding the dRNA, wherein: (1) the dRNA comprises a targeting RNA sequence that is capable of hybridizing to the target RNA, wherein the dRNA comprises a linker nucleic acid sequence flanking an end of the targeting RNA sequence, wherein the linker nucleic acid sequence does not substantially form any secondary structure with any part of the dRNA; and (2) the dRNA is capable of recruiting an ADAR; wherein the editing efficiency of the target adenosine is increased compared to a method using a dRNA that does not comprise the linker nucleic acid sequence. In some embodiments, the linker nucleic acid sequence is about 5 nt to about 500 nt long, such as about 50 nt to 200 nt long. In some embodiments, the linker nucleic acid sequence comprises a polyadenosine (polyA), polyguanosine (polyG), or polycytosine (polyC) sequence. In some embodiments, the linker nucleic acid sequence comprises a dinucleotide repeat sequence, such as (AT)_{n,} wherein n is an integer greater or equal to 3. In some embodiments, the linker nucleic acid sequence comprises SEQ ID NO: 22. In some embodiments, the dRNA comprises a first linker nucleic acid sequence flanking the 5′ end of the targeting RNA sequence and a second linker nucleic acid sequence flanking the 3′ end of the targeting RNA sequence. In some embodiments, the dRNA is a circular RNA, and wherein the linker nucleic acid sequence connects the 5′ end of the targeting RNA sequence and the 3′ end of the targeting RNA sequence. In some embodiments, the dRNA comprises a targeting RNA sequence that is capable of hybridizing to the target RNA to form a duplex RNA, wherein the duplex RNA comprises a bulge comprising the non-target adenosine in the target RNA. In some embodiments, the duplex RNA comprises a bulge at each non-target adenosine in the target RNA. In some embodiments, the targeting RNA sequence is complementary to the target RNA except for lacking one or more nucleotides opposite non-target adenosines in the target RNA. In some embodiments, the editing efficiency of the target adenosine is increased by at least about 50%, 2×, 3×, 4×, 5×, 6×, 7×, 8×, 9×, 10× or more compared to a method using a dRNA that does not comprise the linker nucleic acid sequence.

In one aspect, the present application provides a method for editing a plurality of target RNAs (e.g., at least about 2, 3, 4, 5, 10, 20, 50, 100, 1000 or more) in host cells by introducing a plurality of the dRNAs, or one or more constructs encoding the dRNAs, into the host cells.

In some embodiments, the host cell is a prokaryotic cell. In some embodiments, the host cell is a eukaryotic cell. In some embodiments, the host cell is a mammalian cell. In some embodiments, the host cell is a human cell. In some embodiments, the host cell is a murine cell. In some embodiments, the host cell is a plant cell or a fungal cell.

In some embodiments, the host cell is a cell line, such as HEK293T, HT29, A549, HepG2, RD, SF268, SW13 and HeLa cell. In some embodiments, the host cell is a primary cell, such as fibroblast, epithelial, or immune cell. In some embodiments, the host cell is a T cell. In some embodiments, the host cell is a post-mitosis cell. In some embodiments, the host cell is a cell of the central nervous system (CNS), such as a brain cell, e.g., a cerebellum cell.

In some embodiments, the ADAR is endogenous to the host cell. In some embodiments, the adenosine deaminase acting on RNA (ADAR) is naturally or endogenously present in the host cell, for example, naturally or endogenously present in the eukaryotic cell. In some embodiments, the ADAR is endogenously expressed by the host cell. In some embodiments, the ADAR is exogenously introduced into the host cell. In some embodiments, the ADAR is ADAR1 and/or ADAR2. In some embodiments, the ADAR is one or more ADARs selected from the group consisting of hADAR1, hADAR2, mouse ADAR1 and ADAR2. In some embodiments, the ADAR is ADAR1, such as p110 isoform of ADAR1 (“ADAR1^{p110}”) and/or p150 isoform of ADAR1 (“ADAR1^{p150}”). In some embodiments, the ADAR is ADAR2. In some embodiments, the ADAR is an ADAR2 expressed by the host cell, e.g., ADAR2 expressed by cerebellum cells.

In some embodiments, the ADAR is an ADAR exogenous to the host cell. In some embodiments, the ADAR is a hyperactive mutant of a naturally occurring ADAR. In some embodiments, the ADAR is ADAR1 comprising an E1008Q mutation. In some embodiments, the ADAR is not a fusion protein comprising a binding domain. In some embodiments, the ADAR does not comprise an engineered double-strand nucleic acid-binding domain. In some embodiments, the ADAR does not comprise a MCP domain that binds to MS2 hairpin that is fused to the complementary RNA sequence in the dRNA.

In some embodiments, the host cell has high expression level of ADAR1 (such as ADAR1^{p110} and/or ADAR1^{p150}) e.g., at least about any one of 10%, 20%, 50%, 100%, 2×, 3×, 5×, or more relative to the protein expression level of β-tubulin. In some embodiments, the host cell has high expression level of ADAR2, e.g., at least about any one of 10%, 20%, 50%, 100%, 2×, 3×, 5×, or more relative to the protein expression level of β-tubulin. In some embodiments, the host cell has low expression level of ADAR3, e.g., no more than about any one of 5×, 3×, 2×, 100%, 50%, 20% or less relative to the protein expression level of β-tubulin.

In certain embodiments, the method further comprises introducing an inhibitor of ADAR3 to the host cell. In some embodiments, the inhibitor of ADAR3 is an RNAi against ADAR3, such as a shRNA against ADAR3 or a siRNA against ADAR3. In some embodiments, the method further comprises introducing a stimulator of interferon to the host cell. In some embodiments, the ADAR is inducible by interferon, for example, the ADAR is ADAR^{p150}. In some embodiments, the stimulator of interferon is IFNα. In some embodiments, the inhibitor of ADAR3 and/or the stimulator of interferon are encoded by the same construct (e.g., vector) that encodes the dRNA.

In certain embodiments, the method does not induce immune response, such as innate immune response. In some embodiments, the method does not induce interferon and/or interleukin expression in the host cell. In some embodiments, the method does not induce IFN-β and/or IL-6 expression in the host cell.

The nucleic acids, including dRNAs, constructs thereof, and nucleic acids encoding ADAR may be delivered using any known methods in the art, including viral delivery or non-viral delivery.

Methods of non-viral delivery of nucleic acids include lipofection, nucleofection, microinjection, biolistics, virosomes, liposomes, immunoliposomes, polycation or lipid: nucleic acid conjugates, electroporation, nanoparticles, exosomes, microvesicles, or gene-gun, naked DNA and artificial virions.

The use of RNA or DNA viral based systems for the delivery of nucleic acids has high efficiency in targeting a virus to specific cells and trafficking the viral payload to the cellular nuclei. In certain embodiments, the method comprises introducing a viral vector (such as an AAV, e.g., scAAV, or a lentiviral vector) encoding the dRNA into the host cell. For example, the construct described herein may be any one of the viral vectors described in section III “dRNAs, constructs and libraries” below.

In some embodiments, the method comprises introducing a plasmid encoding the dRNA into the host cell. In some embodiments, the method comprises electroporation of the dRNA (e.g., synthetic dRNA) into the host cell. In some embodiments, the method comprises transfection of the dRNA into the host cell.

In certain embodiments, the efficiency of editing of the target RNA is at least about 10%, such as at least about any one of 15%, 20%, 25%, 30%, 35%, 40%, 45%, 50%, 55%, 60%, 65%, 70%, 75%, 80%, 85%, 90%, or higher. In some embodiments, the efficiency of editing of the target RNA is at least about 40%. In some embodiments, the efficiency of editing is determined by Sanger sequencing. In some embodiments, the efficiency of editing is determined by next-generation sequencing. In some embodiments, the efficiency of editing is determined by assessing expression of a reporter gene, such as a fluorescence reporter, e.g., EGFP.

In certain embodiments, the method has low off-target editing rate. In some embodiments, the method has lower than about 1% (e.g., no more than about any one of 0.5%, 0.1%, 0.05%, 0.01%, 0.001% or lower) editing efficiency on non-target As in the target RNA. In some embodiments, the method does not edit non-target As in the target RNA. In some embodiments, the method has lower than about 0.1% (e.g., no more than about any one of 0.05%, 0.01%, 0.005%, 0.001%, 0.0001% or lower) editing efficiency on As in non-target RNA.

After deamination, modification of the target RNA and/or the protein encoded by the target RNA, can be determined using different methods depending on the positions of the targeted adenosines in the target RNA. For example, in order to determine whether “A” has been edited to “I” in the target RNA, RNA sequencing methods known in the art can be used to detect the modification of the RNA sequence. When the target adenosine is located in the coding region of an mRNA, the RNA editing may cause changes to the amino acid sequence encoded by the mRNA. For example, point mutations may be introduced to the mRNA of an innate or acquired point mutation in the mRNA may be reversed to yield wild-type gene product(s) because of the conversion of “A” to “I”. Amino acid sequencing by methods known in the art can be used to find any changes of amino acid residues in the encoded protein. Modifications of a stop codon may be determined by assessing the presence of a functional, elongated, truncated, full-length and/or wild-type protein. For example, when the target adenosine is located in a UGA, UAG, or UAA stop codon, modification of the target adenosine residue (UGA or UAG) or As (UAA) may create a read-through mutation and/or an elongated protein, or a truncated protein encoded by the target RNA may be reversed to create a functional, full-length and/or wild-type protein. Editing of a target RNA may also generate an aberrant splice site, and/or alternative splice site in the target RNA, thus leading to an elongated, truncated, or misfolded protein, or an aberrant splicing or alternative splicing site encoded in the target RNA may be reversed to create a functional, correctly-folding, full-length and/or wild-type protein. In some embodiments, the present application contemplates editing of both innate and acquired genetic changes, for example, missense mutation, early stop codon, aberrant splicing or alternative splicing site encoded by a target RNA. Using known methods to assess the function of the protein encoded by the target RNA can find out whether the RNA editing achieves the desired effects. Because deamination of the adenosine (A) to an inosine (I) may correct a mutated A at the target position in a mutant RNA encoding a protein, identification of the deamination into inosine may provide assessment on whether a functional protein is present, or whether a disease or drug resistance-associated RNA caused by the presence of a mutated adenosine is reversed or partly reversed. Similarly, because deamination of the adenosine (A) to an inosine (I) may introduce a point mutation in the resulting protein, identification of the deamination into inosine may provide a functional indication for identifying a cause of disease or a relevant factor of a disease.

When the presence of a target adenosine causes aberrant splicing, the read-out may be the assessment of occurrence and frequency of aberrant splicing. On the other hand, when the deamination of a target adenosine is desirable to introduce a splice site, then similar approaches can be used to check whether the required type of splicing occurs. An exemplary suitable method to identify the presence of an inosine after deamination of the target adenosine is RT-PCR and sequencing, using methods that are well-known to the person skilled in the art.

The effects of deamination of target adenosine(s) include, for example, point mutation, early stop codon, aberrant splice site, alternative splice site and misfolding of the resulting protein. These effects may induce structural and functional changes of RNAs and/or proteins associated with diseases, whether they are genetically inherited or caused by acquired genetic mutations, or may induce structural and functional changes of RNAs and/or proteins associated with occurrence of drug resistance. Hence, the dRNAs, the constructs encoding the dRNAs, and the RNA editing methods of present application can be used in prevention or treatment of hereditary genetic diseases or conditions, or diseases or conditions associated with acquired genetic mutations by changing the structure and/or function of the disease-associated RNAs and/or proteins.

In some embodiments, the target RNA is a pre-messenger RNA. In some embodiments, the target RNA is a messenger RNA. In some embodiments, the target RNA is a regulatory RNA. In some embodiments, the target RNA a ribosomal RNA, a transfer RNA, a long non-coding RNA or a small RNA (e.g., miRNA, pri-miRNA, pre-miRNA, piRNA, siRNA, snoRNA, snRNA, exRNA or scaRNA). The effects of deamination of the target adenosines include, for example, structural and functional changes of the ribosomal RNA, transfer RNA, long non-coding RNA or small RNA (e.g., miRNA), including changes of three-dimensional structure and/or loss of function or gain of function of the target RNA. In some embodiments, deamination of the target As in the target RNA changes the expression level of one or more downstream molecules (e.g., protein, RNA and/or metabolites) of the target RNA. Changes of the expression level of the downstream molecules can be increase or decrease in the expression level.

Some embodiments of the present application involve multiplex editing of target RNAs in host cells, which are useful for screening different variants of a target gene or different genes in the host cells. In some embodiments, wherein the method comprises introducing a plurality of dRNAs to the host cells, at least two of the dRNAs of the plurality of dRNAs have different sequences and/or have different target RNAs. In some embodiments, each dRNA has a different sequence and/or different target RNA. In some embodiments, the method generates a plurality (e.g., at least 2, 3, 5, 10, 50, 100, 1000 or more) of modifications in a single target RNA in the host cells. In some embodiments, the method generates a modification in a plurality (e.g., at least 2, 3, 5, 10, 50, 100, 1000 or more) of target RNAs in the host cells. In some embodiments, the method comprises editing a plurality of target RNAs in a plurality of populations of host cells. In some embodiments, each population of host cells receive a different dRNA or a dRNAs having a different target RNA from the other populations of host cells.

Also provided are edited RNA or host cells having an edited RNA produced by any one of the methods described herein. In some embodiments, the edited RNA comprises an inosine. In some embodiments, the host cell comprises a target RNA having a missense mutation, an early stop codon, an alternative splice site, or an aberrant splice site. In some embodiments, the host cell comprises a mutant, truncated, or misfolded protein. In some embodiments, the method restores the function of the target RNA.

### III. dRNAs, Constructs and Libraries

The present application further provides dRNAs, constructs encoding dRNAs and libraries comprising a plurality of dRNAs or constructs thereof, which can be used in any one of the methods of RNA editing or methods of treatment described herein. It is intended that any of the features and parameters described herein for dRNAs or constructs can be combined with each other, as if each and every combination is individually described.

In one aspect, the present application provides a dRNA for editing a target RNA comprising a targeting RNA sequence that is capable of hybridizing to the target RNA to form a duplex RNA, wherein the duplex RNA comprises a bulge comprising a non-target adenosine in the target RNA. In some embodiments, the duplex RNA comprises a bulge at each non-target adenosine in the target RNA. In some embodiments, the targeting RNA sequence is complementary to the target RNA except for lacking one or more nucleotides opposite non-target adenosines in the target RNA. In some embodiments, the targeting RNA sequence has deletion of one or more uridine residues opposite one or more non-target adenosines in a sequence complementary to the target RNA. In some embodiments, the dRNA is a linear RNA. In some embodiments, the dRNA is a circular RNA. In some embodiments, the dRNA is a linear RNA capable of forming a circular RNA.

In one aspect, the present application provides a dRNA for editing a target RNA comprising a targeting RNA sequence that is capable of hybridizing to the target RNA, wherein the dRNA comprises a linker nucleic acid sequence flanking an end of the targeting RNA sequence, wherein the linker nucleic acid sequence does not substantially form any secondary structure with any part of the dRNA, and wherein the dRNA is a circular RNA or a linear RNA capable of forming a circular RNA. In some embodiments, the linker nucleic acid sequence is about 5 nt to about 500 nt long, such as about 50 nt to 200 nt long. In some embodiments, the linker nucleic acid sequence comprises a polyadenosine (polyA), polyguanosine (polyG), or polycytosine (polyC) sequence. In some embodiments, at least 50% of the linker nucleic acid sequence comprises adenosine. In some embodiments, the linker nucleic acid sequence comprises a dinucleotide repeat sequence, such as (AT)_{n,} wherein n is an integer greater or equal to 3. In some embodiments, the linker nucleic acid sequence comprises SEQ ID NO: 22. In some embodiments, the dRNA is a circular RNA. In some embodiments, the dRNA comprises a first linker nucleic acid sequence flanking the 5′ end of the targeting RNA sequence and a second linker nucleic acid sequence flanking the 3′ end of the targeting RNA sequence. In some embodiments, the dRNA comprises a first linker nucleic acid sequence flanking the 5′ end of the targeting RNA sequence and a second linker nucleic acid sequence replacing the 3′ end of the targeting RNA sequence. In some embodiments, the dRNA comprises a first linker nucleic acid sequence replacing the 5′ end of the targeting RNA sequence and a second linker nucleic acid sequence flanking the 3′ end of the targeting RNA sequence. In some embodiments, the dRNA comprises a first linker nucleic acid sequence replacing the 5′ end of the targeting RNA sequence and a second linker nucleic acid sequence replacing the 3′ end of the targeting RNA sequence.

In one aspect, the present application provides a dRNA for editing a target RNA comprising a targeting RNA sequence that is capable of hybridizing to the target RNA, wherein the duplex RNA comprises a bulge comprising a non-target adenosine in the target RNA, wherein the dRNA comprises a linker nucleic acid sequence flanking an end of the targeting RNA sequence, wherein the linker nucleic acid sequence does not substantially form any secondary structure with any part of the dRNA, and wherein the dRNA is a circular RNA or a linear RNA capable of forming a circular RNA. In some embodiments, the dRNA is a circular RNA. In some embodiments, the targeting RNA sequence is complementary to the target RNA except for lacking one or more nucleotides opposite non-target adenosines in the target RNA. In some embodiments, the linker nucleic acid sequence is about 5 nt to about 500 nt long, such as about 50 nt to 200 nt long. In some embodiments, the linker nucleic acid sequence comprises a polyadenosine (polyA), polyguanosine (polyG), or polycytosine (polyC) sequence. In some embodiments, the linker nucleic acid sequence comprises a dinucleotide repeat sequence, such as (AT)_{n,} wherein n is an integer greater or equal to 3. In some embodiments, the linker nucleic acid sequence comprises SEQ ID NO: 22. In some embodiments, the dRNA comprises a first linker nucleic acid sequence flanking the 5′ end of the targeting RNA sequence and a second linker nucleic acid sequence flanking the 3′ end of the targeting RNA sequence. In some embodiments, the dRNA comprises a first linker nucleic acid sequence flanking the 5′ end of the targeting RNA sequence and a second linker nucleic acid sequence replacing the 3′ end of the targeting RNA sequence. In some embodiments, the dRNA comprises a first linker nucleic acid sequence replacing the 5′ end of the targeting RNA sequence and a second linker nucleic acid sequence flanking the 3′ end of the targeting RNA sequence. In some embodiments, the dRNA comprises a first linker nucleic acid sequence replacing the 5′ end of the targeting RNA sequence and a second linker nucleic acid sequence replacing the 3′ end of the targeting RNA sequence.

In one aspect, the present application provides a construct comprising a nucleic acid sequence encoding any one of the dRNAs described herein. In certain embodiments, the construct is a viral vector or a plasmid. In some embodiments, the construct is an adeno-associated viral (AAV) vector. In some embodiments, the construct is a self-complementary AAV (scAAV) vector. In some embodiments, the construct encodes a single dRNA. In some embodiments, the construct encodes a plurality (e.g., about any one of 1, 2, 3, 4, 5, 10, 20 or more) dRNAs.

In one aspect, the present application provides a library comprising a plurality of the dRNAs or a plurality of the constructs described herein.

In one aspect, the present application provides a composition or a host cell comprising the deaminase-recruiting RNA or the construct described herein. In certain embodiments, the host cell is a prokaryotic cell or a eukaryotic cell. In some embodiments, the host cell is a mammalian cell. In some embodiments, the host cell is a human cell.

dRNAs

The dRNA of the present application comprises a targeting RNA sequence that hybridizes to the target RNA. The targeting RNA sequence is perfectly complementary or substantially complementarity to the target RNA to allow hybridization of the targeting RNA sequence to the target RNA. In some embodiments, the targeting RNA sequence has 100% sequence complementarity as the target RNA. In some embodiments, the targeting RNA sequence is at least about any one of 70%, 80%, 85%, 90%, 95%, 96%, 97%, 98%, or 99% or more complementary to over a continuous stretch of at least about any one of 20, 40, 60, 80, 100, 150, 200, or more nucleotides in the target RNA. In some embodiments, the dsRNA formed by hybridization between the targeting RNA sequence and the target RNA has one or more (e.g., 1, 2, 3, 4, 5, 6, 7, 8, 9, 10 or more) non-Watson-Crick base pairs (i.e., mismatches).

In some embodiments, the dsRNA (also referred herein as “duplex RNA”) formed by hybridization between the targeting RNA sequence and the target RNA has one or more unpaired (e.g., 1, 2, 3, 4, 5, 6, 7, 8, 9, 10 or more) nucleotides. In some embodiments, the dsRNA formed hybridization between the targeting RNA sequence and the target RNA has one or more non-target adenosines in the target RNA that are unpaired. In some embodiments, the dRNA lacks one or more nucleotides opposite one or more non-target adenosines in the target RNA. In some embodiments, the targeting RNA sequence in the dRNA lacks the nucleotide opposite each non-target adenosine in the target RNA. In some embodiments, the targeting RNA sequence in the dRNA has a deletion of two or more (e.g., 2, 3, 4, or more) consecutive nucleotides opposite a region comprising a non-target adenosine in the target RNA. In some embodiments, the targeting RNA sequence in the dRNA is complementary to the target RNA except for lacking one or more nucleotides opposite one or more non-target adenosines in the target RNA. In some embodiments, the targeting RNA sequence in the dRNA is complementary to the target RNA except lacking a nucleotide opposite each non-target adenosine in the target RNA.

Unpaired nucleotides in a dsRNA give rise to a bulge. In some embodiments, the target RNA hybridizes with the dRNA to form a dsRNA comprising a bulge comprising a non-target adenosine in the target RNA. The bulge in the dsRNA formed by hybridization of the dRNA with the target RNA comprises a non-target adenosine in the target RNA. The bulge maybe single nucleotide bulge, i.e., containing an unpaired non-target adenosine, or multi-nucleotide bulge, i.e., containing additional unpaired or mismatched nucleotides that flank the unpaired non-target adenosine. In some embodiments, the bulge may contain more than one (e.g., 2, 3, 4, 5 or more) unpaired nucleotides in the target RNA, i.e., the bulge is made of unpaired nucleotides that directly flanking the 5′ and/or the 3′ side of the non-target adenosine residue. In some embodiments, the bulge may contain one or more (e.g., 2, 3, 4, 5 or more) mismatched nucleotides directly flanking the 5′ and/or the 3′ side of the non-target adenosine residue. In some embodiments, the bulge comprises an unpaired non-target adenosine, one or more unpaired nucleotides flanking the 5′ and/or the 3′ side of the non-target adenosine residue, and one or more mismatched nucleotides flanking the 5′ and/or the 3′ side of the non-target adenosine residue. In some embodiments, the bulge is 1 nt, 2 nt, 3 nt, or longer.

In some embodiments, the duplex RNA comprises two or more bulges, such as any one of 2, 3, 4, 5, 6, or more bulges, wherein each bulge comprises a non-target adenosine in the target RNA. In some embodiments, the duplex RNA comprises a bulge at each non-target adenosine in the target RNA.

In some embodiments, the dsRNA formed by hybridization between the dRNA and the target RNA does not comprise a mismatch. In some embodiments, the dsRNA formed by hybridization between the dRNA and the target RNA comprises one or more, such as any one of 1, 2, 3, 4, 5, 6, 7 or more mismatches (e.g., the same type of different types of mismatches). In some embodiments, the dsRNA formed by hybridization between the dRNA and the target RNA comprises one or more kinds of mismatches, for example, 1, 2, 3, 4, 5, 6, 7 kinds of mismatches selected from the group consisting of G-A, C-A, U-C, A-A, G-G, C-C and U-U.

In some embodiments, the mismatch is upstream (5′) or downstream (3′) of the target adenosine, which may promote the efficiency of editing of the target adenosine at the target RNA. In some embodiments, the duplex RNA has additional mismatches upstream and/or downstream of the target adenosine.

In some embodiments, the targeting RNA sequence further comprises one or more guanosine(s), such as 1, 2, 3, 4, 5, 6, or more Gs, that is each directly opposite a non-target adenosine in the target RNA. In some embodiments, the targeting RNA sequence comprises two or more consecutive mismatch nucleotides (e.g., 2, 3, 4, 5, or more mismatch nucleotides) opposite a non-target adenosine in the target RNA.

In some embodiments, the dRNA comprises a targeting RNA sequence comprising a G opposite one or more non-target adenosines in the target RNA, and lacks a nucleotide opposite one or more non-target adenosines in the target RNA. The duplex RNA may have one or more bulges comprising one or more non-target adenosines, and at the dRNA comprises Gs opposite the other non-target adenosines in the target RNA.

In some embodiments, the target RNA comprises no more than about 20 non-target As, such as no more than about any one of 15, 10, 9, 8, 7, 6, 5, 4, 3, 2, or 1 non-target A. The U deletions and/or Gs opposite non-target As together with flanking mismatch or unpaired nucleotides in the dRNA may reduce off-target editing effects by ADAR.

In some embodiments, the dRNA comprises one or more linker nucleic acid sequences (also referred herein as “flanking linker sequence”) flanking an end of the targeting RNA sequence, wherein the linker nucleic acid sequence does not substantially form any secondary structure with any part of the dRNA. The inventors of the present application discovered that including of a flanking linker sequence may increase editing efficiency of a target adenosine in a target RNA. Without being bound by any theory, it is hypothesized that the linker nucleic acid sequence may increase flexibility of a circular dRNA, which may promote hybridization of the targeting RNA sequence to the target RNA.

In some embodiments, the dRNA comprises a single linker nucleic acid sequence. In some embodiments, the dRNA comprises a linker nucleic acid sequence at the 5′ end of the targeting RNA sequence. In some embodiments, the dRNA comprises a linker nucleic acid sequence at the 3′ end of the targeting RNA sequence. In some embodiments, the dRNA comprises a first linker nucleic acid sequence at the 5′ end of the targeting RNA sequence, and a second linker nucleic acid sequence at the 3′ end of the targeting RNA sequence. In some embodiments, the dRNA is a circular RNA comprising a linker nucleic acid sequence connecting directly or indirectly the 5′ end and the 3′ end of the targeting RNA sequence. The first linker nucleic acid sequence and the second linker nucleic acid sequence may have the same or different sequences.

In some embodiments, the linker nucleic acid sequence (including the first linker nucleic acid sequence and the second linker nucleic acid sequence) is at least about any one of 5, 10, 20, 30, 40, 50, 60, 70, 80, 90, 100, 150, 200, 250, 300, 350, 400, 450, or 500 nt long. In some embodiments, the linker nucleic acid sequence (including the first linker nucleic acid sequence and the second linker nucleic acid sequence) is no more than about any one of 500, 450, 400, 350, 300, 250, 200, 150, 100, 90, 80, 70, 60, 50, 40, 30, 20, 10, or 5 nt long. In some embodiments, the linker nucleic acid sequence (including the first linker nucleic acid sequence and the second linker nucleic acid sequence) is about any one of 5-10, 10-20, 20-50, 5-50, 10-100, 5-50, 50-100, 100-200, 200-300, 300-400, 400-500, 5-100, 5-200, 5-300, 5-400, 5-500, 50-200, 50-300, 50-400 or 50-500 nt long. In some embodiments, the linker nucleic acid sequence (including the first linker nucleic acid sequence and the second linker nucleic acid sequence) is about 50 nt long. In some embodiments, the first linker nucleic acid sequence and the second linker nucleic acid sequence have the same length. In some embodiments, the first linker nucleic acid sequence and the second linker nucleic acid sequence have different lengths.

The linker nucleic acid sequence (including the first linker nucleic acid sequence and the second linker nucleic acid sequence) does not substantially form any secondary structure with any part of the dRNA. Computational tools are known in the art to predict the secondary structure of RNAs, including, for example, RNAfold. In some embodiments, the linker nucleic acid sequence does not form a duplex region with a portion of the targeting RNA sequence that is more than about any one of 3, 4, 5, 6, or more basepairs long. In some embodiments, the linker nucleic acid sequence does not contain complementary regions having more than 3, 4, 5, or 6 nucleotides long. In some embodiments, the first linker nucleic acid sequence does not have a complementary region having more than 3, 4, 5, or 6 nucleotides long with respect to the second linker nucleic acid sequence.

The linker nucleic acid sequence (including the first linker nucleic acid sequence and the second linker nucleic acid sequence) could be a mononucleotide or dinucleotide repeat sequence, or a random sequence. In some embodiments, the linker nucleic acid sequence comprises a polyadenosine (polyA), polyguanosine (polyG), or polycytosine (polyC) sequence. In some embodiments, at least 50% of the linker nucleic acid sequence comprises adenosine. In some embodiments, the linker nucleic acid sequence comprises a dinucleotide repeat sequence, such as an AT or TA repeat sequence. In some embodiments, the linker nucleic acid sequence comprises (AT)_{n,} wherein n is an integer greater or equal to 3. In some embodiments, the linker nucleic acid sequence comprises SEQ ID NO: 22.

In some embodiments, the linker nucleic acid sequence serves as a ligation sequence that connects the 5′ end and the 3′ end of the targeting RNA sequence in a circular dRNA.

ADAR, for example, human ADAR enzymes edit double stranded RNA (dsRNA) structures with varying specificity, depending on a number of factors. One important factor is the degree of complementarity of the two strands making up the dsRNA sequence. Perfect complementarity of between the dRNA and the target RNA usually causes the catalytic domain of ADAR to deaminate adenosines in a non-discriminative manner. The specificity and efficiency of ADAR can be modified by introducing mismatches in the dsRNA region. For example, A-C mismatch is preferably recommended to increase the specificity and efficiency of deamination of the adenosine to be edited. Perfect complementarity is not necessarily required for a dsRNA formation between the dRNA and its target RNA, provided there is substantial complementarity for hybridization and formation of the dsRNA between the dRNA and the target RNA. In some embodiments, the dRNA sequence or single-stranded RNA region thereof has at least about any one of 70%, 80%, 85%, 90%, 95%, 96%, 97%, 98%, or 99% of sequence complementarity to the target RNA, when optimally aligned. Optimal alignment may be determined with the use of any suitable algorithm for aligning sequences, non-limiting examples of which include the Smith-Waterman algorithm, the Needleman-Wimsch algorithm, algorithms based on the Burrows-Wheeler Transform (e.g., the Burrows Wheeler Aligner).

The nucleotides neighboring the target adenosine also affect the specificity and efficiency of deamination. For example, the 5′ nearest neighbor of the target adenosine to be edited in the target RNA sequence has the preference U>C≈A>G and the 3′ nearest neighbor of the target adenosine to be edited in the target RNA sequence has the preference G>C>A≈U in terms of specificity and efficiency of deamination of adenosine. In some embodiments, when the target adenosine may be in a three-base motif selected from the group consisting of UAG, UAC, UAA, UAU, CAG, CAC, CAA, CAU, AAG, AAC, AAA, AAU, GAG, GAC, GAA and GAU in the target RNA, the specificity and efficiency of deamination of adenosine are higher than adenosines in other three-base motifs. In some embodiments, where the target adenosine to be edited is in the three-base motif UAG, UAC, UAA, UAU, CAG, CAC, AAG, AAC or AAA, the efficiency of deamination of adenosine is much higher than adenosines in other motifs. With respect to the same three-base motif, different designs of dRNA may also lead to different deamination efficiency. Taking the three-base motif UAG as an example, in some embodiments, when the dRNA comprises cytidine (C) directly opposite the target adenosine to be edited, adenosine (A) directly opposite the uridine, and cytidine (C), guanosine (G) or uridine (U) directly opposite the guanosine, the efficiency of deamination of the target adenosine is higher than that using other dRNA sequences. In some embodiments, when the dRNA comprises ACC, ACG or ACU opposite UAG of the target RNA, the editing efficiency of the A in the UAG of the target RNA may reach about 25%-90% (e.g., about 25%-80%, 25%-70%, 25%-60%, 25%-50%, 25%-40%, or 25%-30%).

Besides the target adenosines, there may be one or more adenosines in the target RNA (referred herein as “non-target A”), which are not desirable to be edited. With respect to these adenosines, it is preferable to reduce their editing efficiency as much as possible. The inventors of the present application discovered that deletion of a U opposite a non-target A, which results in formation of a bulge having an unpaired non-target A in the dRNA-target RNA duplex, significantly reduces off-target editing at the non-target A. The dRNA may further contain one or more unpaired nucleotides and/or one or more mismatched nucleotides that directly flank the 5′ or 3′ side of a non-target adenosine. As used herein, the term “unpaired” refers to a nucleotide in a first strand of a duplex nucleic acid that does not basepair with any nucleotide in a second strand of the duplex nucleic acid. In some embodiments, where guanosine is directly opposite an adenosine in the target RNA, the deamination efficiency is significantly decreased. Therefore, in order to decrease off-target deamination, dRNAs can be designed to have deletion of one or more nucleotides (e.g., U) opposite a first non-target adenosine, and/or to have a guanosine directly opposite a second non-target adenosine to be edited in the target RNA.

The desired level of specificity and efficiency of editing the target RNA sequence may depend on different applications. Following the instructions in the present patent application, those of skill in the art will be capable of designing a dRNA having complementary or substantially complementary sequence to the target RNA sequence according to their needs, and, with some trial and error, obtain their desired results. As used herein, the term “mismatch” refers to opposing nucleotides in a double stranded RNA (dsRNA) which do not form perfect base pairs according to the Watson-Crick base pairing rules. Mismatch base pairs include, for example, G-A, C-A, U-C, A-A, G-G, C-C, U-U base pairs. Taking A-C match as an example, where a target adenosine residue is to be edited in the target RNA, a dRNA is designed to comprise a C opposite the A to be edited, generating an A-C mismatch in the dsRNA formed by hybridization between the target RNA and dRNA.

The targeting RNA sequence in the dRNA is single-stranded. The dRNA may be entirely single-stranded or have one or more (e.g., 1, 2, 3, or more) double-stranded regions and/or one or more stem loop regions.

The dRNAs described herein comprise a targeting RNA sequence that is at least partially complementary to the target RNA. In certain embodiments, the targeting RNA sequence in the dRNA comprises at least about any one of 40, 45, 50, 55, 60, 65, 70, 75, 80, 90, 100, 110, 120, 130, 140, 150, 160, 170, 180, 190, 200, 210, 220, 230, 240, or 250 nucleotides (nt) long. In certain embodiments, the targeting RNA sequence in the dRNA comprises no more than about any one of 40, 45, 50, 55, 60, 65, 70, 75, 80, 90, 100, 110, 120, 130, 140, 150, 160, 170, 180, 190, 200, 210, 220, 230, 240, or 250 nucleotides. In certain embodiments, the targeting RNA sequence in the dRNA is about any one of 40-260, 45-250, 50-240, 60-230, 65-220, 70-220, 70-210, 70-200, 70-190, 70-180, 70-170, 70-160, 70-150, 70-140, 70-130, 70-120, 70-110, 70-100, 70-90, 70-80, 75-200, 80-190, 85-180, 90-170, 95-160, 100-200, 100-150, 100-175, 110-200, 110-160, 110-175, 110-150, 140-160, 105-140, or 105-155 nucleotides in length. In some embodiments, the targeting RNA sequence in the dRNA is about 100 to about 200 nt long. In some embodiments, the targeting RNA sequence in the dRNA is about 70 nt (e.g., 71 nt) long. In some embodiments, the targeting RNA sequence in the dRNA is about 120 nt (e.g., 121 nt) long. In some embodiments, the targeting RNA sequence in the dRNA is about 150 nt (e.g., 151 nt) long. In some embodiments, the targeting RNA sequence in the dRNA is about 170 nt (e.g., 171 nt) long. In some embodiments, the targeting RNA sequence in the dRNA is about 200 nt (e.g., 201 nt) long. In some embodiments, the targeting RNA sequence in the dRNA is about 220 nt (e.g., 221 nt) long.

In some embodiments, the targeting RNA sequence comprises a cytidine, adenosine or uridine directly opposite the target adenosine residue in the target RNA. In some embodiments, the targeting RNA sequence comprises a cytidine mismatch directly opposite the target adenosine residue in the target RNA. In some embodiments, the cytidine mismatch is located at least 5 nucleotides, e.g., at least 10, 15, 20, 25, 30, or more nucleotides, away from the 5′ end of the targeting RNA sequence. In some embodiments, the cytidine mismatch is located at least 20 nucleotides, e.g., at least 25, 30, 35, or more nucleotides, away from the 3′ end of the complementary RNA sequence. In some embodiments, the cytidine mismatch is not located within 20 (e.g., 15, 10, 5 or fewer) nucleotides away from the 3′ end of the targeting RNA sequence. In some embodiments, the cytidine mismatch is located at least 20 nucleotides (e.g., at least 25, 30, 35, or more nucleotides) away from the 3′ end and at least 5 nucleotides (e.g., at least 10, 15, 20, 25, 30, or more nucleotides) away from the 5′ end of the targeting RNA sequence. In some embodiments, the cytidine mismatch is located in the center of the targeting RNA sequence. In some embodiments, the cytidine mismatch is located within 20 nucleotides (e.g., 15, 10, 9, 8, 7, 6, 5, 4, 3, 2, or 1 nucleotide) of the center of the targeting sequence in the dRNA.

In certain embodiments, the 5′ nearest neighbor of the target adenosine residue is a nucleotide selected from U, C, A and G with the preference U>C≈A>G and the 3′ nearest neighbor of the target adenosine residue is a nucleotide selected from G, C, A and U with the preference G>C>A≈U. In some embodiments, the 5′ nearest neighbor of the target adenosine residue is U. In some embodiments, the 5′ nearest neighbor of the target adenosine residue is C or A. In some embodiments, the 3′ nearest neighbor of the target adenosine residue is G. In some embodiments, the 3′ nearest neighbor of the target adenosine residue is C.

In some embodiments, the target adenosine residue is in a three-base motif selected from the group consisting of UAG, UAC, UAA, UAU, CAG, CAC, CAA, CAU, AAG, AAC, AAA, AAU, GAG, GAC, GAA and GAU in the target RNA. In some embodiments, the three-base motif is UAG, and the dRNA comprises an A directly opposite the U in the three-base motif, a C directly opposite the target A, and a C, G or U directly opposite the G in the three-base motif. In certain embodiments, the three-base motif is UAG in the target RNA, and the dRNA comprises ACC, ACG or ACU that is opposite the UAG of the target RNA. In certain embodiments, the three-base motif is UAG in the target RNA, and the dRNA comprises ACC that is opposite the UAG of the target RNA.

In some embodiments, the dRNA, apart from the targeting RNA sequence, further comprises regions for stabilizing the dRNA, for example, one or more double-stranded regions and/or stem loop regions. In some embodiments, the double-stranded region or stem loop region of the dRNA comprises no more than about any one of 200, 150, 100, 50, 40, 30, 20, 10 or fewer base-pairs. In some embodiments, the dRNA does not comprise a stem loop or double-stranded region. In some embodiments, the dRNA comprises an ADAR-recruiting domain. In some embodiments, the dRNA does not comprise an ADAR-recruiting domain.

The dRNA may comprise one or more modifications. In some embodiments, the dRNA has one or more modified nucleotides, including nucleobase modification and/or backbone modification. Exemplary modifications to the RNA include, but are not limited to, phosphorothioate backbone modification, 2′-substitutions in the ribose (such as 2′-O-methyl and 2′-fluoro substitutions), LNA, and L-RNA.

In some embodiments, the dRNA does not comprise chemical modifications. In some embodiments, the dRNA does not comprise a chemically modified nucleotide, such as 2′-O-methyl nucleotide or a nucleotide having a phosphorothioate linkage. In some embodiments, the dRNA comprises 2′-O-methyl and phosphorothioate linkage modifications only at the first three and last three residues. In some embodiments, the dRNA is not an antisense oligonucleotide (ASO).

The dRNA may further comprise one or more additional expression elements that facilitate expression and/or circularization of the dRNA.

In some embodiments, the dRNA further comprises a 3′ exon sequence recognizable by a 3′ catalytic Group I intron fragment flanking the 5′ end of the targeting RNA sequence, and a 5′ exon sequence recognizable by a 5′ catalytic Group I intron fragment flanking the 3′ end of the targeting RNA sequence. In some embodiments, the Group I catalytic intron of the T4 phage Td gene is bisected in such a way to preserve structural elements critical for ribozyme folding. Exon fragment 2 is then ligated upstream of exon fragment 1, and a targeting RNA sequence (optionally with linker nucleic acid sequence(s) flanking the 5′ and/or the 3′ ends) is inserted between the exon-exon junction.

In some embodiments, the dRNA is a linear RNA that is capable of forming a circular RNA. In some embodiments, the circulation is performed using the Tornado expression system (“Twister-optimized RNA for durable overexpression”) as described in Litke, J. L. & Jaffrey, S. R. Highly efficient expression of circular RNA aptamers in cells using autocatalytic transcripts. Nat Biotechnol 37, 667-675 (2019), which is hereby incorporated herein by reference in its entirety. Briefly, Tornado-expressed transcripts contain an RNA of interest flanked by Twister ribozymes. A twister ribozyme is any catalytic RNA sequences that are capable of self-cleavage. The ribozymes rapidly undergo autocatalytic cleavage, leaving termini that are ligated by an RNA ligase.

In some embodiments, the dRNA comprises a targeting RNA sequence flanked (directly or indirectly) by a 5′ and/or 3′ ligation sequences. In some embodiments, the dRNA comprises a 3′ ligation sequence. In some embodiments, the dRNA comprises a 5′ ligation sequence. In some embodiments, the dRNA comprises a 3′ ligation sequence and a 5′ ligation sequence. In some embodiments, the 3′ ligation sequence and the 5′ ligation sequence are at least partially complementary to each other. In some embodiments, the 3′ ligation sequence and the 5′ ligation sequence are at least about 50%, at least about 55%, at least about 60%, at least about 65%, at least about 70%, at least about 75%, at least about 80%, at least about 85%, at least about 90%, at least about 95%, at least about 96%, at least about 97%, at least about 98% or at least about 99% complementary to each other. In some embodiments, the 3′ ligation sequence and the 5′ ligation sequence are fully complementary to each other. In some embodiments, the 5′ and/or 3′ ligation sequences are further flanked by the 5′-Twister ribozyme and/or 3′-Twister ribozymes, respectively.

In some embodiments, the dRNA is a linear RNA capable of forming a circular RNA, wherein the dRNA comprises, from the 5′ to the 3′: a 5′ ligation sequence, a first linker nucleic acid sequence, a targeting RNA sequence, a second linker nucleic acid sequence, and a 3′ ligation sequence. In some embodiments, the dRNA is a linear RNA capable of forming a circular RNA, wherein the dRNA comprises, from the 5′ to the 3′: a 5′ ligation sequence, a linker nucleic acid sequence, a targeting RNA sequence, and a 3′ ligation sequence. In some embodiments, the dRNA is a linear RNA capable of forming a circular RNA, wherein the dRNA comprises, from the 5′ to the 3′: a 5′ ligation sequence, a targeting RNA sequence, a linker nucleic acid sequence, and a 3′ ligation sequence. In some embodiments, the dRNA is a linear RNA capable of forming a circular RNA, wherein the dRNA comprises, from the 5′ to the 3′: a 5′ ligation sequence, a targeting RNA sequence, and a 3′ ligation sequence. In some embodiments, the 3′ ligation sequence comprises SEQ ID NO: 11. In some embodiments, the 5′ ligation sequence comprises SEQ ID NO: 12.

In some embodiments, the dRNA is a circular RNA comprising a ligation sequence connecting directly or indirectly the 5′ end and the 3′ end of the targeting RNA sequence. In some embodiments, the ligation sequence comprises a 5′ ligation sequence and a 3′ ligation sequence that are ligated to each other via a ligase, e.g., T4 RNA ligase such as Rnl1 or Rnl2. In some embodiments, the ligation sequence comprises SEQ ID NO: 10.

In some embodiments, the dRNA is a circular RNA comprising in a clockwise direction: a ligation sequence, a first linker nucleic acid sequence, the targeting RNA sequence, and a second linker nucleic acid sequence, wherein the ligation sequence directly connects the 5′ end of the first linker nucleic acid sequence to the 3′ end of the second linker nucleic acid sequence. In some embodiments, the dRNA is a circular RNA comprising in a clockwise direction: a ligation sequence, a linker nucleic acid sequence, the targeting RNA sequence, wherein the ligation sequence directly connects the 5′ end of the linker nucleic acid sequence to the 3′ end of the targeting RNA sequence. In some embodiments, the dRNA is a circular RNA comprising in a clockwise direction: a ligation sequence, the targeting RNA sequence, and a linker nucleic acid sequence, wherein the ligation sequence directly connects the 5′ end of the targeting RNA sequence to the 3′ end of the linker nucleic acid sequence. In some embodiments, the dRNA is a circular RNA comprising a ligation sequence and the targeting RNA sequence, wherein the ligation sequence directly connects the 5′ end of the targeting RNA sequence to the 3′ end of the targeting RNA sequence.

In some embodiments, the 3′ ligation sequence and the 5′ ligation sequence are independently at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides or at least about 100 nucleotides in length. In some embodiments, the 3′ ligation sequence and the 5′ ligation sequence are independently about 20-30 nucleotides, about 30-40 nucleotides, about 40-50 nucleotides, about 50-60 nucleotides, about 60-70 nucleotides, about 70-80 nucleotides, about 80-90 nucleotides, about 90-100 nucleotides, about 100-125 nucleotides, about 125-150 nucleotides, about 20-50 nucleotides, about 50-100 nucleotides or about 100-150 nucleotides in length.

In some embodiments, the dRNA is circularized by an RNA ligase. Non-limiting examples of RNA ligase include: RtcB, T4 RNA Ligase 1 (Rnl1), T4 RNA Ligase 2 (Rnl2), Rnl3 and Trl1. In some embodiments, the RNA ligase is expressed endogenously in the host cell. In some embodiments, the RNA ligase is RNA ligase RtcB. In some embodiments, the method further comprises introducing an RNA ligase (e.g., RtcB) into the host cell.

In some embodiments, the dRNA is circularized before being introduced to the host cell. In some embodiments, the dRNA is chemically synthesized. In some embodiments, the dRNA is circularized through in vitro enzymatic ligation (e.g., using RNA or DNA ligase) or chemical ligation (e.g., using cyanogen bromide or a similar condensing agent).

The dRNAs described herein do not comprise a tracrRNA, crRNA or gRNA used in a CRISPR/Cas system. In some embodiments, the dRNA does not comprise an ADAR-recruiting domain. “ADAR-recruiting domain” can be a nucleotide sequence or structure that binds at high affinity to ADAR, or a nucleotide sequence that binds to a binding partner fused to ADAR in an engineered ADAR construct. Exemplary ADAR-recruiting domains include, but are not limited to, GluR-2, GluR-B (R/G), GluR-B (Q/R), GluR-6 (R/G), 5HT2C, and FlnA (Q/R) domain; see, for example, Wahlstedt, Helene, and Marie, “Site-selective versus promiscuous A-to-I editing.” Wiley Interdisciplinary Reviews: RNA 2.6 (2011): 761-771, which is incorporated herein by reference in its entirety. In some embodiments, the dRNA does not comprise a double-stranded portion. In some embodiments, the dRNA does not comprise a hairpin, such as MS2 stem loop. In some embodiments, the dRNA is single stranded.

In some embodiments, the dRNA comprises a snoRNA sequence linked to the 5′ end of the targeting RNA sequence (“5′ snoRNA sequence”). In some embodiments, the dRNA comprises a snoRNA sequence linked to the 3′ end of the targeting RNA sequence (“3′ snoRNA sequence”). In some embodiments, the dRNA comprises a snoRNA sequence linked to the 5′ end of the targeting RNA sequence (“5′ snoRNA sequence”) and a snoRNA sequence linked to the 3′ end of the targeting RNA sequence (“3′ snoRNA sequence”). In some embodiments, the snoRNA sequence is at least about 50 nucleotides, at least about 60 nucleotides, at least about 70 nucleotides, at least about 80 nucleotides, at least about 90 nucleotides, at least about 100 nucleotides, at least about 110 nucleotides, at least about 120 nucleotides, at least about 130 nucleotides, at least about 140 nucleotides, at least about 150 nucleotides, at least about 160 nucleotides, at least about 170 nucleotides, at least about 180 nucleotides, at least about 190 nucleotides or at least about 200 nucleotides in length. In some embodiments, the snoRNA sequence is about 50-75 nucleotides, about 75-100 nucleotides, about 100-125 nucleotides, about 125-150 nucleotides, about 150-175 nucleotides, about 175-200 nucleotides, about 50-100 nucleotides, about 100-150 nucleotides, about 150-200 nucleotides, about 125-175 nucleotides, or about 100-200 nucleotides in length. In some embodiments, the snoRNA sequence is a C/D Box snoRNA sequence. In some embodiments, the snoRNA sequence is an H/ACA Box snoRNA sequence. In some embodiments, the snoRNA sequence is a composite C/D Box and H/ACA Box snoRNA sequence. In some embodiments, the snoRNA sequence is an orphan snoRNA sequence.

Small nucleolar RNAs (snoRNAs) are small non-coding RNA molecules that are known to guide chemical modifications of other RNAs such as ribosomal RNAs, transfer RNAs, and small nuclear RNAs. There are two major groups of snoRNAs according to their specific secondary structure features: box C/D and box H/ACA. Both structural features of snoRNAs enable them binding to corresponding RNA binding proteins (RBPs) along with accessory proteins, forming functional small nucleolar ribonucleoprotein (snoRNP) complexes. Box C/D snoRNAs are believed to be associated with methylation, while H/ACA box snoRNAs are believed to be associated with pseudouridylation. Other families of snoRNAs include, for example, composite H/ACA and C/D box snoRNA and orphan snoRNAs. The snoRNA sequence described herein can comprise a naturally-occurring snoRNA, a portion thereof, or a variant thereof.

### Constructs

The present application provides constructs encoding the dRNAs and/or ADAR. In some embodiments, there is provided a construct (e.g., vector, such as viral vector) comprising a nucleotide sequence encoding the dRNA. In some embodiments, there is provided a construct (e.g., vector, such as viral vector) comprising a nucleotide sequence encoding the ADAR. In some embodiments, there is provided a construct comprising a first nucleotide sequence encoding the dRNA and a second nucleotide sequence encoding the ADAR. In some embodiments, the first nucleotide sequence and the second nucleotide sequence are operably linked to the same promoter. In some embodiments, the first nucleotide sequence and the second nucleotide sequence are operably linked to different promoters. In some embodiments, the promoter is inducible. In some embodiments, the construct does not encode for the ADAR. In some embodiments, the vector further comprises nucleic acid sequence(s) encoding an inhibitor of ADAR3 (e.g., ADAR3 shRNA or siRNA) and/or a stimulator of interferon (e.g., IFN-α).

The term “construct” as used herein refers to DNA or RNA molecules that comprise a coding nucleic acid sequence that can be transcribed into RNAs or expressed into proteins. In some embodiments, the construct contains one or more regulatory elements operably linked to the nucleic acid sequence encoding the RNA or protein. When the construct is introduced into a host cell, under suitable conditions, the coding nucleic acid sequence in the construct can be transcribed or expressed.

The constructs described herein may comprise a promoter that is operably linked to the nucleic acid sequence encoding the dRNA, such that the promoter controls the transcription or expression of the coding nucleotide sequence. The promoter may be positioned 5′ (upstream) of a coding nucleotide sequence under its control. The distance between the promoter and the coding sequence may be approximately the same as the distance between that promoter and the gene it controls in the gene from which the promoter is derived. As is known in the art, variation in this distance may be accommodated without loss of promoter function. In some embodiments, the construct comprises a 5′ UTR and/or a 3′UTR that regulates the transcription or expression of the coding nucleotide sequence. In some embodiments, the promoter is driving expression of two or more dRNAs.

The promoter may be a polymerase II promoter (“Pol II promoter”) or a polymerase III promoter (“Pol III promoter”). In some embodiments, wherein the dRNA is a linear RNA, the construct comprises a Pol II promoter operably linked to a nucleic acid sequence encoding the dRNA. Non-limiting examples of Pol II promoters include: CMV, SV40, EF-1α, CAG and RSV. In some embodiments, the Pol II promoter is a CMV promoter. In some embodiments, the CMV promoter comprises the nucleic acid sequence of SEQ ID NO: 5.

In some embodiments, wherein the dRNA is a circular RNA or a linear RNA capable of forming a circular RNA, the construct comprises a Pol III promoter. In some embodiments, the promoter is a U6 promoter. In some embodiments, the U6 promoter comprises the nucleic acid sequence of SEQ ID NO: 6.

In some embodiments, the construct is a vector encoding any one of the dRNAs disclosed in the present application. The term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the transcription or expression of coding nucleotide sequences to which they are operatively linked. Such vectors are referred to herein as “expression vectors”.

In some embodiments, the construct is a viral vector. In some embodiments, the construct is lentivirus vector. In some embodiments, the vector is a recombinant adeno-associated virus (rAAV) vector. Use of any AAV serotype is considered within the scope of the present disclosure. In some embodiments, the rAAV vector is a vector derived from an AAV serotype, including without limitation, AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV capsid serotype or the like. In some embodiments, the construct is flanked by one or more AAV inverted terminal repeat (ITR) sequences. In some embodiments, the construct is flanked by two AAV ITRs. In some embodiments, the AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype ITRs. In some embodiments, the AAV ITRs are AAV2 ITRs.

In some embodiments, the vector further comprises a stuffer nucleic acid. In some embodiments, the stuffer nucleic acid is located upstream or downstream of the nucleic acid encoding the dRNA. In some embodiments, the vector is a self-complementary rAAV vector. In some embodiments, the vector comprises first nucleic acid sequence encoding the dRNA and a second nucleic acid sequence encoding a complement of the dRNA, wherein the first nucleic acid sequence can form intrastrand base pairs with the second nucleic acid sequence along most or all of its length. In some embodiments, the first nucleic acid sequence and the second nucleic acid sequence are linked by a mutated AAV ITR, wherein the mutated AAV ITR comprises a deletion of the D region and comprises a mutation of the terminal resolution sequence. In some embodiments, the vector is encapsidated in a rAAV particle. In some embodiments, the AAV viral particle comprises an AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV2/2-7m8, AAV DJ, AAV2 N587A, AAV2 E548A, AAV2 N708A, AAV2 V708K, AAV2-HBKO, AAVDJ8, AAVPHP.B, AAVPHP.eB, AAVBR1, AAVHSC15, AAVHSC17, goat AAV, AAV1/AAV2 chimeric, bovine AAV, mouse AAV, or rAAV2/HBoV1 serotype capsid.

In some embodiments, the construct further comprises a 3′ twister ribozyme sequence linked to the 3′ end of the nucleic acid encoding the dRNA. In some embodiments, the construct further comprises a 5′ twister ribozyme sequence linked to the 5′ end of the nucleic acid sequence encoding the dRNA. In some embodiments, the construct further comprises a 3′ twister ribozyme sequence linked to the 3′ end of the nucleic acid sequence encoding the dRNA and a 5′ twister ribozyme sequence linked to the 5′ end of the nucleic acid encoding the dRNA. In some embodiments, the 3′ twister sequence is twister P3 U2A and the 5′ twister sequence is twister P1. In some embodiments, wherein the 5′ twister sequence is twister P3 U2A and the 3′ twister sequence is twister P1. In some embodiments, the dRNA undergoes autocatalytic cleavage. In some embodiments, the catalyzed dRNA product comprises a 5′-hydroxyl group and a 2′,3′-cyclic phosphate at the 3′ terminus.

### Method of Preparation

The dRNAs described herein may be prepared using any known methods in the art, including chemical synthesis and in vitro transcription. Circular dRNAs may be prepared by chemical ligation, enzymatic ligation, or ribozyme autocatalysis of linear RNAs. In some embodiments, the circular dRNA is prepared by circularizing a linear RNA in vitro.

In some embodiments, the present application provides a linear RNA capable of forming the circular dRNA of any one of the embodiments described above. In some embodiments, the linear RNA can circularized by chemical circularization methods using cyanogen bromide or a similar condensing agent. In some embodiments, the linear RNA can be circularized by autocatalysis of a Group I intron comprising a 5′ catalytic Group I intron fragment and a 3′ catalytic Group I intron fragment. In some embodiments, the linear RNA can be circularized by a ligase. In some embodiments, the linear RNA can be circularized by a T4 RNA ligase. In some embodiments, the linear RNA can be circularized by a DNA ligase. Suitable ligases include, but are not limited to a T4 DNA ligase (T4 Dnl), a T4 RNA ligase 1 (T4 Rnl1) and a T4 RNA ligase 2 (T4 Rnl2). The circular dRNA may be purified using known methods in the art, for example, by gel-purification or by high-performance liquid chromatography (HPLC).

In some embodiments, a linear RNA can be circularized by chemical methods to provide a circular dRNA. In some chemical methods, the 5′-end and the 3′-end of the nucleic acid (e.g., a linear circular polyribonucleotide) includes chemically reactive groups that, when close together, may form a new covalent linkage between the 5′-end and the 3′-end of the molecule. The 5′-end may contain an NHS ester reactive group and the 3′-end may contain a 3′-amino terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In some embodiments, a circular dRNA can be obtained by circularizing a linear RNA by ribozyme autocatalysis. In some embodiments, the linear RNA is circularized in vitro. In some embodiments, circularization by ribozyme autocatalysis comprises (a) subjecting the linear RNA to a condition that activates autocatalysis of the Group I intron (or 5′ and 3′ catalytic Group I intron fragments thereof) to provide a circularized RNA product; and (b) isolating the circularized RNA product, thereby providing the circular dRNA.

In some embodiments, the method comprises a step of obtaining the linear RNA by first cloning the sequence encoding the linearized RNAs into a plasmid vector, and then linearizing the recombinant plasmids. In some embodiments, the recombinant plasmids are linearized by restriction enzyme digestion. In some embodiments, the recombinant plasmids are linearized by PCR amplification. In some embodiments, the method further comprises performing in vitro transcription with the linearized plasmid template. In some embodiments, the in vitro transcription is driven by a T7 promoter. In some embodiments, the method further comprises purifying the linear RNA transcripts. In some embodiments, the linear RNAs are purified by gel purification.

In some embodiments, the present application provides a method of cyclizing a linear RNA (e.g., purified linear RNA) by ribozyme autocatalysis of the Group I intron. During splicing, the 3′ hydroxyl group of a guanosine nucleotide engages in a transesterification reaction at the 5′ splice site. The 5′ intron half is excised, and the freed hydroxyl group at the end of the intermediate engages in a second transesterification at the 3′ splice site, resulting in circularization of the intervening region and excision of the 3′ intron. In some embodiments, the condition that activates autocatalysis of the Group I intron or 5′ and 3′ catalytic Group I intron fragments is the addition of GTPs and Mg²⁺. In some embodiments, there is provided a step of cyclizing the linear RNAs by adding GTPs and Mg²⁺ at 55° C. for 15 min. In some embodiments, the method further comprises treating with RNase R to digest the linear RNA transcripts. In some embodiments, the method further comprises isolating the circular dRNA. In some embodiments, the step of isolating the circular dRNA comprises gel-purifying the circular dRNA.

In some embodiments, a circular dRNA can be obtained by circularizing a linear RNA using a ligase such as a RNA ligase. In some embodiments, the linear RNA is circularized in vitro. In some embodiments, the linear RNA can be circularized by a T4 RNA ligase. In some embodiments, the linear RNA comprises a 5′ ligation sequence at the 5′ end of the targeting RNA sequence, and a 3′ ligation sequence at the 3′ end of the targeting RNA sequence, wherein the 5′ ligation sequence and the 3′ ligation sequence can be ligated to each other via the RNA ligase. In non-limiting examples, the linear RNA can be circularized by a ligase such as a T4 DNA ligase (T4 Dnl), T4 RNA ligase 1 (T4 Rnl1), and T4 RNA ligase 2 (T4 Rnl2). The linear RNA may be circularized with or without the presence of a single stranded nucleic acid adaptor, e.g., a splint DNA.

In some embodiments, a DNA or RNA ligase may be used to enzymatically link a 5′-phosphorylated nucleic acid molecule (e.g., a linear RNA) to the 3′-hydroxyl group of a nucleic acid (e.g., a linear nucleic acid) forming a new phosphodiester linkage. In an example reaction, a linear circular RNA is incubated at 37° C. for 1 hour with 1-10 units of T4 RNA ligase (New England Biolabs, Ipswich, Mass.) according to the manufacturer's protocol. The ligation reaction may occur in the presence of a linear nucleic acid capable of base-pairing with both the 5′- and 3′-region in juxtaposition to assist the enzymatic ligation reaction. In some embodiments, the ligation is splint ligation. For example, a splint ligase, like SPLINTR® ligase, can be used for splint ligation. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide. In some embodiments, a DNA or RNA ligase may be used in the synthesis of a circular dRNA. As a non-limiting example, the ligase may be a circ ligase or circular ligase.

### IV. Methods of Treatment

The RNA editing methods and compositions described herein may be used to treat or prevent a disease or condition in an individual, including, but not limited to hereditary genetic diseases and drug resistance.

In some embodiments, there is provided a method of editing a target RNA in a cell of an individual (e.g., human individual) ex vivo, comprising editing the target RNA using any one of the methods of RNA editing described herein.

In some embodiments, there is provided a method of treating or preventing a disease or condition in an individual (e.g., human individual), comprising editing a target RNA associated with the disease or condition in a cell of the individual using any one of the methods of RNA editing described herein, wherein the dRNA comprises a targeting RNA sequence that hybridizes to a target RNA associated with the disease or condition. In some embodiments, the method comprises introducing the dRNA or the construct comprising a nucleic acid encoding the dRNA into an isolated cell of the individual ex vivo. In some embodiments, the method comprises comprising administering an effective amount of the dRNA or the construct comprising a nucleic acid encoding the dRNA to the individual.

In some embodiments, the target RNA is associated with a disease or condition of the individual. In some embodiments, the disease or condition is a hereditary genetic disease, or a disease or condition associated with one or more acquired genetic mutations (e.g., drug resistance). In some embodiments, the method further comprises obtaining the cell from the individual. In some embodiments, the ADAR is an endogenously expressed ADAR in the isolated cell. In some embodiments, the method comprises introducing the ADAR or a construct comprising a nucleic acid encoding the ADAR to the isolated cell. In some embodiments, the method further comprises culturing the cell having the edited RNA. In some embodiments, the method further comprises administering the cell having the edited RNA to the individual. In some embodiments, the disease or condition is a hereditary genetic disease, or a disease or condition associated with one or more acquired genetic mutations (e.g., drug resistance).

Diseases and conditions suitable for treatment using the methods of the present application include diseases associated with a mutation, such as a G to A mutation, e.g., a G to A mutation that results in missense mutation, early stop codon, aberrant splicing, or alternative splicing in an RNA transcript. Examples of disease-associated mutations that may be restored by the methods of the present application include, but are not limited to, TP53^{W53X} (e.g., 158G>A) associated with cancer, IDUA^{W402X} (e.g., TGG>TAG mutation in exon 9) associated with Mucopolysaccharidosis type I (MPS I), COL3A1^{W1278X} (e.g., 3833G>A mutation) associated with Ehlers-Danlos syndrome, BMPR2^{W298X} (e.g., 893G>A) associated with primary pulmonary hypertension, AHI1^{W725X} (e.g., 2174G>A) associated with Joubert syndrome, FANCC^{W506X} (e.g., 1517G>A) associated with Fanconi anemia, MYBPC3^{W1098X} (e.g., 3293G>A) associated with primary familial hypertrophic cardiomyopathy, and IL2RG^{W237X} (e.g., 710G>A) associated with X-linked severe combined immunodeficiency. In some embodiments, the disease or condition is a cancer. In some embodiments, the disease or condition is a monogenetic disease. In some embodiments, the disease or condition is a polygenetic disease.

In some embodiments, there is provided a method of treating a cancer associated with a target RNA having a mutation (e.g., G>A mutation) in an individual, comprising editing the target RNA in a cell of the individual using any one of the methods of RNA editing described herein. In some embodiments, the target RNA is TP53^{W53X} (e.g., 158G>A).

In some embodiments, there is provided a method of treating MIPS I (e.g., Hurler syndrome or Scheie syndrome) associated with a target RNA having a mutation (e.g., G>A mutation) in an individual, comprising editing the target RNA in a cell of the individual using any one of the methods of RNA editing described herein. In some embodiments, the target RNA is IDUA^{W402X} (e.g., TGG>TAG mutation in exon 9).

In some embodiments, there is provided a method of treating a disease or condition Ehlers-Danlos syndrome associated with a target RNA having a mutation (e.g., G>A mutation) in an individual, comprising editing the target RNA in a cell of the individual using any one of the methods of RNA editing described herein. In some embodiments, the target RNA is COL3A1^{W1278X} (e.g., 3833G>A mutation).

In some embodiments, there is provided a method of treating primary pulmonary hypertension associated with a target RNA having a mutation (e.g., G>A mutation) in an individual, comprising editing the target RNA in a cell of the individual using any one of the methods of RNA editing described herein. In some embodiments, the target RNA is BMPR2^{W298X} (e.g., 893G>A).

In some embodiments, there is provided a method of treating Joubert syndrome associated with a target RNA having a mutation (e.g., G>A mutation) in an individual, comprising editing the target RNA in a cell of the individual using any one of the methods of RNA editing described herein. In some embodiments, the target RNA is AHI1^{W725X} (e.g., 2174G>A).

In some embodiments, there is provided a method of treating Fanconi anemia associated with a target RNA having a mutation (e.g., G>A mutation) in an individual, comprising editing the target RNA in a cell of the individual using any one of the methods of RNA editing described herein. In some embodiments, the target RNA is FANCC^{W506X} (e.g., 1517G>A).

In some embodiments, there is provided a method of treating primary familial hypertrophic cardiomyopathy associated with a target RNA having a mutation (e.g., G>A mutation) in an individual, comprising editing the target RNA in a cell of the individual using any one of the methods of RNA editing described herein. In some embodiments, the target RNA is MYBPC3^{W1098X} (e.g., 3293G>A).

In some embodiments, there is provided a method of treating X-linked severe combined immunodeficiency associated with a target RNA having a mutation (e.g., G>A mutation) in an individual, comprising editing the target RNA in a cell of the individual using any one of the methods of RNA editing described herein. In some embodiments, the target RNA is IL2RG^{W237X} (e.g., 710G>A).

In some embodiments, there is provided a method of treating hyperglycemia associated with a target RNA having a mutation (e.g., G>A mutation) in an individual, comprising editing the target RNA in a cell of the individual using any one of the methods of RNA editing described herein. In some embodiments, the target RNA is MALAT1.

In some embodiments, there is provided a method of treating Charcot-Marie-Tooth disease 2B (CMT2B) associated with a target RNA having a mutation (e.g., G>A mutation) in an individual, comprising editing the target RNA in a cell of the individual using any one of the methods of RNA editing described herein. In some embodiments, the target RNA is RAB7A.

Generally, dosages, schedules, and routes of administration of the compositions (e.g., dRNA or construct comprising a nucleic acid encoding dRNA) may be determined according to the size and condition of the individual, and according to standard pharmaceutical practice. Exemplary routes of administration include intravenous, intra-arterial, intraperitoneal, intrapulmonary, intravesicular, intramuscular, intra-tracheal, subcutaneous, intraocular, intrathecal, or transdermal.

The RNA editing methods of the present application not only can be used in animal cells, for example mammalian cells, but also may be used in modification of RNAs of plant or fungi, for example, in plants or fungi that have endogenously expressed ADARs. The methods described herein can be used to generate genetically engineered plant and fungi with improved properties.

Further provided are any one of the dRNAs, constructs, cells having edited RNA, and compositions described herein for use in any one of the methods of treatment described herein, and any one of the dRNAs, constructs, edited cells, and compositions described herein in the manufacture of a medicament for treating a disease or condition.

### V. Compositions, Kits, and Articles of Manufacture

Also provided herein are compositions (such as pharmaceutical compositions) comprising any one of the dRNAs, constructs, libraries, or host cells having edited RNA as described herein.

In some embodiments, there is provided a pharmaceutical composition comprising any one of the dRNAs or constructs encoding the dRNA described herein, and a pharmaceutically acceptable carriers, excipients or stabilizers (Remington's Pharmaceutical Sciences 16th edition, Osol, A. Ed. (1980)). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). In some embodiments, lyophilized formulations are provided. Pharmaceutical compositions to be used for in vivo administration must be sterile. This is readily accomplished by, e.g., filtration through sterile filtration membranes.

Further provided are kits useful for any one of the methods of RNA editing or methods of treatment described herein, comprising any one of the dRNAs, constructs, compositions, libraries, or edited host cells as described herein.

In some embodiments, there is provided a kit for editing a target RNA in a host cell, comprising a dRNA or a construct comprising a nucleic acid encoding the dRNA, wherein the dRNA comprises a targeting RNA sequence that hybridizes to a target RNA associated with the disease or condition to form a duplex RNA, wherein the duplex RNA comprises a bulge comprising a non-target adenosine in the target RNA, wherein the dRNA is capable of recruiting an ADAR to deaminate a target adenosine residue in the target RNA. In some embodiments, the dRNA is circular. In some embodiments, the dRNA comprises a linker nucleic acid sequence flanking an end of the targeting RNA sequence.

In some embodiments, there is provided a kit for editing a target RNA in a host cell, comprising a dRNA or a construct comprising a nucleic acid encoding the dRNA, wherein the dRNA comprises a targeting RNA sequence that hybridizes to a target RNA associated with the disease or condition, wherein the dRNA comprises a linker nucleic acid sequence flanking an end of the targeting RNA sequence, wherein the linker nucleic acid sequence does not substantially form any secondary structure with any part of the dRNA, wherein the dRNA is capable of recruiting an ADAR to deaminate a target adenosine residue in the target RNA, and wherein the dRNA is a circular RNA or a linear RNA capable of forming a circular RNA.

In some embodiments, the kit further comprises an ADAR or a construct comprising a nucleic acid encoding an ADAR. In some embodiments, the kit further comprises an inhibitor of ADAR3 or a construct thereof. In some embodiments, the kit further comprises a stimulator of interferon or a construct thereof. In some embodiments, the kit further comprises an instruction for carrying out any one of the RNA editing methods or methods of treatment described herein.

The kits of the present application are in suitable packaging. Suitable packaging includes, but is not limited to, vials, bottles, jars, flexible packaging (e.g., sealed Mylar or plastic bags), and the like. Kits may optionally provide additional components such as transfection or transduction reagents, cell culturing medium, buffers, and interpretative information.

The present application thus also provides articles of manufacture. The article of manufacture can comprise a container and a label or package insert on or associated with the container. Suitable containers include vials (such as sealed vials), bottles, jars, flexible packaging, and the like. In some embodiments, the container holds a pharmaceutical composition, and may have a sterile access port (for example, the container may be an intravenous solution bag or a vial having a stopper pierceable by a hypodermic injection needle). The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical compositions and instructions for use, packaged in quantities sufficient for storage and use in pharmacies, for example, hospital pharmacies and compounding pharmacies.

### EXAMPLES

The examples below are intended to be purely exemplary of the present application and should therefore not be considered to limit the invention in any way. The following exemplary embodiments and examples and detailed description are offered by way of illustration and not by way of limitation.

### Materials and Methods

### Plasmid Construction

For linear arRNA-expressing construct, the sequences of arRNAs were synthesized and golden-gate cloned into pLenti-sgRNA-lib 2.0 (Addgene no. 89638) backbone, and the transcription of arRNA was driven by hU6 or CMV promoter respectively. For genetic encoded circ-arRNA-expressing construct, we first constructed a cloning vector based on the pLenti-sgRNA-lib 2.0 vector that included the Twister P3 U2A, 5′ ligation sequence, 3′ ligation sequence and Twister P1⁴⁸. Then the sequences of arRNAs were synthesized and golden gate cloned into the autocatalytic circular RNA expression vector.

To increase editing efficiency further, circ-arRNA₁₅₁ were flanked by 20 nt spacer and 30 nt poly AC sequences and then golden-gate cloned into the genetic encoded circ-arRNA-expressing vector.

To reduce off-target editing, the nucleotide opposite potential off-target adenosines were deleted and then cloned into the genetic encoded circ-arRNA-expressing vector.

For the dual fluorescence reporter, mCherry and EGFP (the start codon ATG of EGFP was deleted) coding sequences were PCR amplified, digested using BsmBI (ThermoFisher Scientific, ER0452), followed by T4 DNA ligase (NEB, M0202L) mediated ligation with 3×GGGGS linkers. The ligation product was subsequently inserted into the pLenti-CMV-MCS-PURO backbone.

For the constructs expressing genes with pathogenic mutations, full-length coding sequences of TP53 (ordered from Vigenebio) gifts from J. Wang's laboratory, Institute of Pathogen Biology, Chinese Academy of Medical Sciences) were amplified from the constructs encoding the corresponding genes with introduction of G-to-A mutations through mutagenesis PCR. The amplified product was cloned into the pLenti-CMV-MCS-mCherry backbone through the Gibson cloning method.

Production and Purification of circRNA In Vitro

The production of circRNAs is according to methods described in Abe, N. et al. “Preparation of Circular RNA In Vitro,” Circular RNAs. Humana Press, New York, NY, 2018. 181-192; and Chen, H. et al. “Preferential production of RNA rings by T4 RNA ligase 2 without any splint through rational design of precursor strand,” Nucleic Acids Research 48, e54-e54 (2020). Briefly, circRNA precursors were synthesized via in vitro transcriptions (IVT) from the linearized circRNA plasmid templates with HISCRIBE™ T7 High Yield RNA Synthesis Kit (New England Biolabs, #E2040S). After IVT, IVT products were treated with DNase I (New England Biolabs, #M0303S) for 30 min to digest the DNA templates. For T4 Rnl cyclization, T4 Rnl 1 (New England Biolabs, #M0239L) or T4 Rnl 2 (New England Biolabs, #M0204L) was added in the linear circRNA precursors and incubated at 37° C. overnight after DNase I digestion. For group1 autocatalysis cyclization, GTP was added into the reaction at a final concentration of 2 mM after DNase I digestion, and then reactions were incubated at 55° C. for 15 min to catalyze cyclization of circRNAs. Then, cyclized circ-arRNA was column purified with Monarch RNA Cleanup Kit (New England Biolabs, #T2040L). Then, column-purified RNA was heated at 65° C. for 3 min and cooled on ice. Reactions were treated with RNase R (Epicenter, #RNR07250) at 37° C. for 15 min to enrich circRNAs. The RNase R-treated RNA was column purified.

To further enrich circ-arRNA, purified RNase R-treated circ-arRNA was resolved using high-performance liquid chromatography (Agilent HPLC1260) 4.6×300 mm size-exclusion column with particle size of 5 μm and pore size of 2000 Å (Sepax Technologies, #215980P-4630) in RNase-free TE buffer. The circ-arRNA enrich fractions were collected and then column purified (New England Biolabs, #T2040L). To further diminish the immunogenicity of the purified circ-arRNA, circ-arRNA were heated at 65° C. for 3 min, cooled down on ice, and subsequently treated with the quick CIP phosphatase (New England Biolabs, #M0525S). Finally, circ-arRNA were column purified and concentrated with RNA Clean & Concentrator Kit (ZYMO, #R1018).

### Cell Culture and Transfection

HeLa cell lines came from Z. Jiang's laboratory (Peking University) and HEK293T cell line was from C. Zhang's laboratory (Peking University). A549 cell lines were from EdiGene. C2C12 cell lines were purchased from Procell. The MEF cell were generated from Idua-W392X mice. The Hep G2/RPE1/SF268/Cos7/NIH3T3 cell lines were maintained in our laboratory at Peking University. These mammalian cell lines were cultured in Dulbecco's Modified Eagle Medium (Corning, 10-013-CV) with 10% fetal bovine serum (BI), additionally supplemented with 1% penicillin-streptomycin under 5% CO₂ at 37° C.

Plasmids were transfected intro cells with X-tremeGENE HP DNA transfection reagent (Roche, 06366546001) or PEI (Proteintech, B600070) and RNAs cyclized in vitro were transfected into cells with Lipofectamine MessengerMax (Invitrogen, LMRNA003) according to the manufacturer's instructions.

### Cell Line Construction

For the stable reporter cell lines, reporter constructs (pLenti-CMV-MCS-PURO backbone) were cotransfected into HEK293T cells, together with two viral packaging plasmids, pR8.74 and pVSVG. After 72 hours, the supernatant virus was collected and stored at −80° C. HEK293T cells were infected with lentivirus, and then, mCherry-positive cells were sorted via fluorescence-activated cell sorting (FACS) and cultured to select a single clone cell line stably expressing dual fluorescence reporter system without detectable EGFP background. The HEK293T ADAR1^{-/-} and TP53^{-/-} cell lines were generated according to a method described in Zhou, Y., Zhang, H. & Wei, W, “Simultaneous generation of multi-gene knockouts in human cells,” FEBS Letters 11 (2016). ADAR1-targeting single-guide RNA and PCR amplified donor DNA containing CMV-driven puromycin resistant gene were co-transfected into HEK293T cells. Then, cells were treated with puromycin 7 days after transfection. Single clones were isolated from puromycin resistant cells followed by verification through sequencing and Western blot.

### RNA Editing of Endogenous or Exogenous-Expressed Transcripts

For assessing RNA editing on the dual fluorescence reporter, HEK293T-reporter-cells were seeded in 12-well plates (˜1-3×10⁵ cells per well). After 24 hours, cells were transfected with 2 μg linear arRNA or circ-arRNA plasmids. 48 hours after transfection, the editing efficiency was assayed by EGFP⁺ ratio. ADAR1^{-/-} HEK293T cells were transfected with reporter and linear arRNA or circ-arRNA plasmids as in dual fluorescence reporter cells.

To assess RNA editing efficiency in multiple cell lines, 1×10⁵ (HeLa, Hep G2, A549, RPE1, SF268, C2C12, NIH3T3) or 4×10⁵ (HEK293T) cells were seeded in 12-well plates. Twenty-four hours later, reporters and arRNAs plasmid were transfected into these cells. The editing efficiency was assayed by an EGFP⁺ ratio.

To evaluate the EGFP⁺ ratio, cells were sorted and collected by FACS analysis 48 hours post transfection. The mCherry signal was served as a fluorescent selection marker for the reporter/circ-arRNA-expressing cells, and the percentages of EGFP⁺/mCherry⁺ cells were calculated as the readout for editing efficiency.

To assess RNA editing on endogenous mRNA transcripts, HEK293T cells were seeded in six-well plates (8×10⁵ cells per well). Twenty-four hours later, cells were transfected with 3 μg of linear or circular arRNA plasmids. After 48 hours post transfection, cells were sorted and collected by FACS analysis. The editing efficiency was assayed by deep sequencing.

For NGS quantification of the A-to-I editing rate, at 48 hours post transfection, cells were sorted and collected by FACS assay and were subjected to RNA isolation (Zymo, R1055). Then, total RNAs were reverse-transcribed into cDNA via PCR with reverse transcription (RT-PCR) (TIANGEN, KR118), and the targeted locus was PCR amplified with the corresponding primers: mCherry-SpeI-F (SEQ ID NO: 1), mCherry-BsmBI-R1 (SEQ ID NO: 2) and EGFP-BsmBI-F1 (SEQ ID NO: 3). PCR products were purified for Sanger sequencing or NGS (Illumina HiSeq X Ten).

### RNA Editing Analysis for Targeted Sites

For deep sequencing analysis, an index was generated using the targeted site sequence (upstream and downstream 20-nt) of arRNA covering sequences. Reads were aligned and quantified using BWA (v.0.7.10-r789). Alignment BAMs were then sorted by Samtools, and RNA editing sites were analyzed using REDitools (v.1.0.4). The parameters were as follows: -U [AG or TC] -t 8 -n 0.0 -T 6-6 -e -d -u. All significant A-to-G conversion within the arRNA targeting region calculated by Fisher's exact test (P value <0.05) were considered as edits by arRNA. All conversions except for targeted adenosine were off-target edits. Mutations that appeared in control and experimental groups simultaneously were considered to be due to single nucleotide polymorphism.

### Transcriptome-Wide RNA-Sequencing Analysis

Ctrl RNA₁₅₁ or circ-arRNA₁₅₁-PPIA-expressing plasmids with blue fluorescent protein (BFP) expression cassette were transfected into HEK293T cells. BFP⁺ cells were enriched by FACS 48 hours after transfection, and RNAs were purified with RNAprep Pure Micro kit (TIANGEN, DP420). Then, mRNA was purified using NEBNext Poly(A) mRNA Magnetic Isolation Module (New England Biolabs, E7490), processed with NEBNext Ultra II RNA Library Prep Kit for Illumina (New England Biolabs, E7770), and followed by deep sequencing analysis using Illumina HiSeq X Ten platform (2×150-base pair paired end; 30G for each sample). To exclude nonspecific effect caused by transfection, we included the mock group in which we only treated cells with transfection reagent. Each group contained four replications.

The bioinformatics analysis pipeline followed the work by Vogel et al. Quality control of analysis was conducted by using FastQC and quality trim was based on Cutadapt (the first 6-bp for each read were trimmed and up to 20-bp were quality trimmed). AWK scripts were used to filter out the introduced circ-arRNA. After trimming, reads with lengths shorter than 90-nt were filtered out. Subsequently, filtered reads were mapped to the reference genome (GRCh38-hg38) by STAR software. We used GATK Haplotypcaller to call the variants. Raw VCF (variant call format) files generated by GATK were filtered and annotated by GATK VariantFiltration, bcftools, and ANNOVAR. The variants in dbSNP, 1,000 Genome, and EVS were filtered out. The shared variants in six replicates of each group were then selected as the RNA editing sites. The RNA editing level of the mock group was viewed as the background, and the global targets of Ctrl RNA₁₅₁ and circ-arRNA₁₅₁-PPIA were obtained by subtracting the variants in the mock group.

To assess whether circ-arRNA perturbs natural editing homeostasis, we analyzed the global editing sites shared by the Ctrl RNA₁₅₁ group and circ-arRNA₁₅₁-PPIA group. The differential RNA editing rates at native A-to-I editing sites were assessed using Pearson's correlation coefficient analysis.

### Transcriptional Regulatory Activity Assay of p53

TP53^{W53X} cDNA-expressing plasmids and circ-arRNA-expressing plasmids were transfected into HEK293T TP53^{-/-} cells, together with p53-Firefly-luciferase cis-reporting plasmids (YRGene, VXS0446) and Renilla-luciferase plasmids (a gift from Z. Jiang's laboratory, Peking University) for detecting the transcriptional regulatory activity of p53. 48 hours after transfection, cells were collected and assayed with Promega Dual-Glo Luciferase Assay System (Promega, E2940) according to manufacturer's protocol. Luminescence was measured by Infinite M200 reader (TECAN). Fold change of p53-induced luciferase was calculated by the ratio of Firefly luminescence to Renilla luminescence.

### Western Blot

Mouse monoclonal primary antibodies against p53 (Santa Cruz, sc-126) and β-tubulin (CWBiotech, CW0098) were used. HRP-conjugated goat anti-mouse IgG (H+L, 115-035-003) secondary antibody was purchased from Jackson ImmunoResearch. Then, 2×10⁶ cells were sorted to be lysed and an equal amount of protein of each lysate was loaded for SDS-PAGE. Then, sample proteins were transferred onto polyvinylidene difluoride membrane (Bio-Rad Laboratories) and immunoblotted with primary antibodies (anti-p53, 1:300; anti-Tubulin, 1:2000), followed by secondary antibody incubation (1:3,000) and exposure. The experiments were repeated three times. The semi-quantitative analysis was done with Image Lab software.

### Animal Experiments

Idua-W392X mice were ordered from The Jackson Laboratory. All mice were bred and kept under SPF (specific pathogen-free) conditions in the Laboratory Animal Center of Peking University. Animal experiments were approved by Peking University Laboratory Animal Center (Beijing), and undertaken in accordance with National Institute of Health Guide for Care and Use of Laboratory Animals.

The AAV8 of circ-arRNA were packed by PackGene Biotech. AAVs were injected into IDUA-W392X mice by tail vein (B6.129S-Idua^{tm1.1Kmke}/J) at 6-8 weeks of age, at a dose of 1×10¹³ vector genomes per mouse. Mice were monitored four times a week for the duration of the experiment (4-5 weeks).

Harvested mouse tissues were homogenized in 1 mL Trizol and RNA were extracted by Chloroform extraction method. Then, reverse transcribed RNAs of tissues were PCR and analyzed by Sanger sequence or NGS.

### IDUA Catalytic Activity Assay

The gathered cell pellet was resuspended and lysed with 28 μL 0.5% Triton X-100 in 1×PBS buffer and kept on ice for 30 min. Then, 25 μL of the cell lysis was added to 25 μL 190 μM 4-methylumbelliferyl-α-l-iduronidase substrate (Cayman, 2A-19543-500), which was dissolved in 0.4 M sodium formate buffer containing 0.2% Triton X-100 [pH 3.5] and incubated in the dark for 90 min at 37° C. The catalytic reaction was quenched by adding 200 μL 0.5 M NaOH/Glycine buffer [pH 10.3] and then centrifuged for 2 min at 4° C. The supernatant was transferred to a 96-well plate and fluorescence was measured at 365 nm and 450 nm emission wavelength with Infinite M200 reader (TECAN).

### Statistics

An unpaired two-sided Student's t-test was implemented for group comparison. For transcriptome-wide RNA-seq data, DESeq2 (v.1.18.1) was used for analyzing statistical significance. Statistical analyses were performed with R and Prism 8 (GraphPad Software, Inc.).

### Example 1. arRNAs Produced by Pol II Promoter Enable High RNA Editing Efficiency

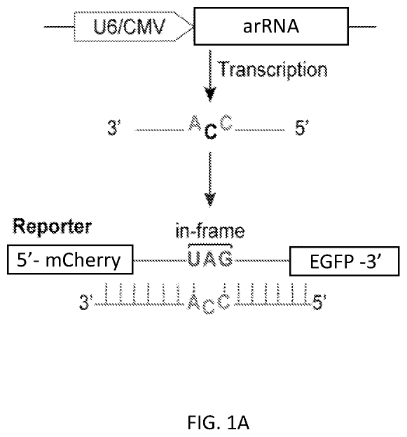

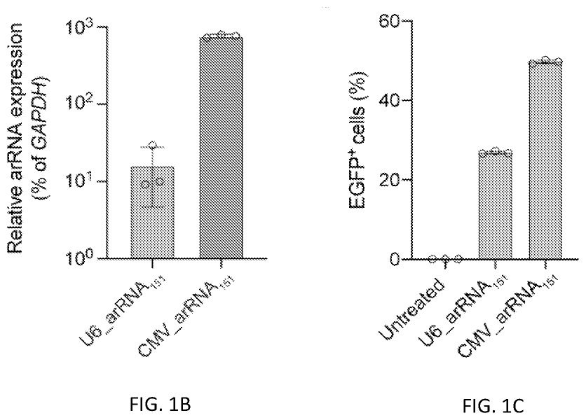

To test whether an RNA Polymerase II (Pol II) can improve editing efficiency, a plasmid expressing arRNA driven by a Pol II promoter (CMV) was constructed. Using a reporter system containing an in-frame stop codon between mCherry and EGFP (Reporter 1, FIG. 1A), the RNA editing efficiency between arRNAs driven by CMV and U6 (Pol III) promoters was compared (arRNA₁₅₁). Untreated cells were used as a mock control (Untreated).

Sequences used are as follows: arRNA₁₅₁ has a sequence of SEQ ID NO: 4. CMV promoter has the nucleic acid sequence of SEQ ID NO: 5. U6 promoter has the nucleic acid sequence of SEQ ID NO: 6. Dual fluorescence reporter-1 comprises sequence of mCherry (SEQ ID NO: 7), sequence comprising 3×GS linker and the targeted A (SEQ ID NO: 8), and sequence of eGFP (SEQ ID NO: 9).

It was found that CMV-arRNA outperforms U6-arRNA in RNA editing (FIGS. 1B-1C). These results suggest that the arRNA abundance is critical for LEAPER efficiency, and the 5′-Cap and 3′-poly(A) do not interfere with arRNAs in targeted editing.

### Example 2. Circular arRNAs Enable Efficient and Long-Lasting Programmable RNA Editing

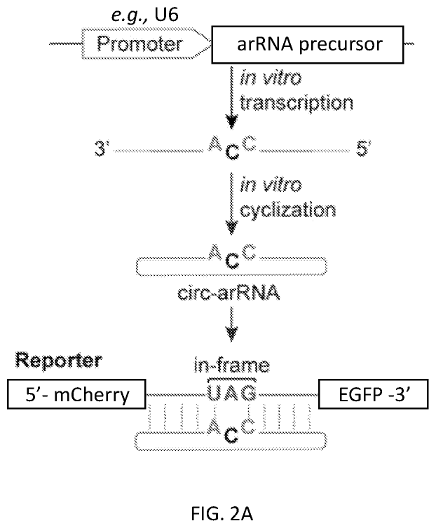

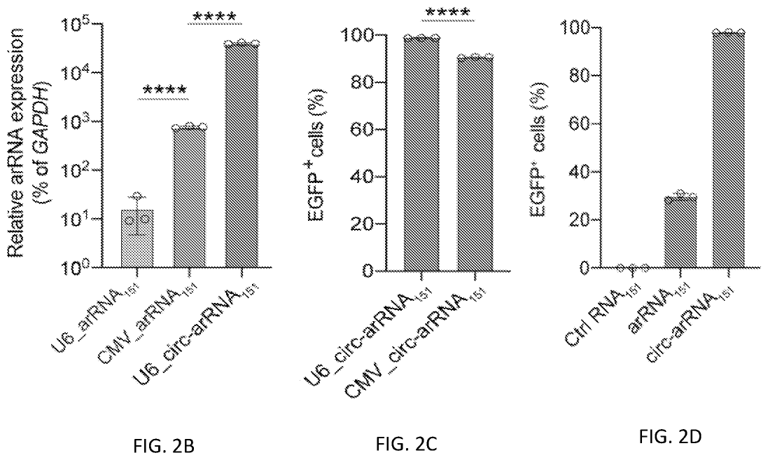

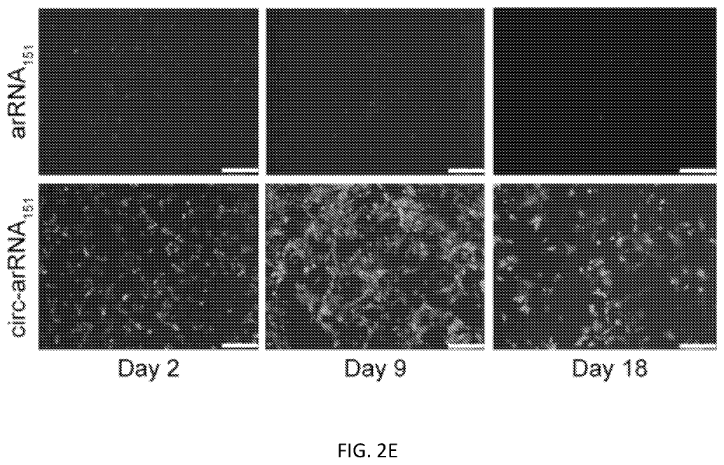

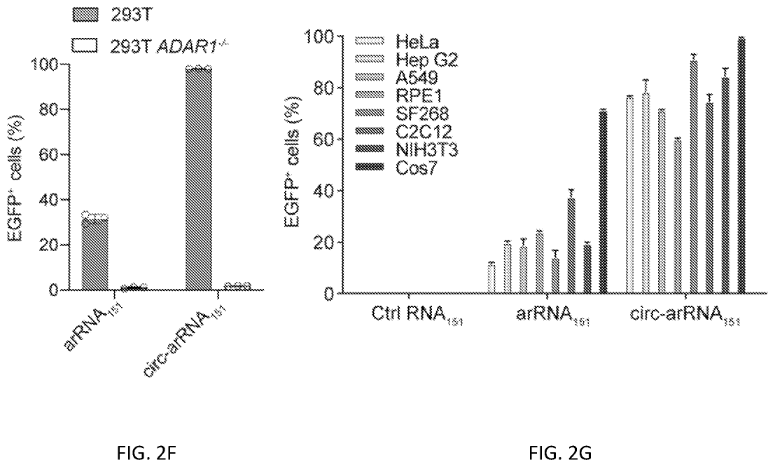

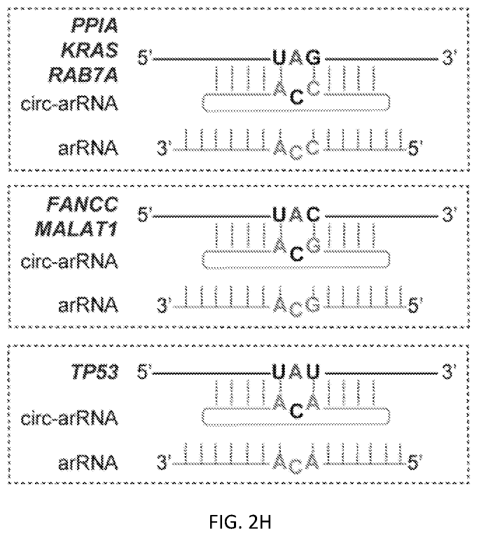

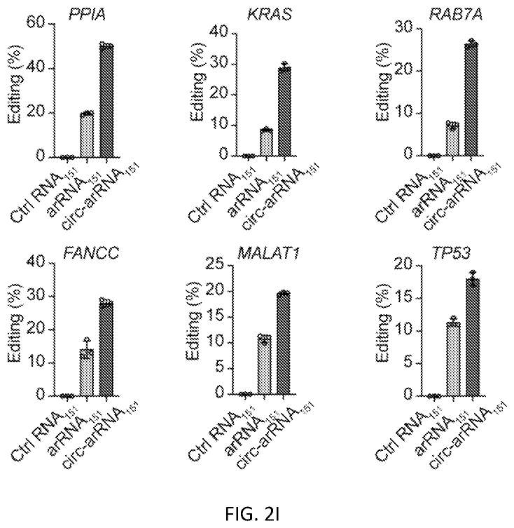

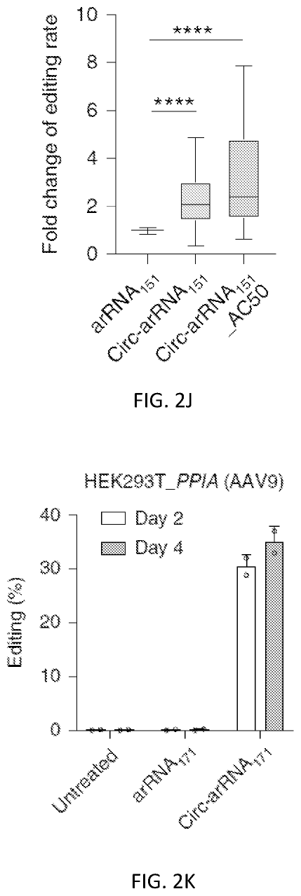

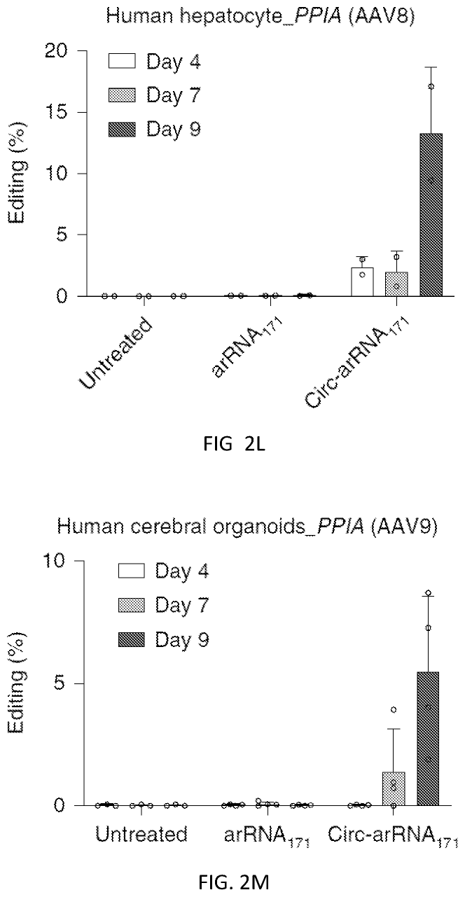

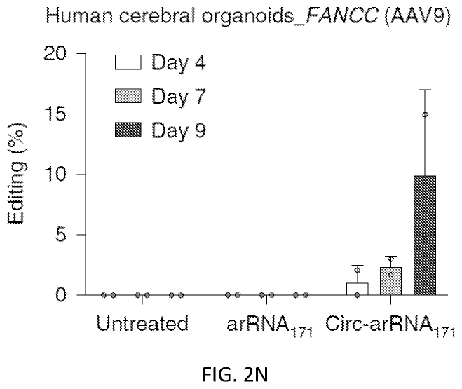

To test whether cyclization of arRNA can improve stability and half-life of RNA, HEK293T cells stably expressing Reporter-1, as described in Example 1, containing an in-frame stop codon between mCherry and EGFP (FIG. 2A) were transfected with plasmids expressing circular arRNA₁₅₁ targeting Reporter-1. The EGFP fluorescence indicates the efficiency of target editing on RNA.

The efficiency of cyclization of arRNA (circ-arRNA) was determined by PCR amplifying the targeted locus with corresponding primer pairs and the PCR products were purified for Sanger sequencing. The Sanger sequence indicated that the circ-arRNAs were successfully generated. Using Reporter 1, RNA editing efficiency between 151-nt arRNAs (arRNA₁₅₁; SEQ ID NO: 4) and circ-arRNAs (circ-arRNA₁₅₁) driven by CMV (Pol II) or U6 (Pol III) promoters was compared. It was found that U6-circ-arRNA₁₅₁ outperforms CMV-arRNA₁₅₁ in RNA editing (FIG. 2B). Circ-arRNA₁₅₁ has a ligation linker sequence of SEQ ID NO: 10 that links the 5′ and 3′ of SEQ ID NO: 4. The 3′ ligation linker sequence is SEQ ID NO: 11 and the 5′ ligation linker sequence is SEQ ID NO: 12. To further increase RNA production, a plasmid expressing circ-arRNA₁₅₁ driven by a Pol II promoter (CMV) was constructed. It was found that U6-circ-arRNA₁₅₁ outperforms CMV-circ-arRNA₁₅₁ in RNA editing, as indicated by EGFP expression in the surrogate reporter assay (FIG. 2C). Based on the above results, it was demonstrated that the editing efficiency for circ-arRNA driven by U6 promoter outperformed that by CMV promoter in RNA editing, and thus U6 promoter was used in all experiments hereinafter.

Using Reporter 1, the RNA editing efficiency between arRNAs (arRNA₁₅₁) and circ-arRNAs (circ-arRNA₁₅₁) driven by U6 promoters was compared. Cells transfected with a non-targeting RNA were used as a control (Ctrl RNA₁₅₁). It was found that circ-arRNA outperforms arRNA, as indicated by the significant increase of EGFP⁺ percentage in transfected HEK293T cells (FIG. 2D). Moreover, such RNA editing was persistent, lasting for at least about 18 days (FIG. 2E).

To test whether RNA editing by circ-arRNAs was also dependent on the endogenous ADAR1 proteins, Reporter-1 and plasmids expressing circular arRNA₁₅₁ targeting Reporter-1 were transfected into HEK293T cells with and without an ADAR1 knockout (HEK293T ADAR^{-/-}). EGFP⁺ percentages were normalized by transfection efficiency, which was determined by mCherry⁺. It was found that RNA editing by both arRNA and circ-arRNAs were dependent on endogenous ADAR, as indicated by complete disappearance in editing-generated EGFP signal in HEK293T ADAR^{-/-} cells (FIG. 2F).

The RNA editing efficiency of arRNAs and circ-arRNAs were further compared in a variety of cell types, including HeLa, HepG2, A549, RPE1, SF268, C2C12, NIH3T3, and Cos7 (FIG. 2G). It was found that circ-arRNA outperformed arRNA in every cell type. To test whether circ-arRNAs could enable effective RNA editing on endogenous transcripts, circ-arRNA₁₅₁ targeting six endogenous transcripts, PPIA (SEQ ID NO: 13), KRAS (SEQ ID NO: 44), RAB7A (SEQ ID NO: 45), FANCC (SEQ ID NO: 46), MALAT1 (SEQ ID NO: 47), and TP53 (SEQ ID NO: 16), were designed. (FIG. 2H). The reverse transcribed RNA was PCR amplified and purified by next-generation sequencing (NGS; Illumina HiSeq X Ten). The NGS results showed that circ-arRNAs outperformed their linear counterparts in targeted RNA editing in all six transcripts (FIG. 2I). Additionally, circ-arRNAs did not show any RNAi effect on the expression of PPIA and FANCC gene transcripts, as indicated by similar relative RNA expression determined by quantitative PCR (data not shown).

Next, to test whether circ-arRNAs could also enable efficient RNA editing on multiple target sites of endogenous transcripts, 151-nt circ-arRNAs were designed to target 20 different RNA sites of nine endogenous genes, PPIB, GUSB, KRAS, MALAT1, TUBB, RAB7A, PPIA, SMYD5 and CTNNB1 (Table 2). NGS analysis revealed that circ-arRNAs outperformed their linear counterparts in targeted RNA editing at 17 of 20 sites. However, circ-arRNAs showed a comparable editing rate on MALAT1 (site 1) and even a decreased editing rate on KRAS (sites 1 and 2). Circ-arRNAs targeting these three sites might have certain structures that interfered with their target recognition or functions to mediate targeted editing activity. To test whether the addition of flexible RNA linkers flanking circ-arRNAs could further optimize their ability in mediating editing activity, fifty-nucleotide flexible polyAC RNA linkers, termed AC50, were added to flanking circ-arRNA₁₅₁, and these circ-arRNA_AC50 linkers gave rise to improved editing rates at 14 sites compared with circ-arRNA₁₅₁. The circ-arRNA_AC50 targeting KRAS (sites 1 and 2) did elevate the editing efficiency of the original circ-arRNAs to a level comparable to that of corresponding linear arRNAs. On average, the editing efficiency of circ-arRNA and circ-arRNA_AC50 was 2.3 and 3.1 fold higher than their linear counterparts, respectively (FIG. 2J).

Adeno-associated virus (AAV) was used to deliver circ-arRNAs into HEK293T cells, human primary hepatocytes and human cerebral organoids. NGS results showed that AAV-delivered circ-arRNAs yielded much higher levels of targeted editing in all these cells and organoids than their linear counterparts, and in a long-lasting fashion (FIG. 2K-N).

TABLE 2
arRNA sequences.

| Name | Sequence (5′ ---> 3′) |
|---|---|
| Ctrl RNA₁₅₁ | acuacaguugcuccgauauuuaggcuacgucaauaggcacuaacuuauug |
| | gcgcuggugaacggacuuccucucgaguaccagaagaugacuacaaaacu |
| | ccuuuccauugcgaguaucggagucuggcucaguuuggccagggaggcac |
| | u |
| arRNA₁₅₁-reporter | ucgccguccagcucgaccaggaugggcaccaccccggugaacagcuccucg |
| | cccuugcucacuggcagagcccuccagcaucgcgagcaggegcugccucc |
| | uccgccgcugccuccuccgccgcugccuccuccgcccugcagcuuguaca |
| circ-arRNA₁₅₁-reporter | cugccaucagucggcguggacuguagaaccaugccgacugauggcag |
| | ucgccguccagcucgaccaggaugggcaccaccccggugaacagcuccucg |
| | cccuugcucacuggcagagcccuccagcaucgegagcaggegcugccucc |
| | uccgccgcugccuccuccgccgcugccuccuccgcccugcagcuuguaca |
| precursor of circ-arRNA₁₅₁- | gccaucagucgccggucccaagcccggauaaaauggggggggaaaccgc |
| reporter (mutation on 5′ | cuaaccaugccgacugauggcagucgccguccagcucgaccaggaugggc |
| Ribozyme) | accaccccggugaacagcuccucgcccuugcucacuggcagagcccuccag |
| | caucgcgagcaggcgcugccuccuccgccgcugccuccuccgccgcugcc |
| | uccuccgcccugcagcuuguacacugccaucagucggcguggacuguaga |
| | acacugccaaugccggucccaagcccggauaaaaguggaggguacagucca |
| | cgc |
| precursor of circ-arRNA₁₅₁- | gccaucagucgccggucccaagcccggauaaaaugggaggggggggaaa |
| reporter (mutation on 3′ | ccgccuaaccaugccgacugauggcagucgccguccagcucgaccaggau |
| Ribozyme) | gggcaccaccccggugaacagcuccucgcccuugcucacuggcagagcccu |
| | ccagcaucgcgagcaggcgcugccuccuccgccgcugccuccuccgccgcu |
| | gccuccuccgcccugcagcuuguacacugccaucagucggcguggacugu |
| | agaacacugccaaugccggucccaagcccggauaaaaguguacaguccacg |
| | c |
| precursor of circ-arRNA₁₅₁- | gccaucagucgccggucccaagcccggauaaaauggggggggaaaccgc |
| reporter (mutation on 5′, 3′ | cuaaccaugccgacugauggcagucgccguccagcucgaccaggaugggc |
| Ribozyme) | accaccccggugaacagcuccucgcccuugcucacuggcagagcccuccag |
| | caucgcgagcaggcgcugccuccuccgccgcugccuccuccgccgcugcc |
| | uccuccgcccugcagcuuguacacugccaucagucggcguggacuguaga |
| | acacugccaaugccggucccaagcccggauaaaaguguacaguccacgc |
| precursor of circ-arRNA₁₅₁- | aaccaugccgacugauggcagucgccguccagcucgaccaggaugggcac |
| reporter (deletion on 5′ | caccccggugaacagcuccucgcccuugcucacuggcagagcccuccagca |
| Ribozyme) | ucgcgagcaggcgcugccuccuccgccgcugccuccuccgccgcugccuc |
| | cuccgcccugcagcuuguacacugccaucagucggcguggacuguagaac |
| | acugccaaugccggucccaagcccggauaaaaguggaggguacaguccac |
| | gc |
| precursor of circ-arRNA₁₅₁- | gccaucagucgccggucccaagcccggauaaaaugggaggggggggaaa |
| reporter (deletion on 3′ | ccgccuaaccaugccgacugauggcagucgccguccagcucgaccaggau |
| Ribozyme) | gggcaccaccccggugaacagcuccucgcccuugcucacuggcagagcccu |
| | ccagcaucgcgagcaggcgcugccuccuccgccgcugccuccuccgccgcu |
| | gccuccuccgcccugcagcuuguacacugccaucagucggcguggacugu |
| | ag |
| precursor of circ-arRNA₁₅₁- | aaccaugccgacugauggcagucgccguccagcucgaccaggaugggcac |
| reporter (deletion on 5′, 3′ | caccccggugaacagcuccucgcccuugcucacuggcagagcccuccagca |
| Ribozyme) | ucgcgagcaggcgcugccuccuccgccgcugccuccuccgccgcugccuc |
| | cuccgcccugcagcuuguacacugccaucagucggcguggacuguag |
| arRNA₁₅₁-PPIB (site1) | uccuguagcuaaggccacaaaauuauccacuguuuuuggaacagucuuuc |
| | cgaagagaccaaagaucacccggcccacaucuucaucuccaauucguaggu |
| | caaaauacaccuugacggugacuuugggccccuucuucuucucaucggcc |
| circ-arRNA₁₅₁-PPIB (site1) | cugccaucagucggcguggacuguagaaccaugccgacugauggcag |
| | uccuguagcuaaggccacaaaauuauccacuguuuuuggaacagucuuuc |
| | cgaagagaccaaagaucacccggcccacaucuucaucuccaauucguaggu |
| | caaaauacaccuugacggugacuuugggccccuucuucuucucaucggcc |
| circ-arRNA₁₅₁_AC50-PPIB | AAAAACAAAAAACAAAAAAAACAAAAAAAAAAC |
| (site1) | CAAAAAAACAAAACACAuccuguagcuaaggccacaaaauua |
| | uccacuguuuuuggaacagucuuuccgaagagaccaaagaucacceggcc |
| | cacaucuucaucuccaauucguaggucaaaauacaccuugacggugacuu |
| | ugggccccuucuucuucucaucggccAAAAAAAAAAACAAA |
| | AAAAACAAAAAAAAAACCAAAAAAACAAAACAC |
| | Acugccaucagucggcguggacuguagaaccaugccgacugauggcag |
| arRNA₁₅₁-PPIB (site2) | gcccuggaucaugaaguccuugauuacacgauggaauuugcuguuuuug |
| | uagccaaauccuuucucuccuguagccaaggccacaaaauuauccacuguu |
| | uuuggaacagucuuuccgaagagaccaaagaucacccggccuacaucuuca |
| circ-arRNA₁₅₁-PPIB (site2) | cugccaucagucggcguggacuguagaaccaugccgacugauggcag |
| | gcccuggaucaugaaguccuugauuacacgauggaauuugcuguuuuug |
| | uagccaaauccuuucucuccuguagccaaggccacaaaauuauccacuguu |
| | uuuggaacagucuuuccgaagagaccaaagaucacccggccuacaucuuca |
| circ-arRNA₁₅₁_AC50-PPIB | AAAAACAAAAAACAAAAAAAACAAAAAAAAAAC |
| (site2) | CAAAAAAACAAAACACAgcccuggaucaugaaguccuugau |
| | uacacgauggaauuugcuguuuuuguagccaaauccuuucucuccuguag |
| | ccaaggccacaaaauuauccacuguuuuuggaacagucuuuccgaagaga |
| | ccaaagaucacccggccuacaucuucaAAAAACAAAAAACAA |
| | AAAAAACAAAAAAAAAACCAAAAAAAAAAACA |
| | CAcugccaucagucggcguggacuguagaaccaugccgacugauggcag |
| arRNA₁₅₁-PPIB (site3) | ugucuuggugcucuccaccuuccgcaccaccuccaugcccucuagaacuu |
| | ugccaaacaccacaugcuugccauccagccaggcugucuugacugucgug |
| | augaagaacugggagccguuggugucuuugccugcguuggccaugcuca |
| | cc |
| circ-arRNA₁₅₁-PPIB (site3) | cugccaucagucggcguggacuguagaaccaugccgacugauggcag |
| | ugucuuggugcucuccaccuuccgcaccaccuccaugcccucuagaacuu |
| | ugccaaacaccacaugcuugccauccagccaggcugucuugacugucgug |
| | augaagaacugggagccguuggugucuuugccugcguuggccaugcuca |
| | cc |
| circ-arRNA₁₅₁_AC50-PPIB | AAAAACAAAAAACAAAAAAAACAAAAAAAAAAC |
| (site3) | CAAAAAAACAAAACACAugucuuggugcucuccaccuuccg |
| | caccaccuccaugcccucuagaacuuugccaaacaccacaugcuugccauc |
| | cagccaggcugucuugacugucgugaugaagaacugggagccguuggug |
| | ucuuugccugcguuggccaugcucaccAAAAAAAAAAACAA |
| | AAAAAAAAAAAAAAAACCAAAAAAAAAAACA |
| | CAcugccaucagucggcguggacuguagaaccaugccgacugauggcag |
| arRNA₁₅₁-PPIB (site4) | gaucacauccuucagggguuuaucccggcugucugucuuggugcucucca |
| | ccuuccgcaccaccuccaugcccuccagaacuuugccaaacaccacaugcu |
| | ugccaucuagccaggcugucuugacugucgugaugaagaacugggagccg |
| circ-arRNA₁₅₁-PPIB (site4) | cugccaucagucggcguggacuguagaaccaugccgacugauggcag |
| | gaucacauccuucagggguuuaucccggcugucugucuuggugcucucca |
| | ccuuccgcaccaccuccaugcccuccagaacuuugccaaacaccacaugcu |
| | ugccaucuagccaggcugucuugacugucgugaugaagaacugggagccg |
| circ-arRNA₁₅₁_AC50-PPIB | AAAAACAAAAAACAAAAAAAACAAAAAAAAAAC |
| (site4) | CAAAAAAACAAAACACAgaucacauccuucagggguuuauc |
| | ccggcugucugucuuggugcucuccaccuuccgcaccaccuccaugcccu |
| | ccagaacuuugccaaacaccacaugcuugccaucuagccaggcugucuuga |
| | cugucgugaugaagaacugggagccgAAAAACAAAAAACAAA |
| | AAAAACAAAAAAAAAACCAAAAAAACAAAACAC |
| | Acugccaucagucggcguggacuguagaaccaugccgacugauggcag |
| arRNA₁₅₁-GUSB (site1) | agccagcagauucuaggugggacgcaggcucguuggccacagaccacauc |
| | acgaccgcggggugguucuuguccccacgcaccacuucuuccaucaccug |
| | cauguggugaugcagagaaacguuguugaagaacugcggcagcgccaggc |
| | c |
| circ-arRNA₁₅₁-GUSB (site1) | cugccaucagucggcguggacuguagaaccaugccgacugauggcag |
| | agccagcagauucuaggugggacgcaggcucguuggccacagaccacauc |
| | acgaccgcggggugguucuuguccccacgcaccacuucuuccaucaccug |
| | cauguggugaugcagagaaacguuguugaagaacugcggcagcgccaggc |
| | c |
| circ-arRNA₁₅₁_AC50-GUSB | AAAAACAAAAAACAAAAAAAACAAAAAAAAAAC |
| (site1) | CAAAAAAACAAAACACAagccagcagauucuaggugggacg |
| | caggcucguuggccacagaccacaucacgaccgcggggugguucuugucc |
| | ccacgcaccacuucuuccaucaccugcauguggugaugcagagaaacguu |
| | guugaagaacugcggcagcgccaggccAAAAACAAAAAACAA |
| | AAAAAACAAAAAAAAAACCAAAAAAACAAAACA |
| | CAcugccaucagucggcguggacuguagaaccaugccgacugauggcag |
| arRNA₁₅₁-GUSB (site2) | aaaggucacaggccgggagggguccaaggauuuggugugagcgaucacca |
| | ucuucaaguaguagccagcagauuccaggugggacgcaggcucguuggcc |
| | acagaccacaucacgaccgcggggugguucuugucccuacgcaccacuuc |
| | u |
| circ-arRNA₁₅₁-GUSB (site2) | cugccaucagucggcguggacuguagaaccaugccgacugauggcagaaa |
| | ggucacaggccgggagggguccaaggauuuggugugagegaucaccaucu |
| | ucaaguaguagccagcagauuccaggugggacgcaggcucguuggccaca |
| | gaccacaucacgaccgcggggugguucuugucccuacgcaccacuucu |
| circ-arRNA₁₅₁-AC50-GUSB | AAAAACAAAAAACAAAAAAAACAAAAAAAAAAC |
| (site2) | CAAAAAAACAAAACACAaaaggucacaggccgggagggguc |
| | caaggauuuggugugagcgaucaccaucuucaaguaguagccagcagauu |
| | ccaggugggacgcaggcucguuggccacagaccacaucacgaccgcgggg |
| | ugguucuugucccuacgcaccacuucuAAAAAAAAAAACAA |
| | AAAAAACAAAAAAAAAACCAAAAAAAAAAACA |
| | CAcugccaucagucggcguggacuguagaaccaugccgacugauggcag |
| arRNA₁₅₁-GUSB (site3) | aucggcaaaauuccaaaugagcucuccaaccacguauuuucugcguuuuu |
| | gauccagacccagaugguacugcuccagcagacuuuucugguacucuuca |
| | gugaacaucagagguggauccuggugaaacccugcaaucguuucugcucc |
| | a |
| circ-arRNA₁₅₁-GUSB (site3) | cugccaucagucggcguggacuguagaaccaugccgacugauggcag |
| | aucggcaaaauuccaaaugagcucuccaaccacguauuuucugcguuuuu |
| | gauccagacccagaugguacugcuccagcagacuuuucugguacucuuca |
| | gugaacaucagagguggauccuggugaaacccugcaaucguuucugcucc |
| | a |
| circ-arRNA₁₅₁-AC50-GUSB | AAAAACAAAAAACAAAAAAAACAAAAAAAAAAC |
| (site3) | CAAAAAAACAAAACACAaucggcaaaauuccaaaugagcucu |
| | ccaaccacguauuuucugcguuuuugauccagacccagaugguacugcuc |
| | cagcagacuuuucugguacucuucagugaacaucagagguggauccuggu |
| | gaaacccugcaaucguuucugcuccaAAAAACAAAAAACAAA |
| | AAAAACAAAAAAAAAACCAAAAAAACAAAACAC |
| | Acugccaucagucggcguggacuguagaaccaugccgacugauggcag |
| arRNA₁₅₁-KRAS (site1) | aucauauucguccacaaaaugauucugaauuagcuguaucgucaaggcac |
| | ucuugccuacgccaccagcuccaaccaccacaaguuuauauucagucauuu |
| | ucagcaggccugggagccgcugagccucuggccccgccgccgccuucagu |
| circ-arRNA₁₅₁-KRAS (site1) | cugccaucagucggcguggacuguagaaccaugccgacugauggcag |
| | aucauauucguccacaaaaugauucugaauuagcuguaucgucaaggcac |
| | ucuugccuacgccaccagcuccaaccaccacaaguuuauauucagucauuu |
| | ucagcaggccugggagccgcugagccucuggccccgccgccgccuucagu |
| circ-arRNA₁₅₁_AC50-KRAS | AAAAACAAAAAACAAAAAAAACAAAAAAAAAAC |
| (site1) | CAAAAAAACAAAACACAaucauauucguccacaaaaugauuc |
| | ugaauuagcuguaucgucaaggcacucuugccuacgccaccagcuccaacc |
| | accacaaguuuauauucagucauuuucagcaggccugggagccgcugagc |
| | cucuggccccgccgccgccuucaguAAAAACAAAAAACAAA |
| | AAAAACAAAAAAAAAACCAAAAAAACAAAACAC |
| | Acugccaucagucggcguggacuguagaaccaugccgacugauggcag |
| arRNA₁₅₁-KRAS (site2) | uugaccugcugugucgagaauauccaagagacagguuucuccaucaauua |
| | cuacuugcuuccuguaggaauccuccauuguuggaucauauucguccaca |
| | aaaugauucugaauuagcuguaucgucaaggcacucuugccuacgccacc |
| | a |
| circ-arRNA₁₅₁-KRAS (site2) | cugccaucagucggcguggacuguagaaccaugccgacugauggcaguug |
| | accugcugugucgagaauauccaagagacagguuucuccaucaauuacua |
| | cuugcuuccuguaggaauccuccauuguuggaucauauucguccacaaaa |
| | ugauucugaauuagcuguaucgucaaggcacucuugccuacgccacca |
| circ-arRNA₁₅₁_AC50-KRAS | AAAAACAAAAAACAAAAAAAACAAAAAAAAAAC |
| (site2) | CAAAAAAACAAAACACAuugaccugcugugucgagaauauc |
| | caagagacagguuucuccaucaauuacuacuugcuuccuguaggaauccu |
| | ccauuguuggaucauauucguccacaaaaugauucugaauuagcuguauc |
| | gucaaggcacucuugccuacgccaccaAAAAACAAAAAACAA |
| | AAAAAACAAAAAAAAAACCAAAAAAACAAAACA |
| | CAcugccaucagucggcguggacuguagaaccaugccgacugauggcag |
| arRNA₁₅₁-KRAS (site3) | gucccucauugcacuguacuccucuugaccugcugugucgagaauaucca |
| | agagacagguuucuccaucaauuaccacuugcuuccuguaggaauccucu |
| | auuguuggaucauauucguccacaaaaugauucugaauuagcuguaucgu |
| | c |
| circ-arRNA₁₅₁-KRAS (site3) | cugccaucagucggcguggacuguagaaccaugccgacugauggcagguc |
| | ccucauugcacuguacuccucuugaccugcugugucgagaauauccaaga |
| | gacagguuucuccaucaauuaccacuugcuuccuguaggaauccucuauu |
| | guuggaucauauucguccacaaaaugauucugaauuagcuguaucguc |
| circ-arRNA₁₅₁_AC50-KRAS | AAAAACAAAAAACAAAAAAAACAAAAAAAAAAC |
| (site3) | CAAAAAAACAAAACACAgucccucauugcacuguacuccucu |
| | ugaccugcugugucgagaauauccaagagacagguuucuccaucaauuac |
| | cacuugcuuccuguaggaauccucuauuguuggaucauauucguccacaa |
| | aaugauucugaauuagcuguaucgucAAAAACAAAAAACAAA |
| | AAAAACAAAAAAAAAACCAAAAAAACAAAACAC |
| | Acugccaucagucggcguggacuguagaaccaugccgacugauggcag |
| arRNA₁₅₁-KRAS (site4) | aaggcaaaucacauuuauuuccuacuaggaccauagguacaucuucagag |
| | uccuuaacucuuuuaauuuguucuccauaauggugaauaucuucaaauga |
| | uuuaguauuauuuauggcaaauacacaaagaaagcccuccccaguccucau |
| circ-arRNA₁₅₁-KRAS (site4) | cugccaucagucggcguggacuguagaaccaugccgacugauggcag |
| | aaggcaaaucacauuuauuuccuacuaggaccauagguacaucuucagag |
| | uccuuaacucuuuuaauuuguucuccauaauggugaauaucuucaaauga |
| | uuuaguauuauuuauggcaaauacacaaagaaagcccuccccaguccucau |
| circ-arRNA₁₅₁_AC50-KRAS | AAAAACAAAAAACAAAAAAAACAAAAAAAAAAC |
| (site4) | CAAAAAAACAAAACACAaaggcaaaucacauuuauuuccuac |
| | uaggaccauagguacaucuucagaguccuuaacucuuuuaauuuguucuc |
| | cauaauggugaauaucuucaaaugauuuaguauuauuuauggcaaauaca |
| | caaagaaagcccuccccaguccucauAAAAACAAAAAACAAA |
| | AAAAACAAAAAAAAAACCAAAAAAACAAAACAC |
| | Acugccaucagucggcguggacuguagaaccaugccgacugauggcag |
| arRNA₁₅₁-KRAS (site5) | uccauaacuucuugcuaaguccugagccuguuuugugucuacuguucua |
| | gaaggcaaaucacauuuauuuccuaccaggaccauagguacaucuucaga |
| | guccuuaacucuuuuaauuuguucucuauaauggugaauaucuucaaaug |
| | au |
| circ-arRNA₁₅₁-KRAS (site5) | cugccaucagucggcguggacuguagaaccaugccgacugauggcagucc |
| | auaacuucuugcuaaguccugagccuguuuugugucuacuguucuagaa |
| | ggcaaaucacauuuauuuccuaccaggaccauagguacaucuucagaguc |
| | cuuaacucuuuuaauuuguucucuauaauggugaauaucuucaaaugau |
| circ-arRNA₁₅₁_AC50-KRAS | AAAAACAAAAAACAAAAAAAACAAAAAAAAAAC |
| (site5) | CAAAAAAACAAAACACAuccauaacuucuugcuaaguccuga |
| | gccuguuuugugucuacuguucuagaaggcaaaucacauuuauuuccuac |
| | caggaccauagguacaucuucagaguccuuaacuvuuuuaauuuguucuc |
| | uauaauggugaauaucuucaaaugauAAAAACAAAAAACAAA |
| | AAAAACAAAAAAAAAACCAAAAAAAAAAACAC |
| | Acugccaucagucggcguggacuguagaaccaugccgacugauggcag |
| arRNA₁₅₁-MALAT1 (site1) | aucaaacaccucacaaaacccccggaacuuuuaaaauaccucuuaaagcac |
| | uucuuguguucucuugagggacagcagguauaguuuaccaccuuuugaa |
| | ggaagauuaaagugugauaguucagggcuuuacuuuccauuacgcaacug |
| | a |
| circ-arRNA₁₅₁-MALAT1 (site1) | cugccaucagucggcguggacuguagaaccaugccgacugauggcagauc |
| | aaacaccucacaaaacccccggaacuuuuaaaauaccucuuaaagcacuuc |
| | uuguguucucuugagggacagcagguauaguuuaccaccuuuugaagga |
| | agauuaaagugugauaguucagggcuuuacuuuccauuacgcaacuga |
| circ-arRNA₁₅₁_AC50-MALAT1 | AAAAACAAAAAACAAAAAAAACAAAAAAAAAAC |
| (site1) | CAAAAAAACAAAACACAaucaaacaccucacaaaacccccgga |
| | acuuuuaaaauaccucuuaaagcacuucuuguguucucuugagggacagc |
| | agguauaguuuaccaccuuuugaaggaagauuaaagugugauaguucagg |
| | gcuuuacuuuccauuacgcaacugaAAAAACAAAAAACAAA |
| | AAAAACAAAAAAAAAACCAAAAAAAAAAACAC |
| | Acugccaucagucggcguggacuguagaaccaugccgacugauggcag |
| arRNA₁₅₁-MALAT1 (site2) | uucuucgguuaaaaauagguucuaguuuuacuuuucuagauuuuucuua |
| | acagcuuauggaacuugaaugcaaaccacacaugcagaaauacuuaaauua |
| | cuagcguguggaaagauuugagcugcaaacuuuagacaaaagaaacaugg |
| | a |
| circ-arRNA₁₅₁-MALAT1 (site2) | cugccaucagucggcguggacuguagaaccaugccgacugauggcaguuc |
| | uucgguuaaaaauagguucuaguuuuacuuuucuagauuuuuuuaaca |
| | gcuuauggaacuugaaugcaaaccacacaugcagaaauacuuaaauuacua |
| | gcguguggaaagauuugagcugcaaacuuuagacaaaagaaacaugga |
| circ-arRNA₁₅₁_AC50-MALAT1 | AAAAACAAAAAACAAAAAAAACAAAAAAAAAAC |
| (site2) | CAAAAAAACAAAACACAuucuucgguuaaaaauagguucua |
| | guuuuacuuuucuagauuuuucuuaacagcuuauggaacuugaaugcaaa |
| | ccacacaugcagaaauacuuaaauuacuagcguguggaaagauuugagcu |
| | gcaaacuuuagacaaaagaaacauggaAAAAACAAAAAACAA |
| | AAAAAACAAAAAAAAAACCAAAAAAACAAAACA |
| | CAcugccaucagucggcguggacuguagaaccaugccgacugauggcag |
| arRNA₁₅₁-TUBB (site1) | ggucagcuucagagugcggaagcagauaucauagagggccucguugucaa |
| | ugcaauaggucucaucaguauucuccaccaacugauggacggagagggug |
| | gcauuguagggcucgaccacggugucagacacuuugggugaaggcaccac |
| | a |
| circ-arRNA₁₅₁-TUBB (site1) | cugccaucagucggcguggacuguagaaccaugccgacugauggcagggu |
| | cagcuucagagugcggaagcagauaucauagagggccucguugucaaugc |
| | aauaggucucaucaguauucuccaccaacugauggacggagaggguggca |
| | uuguagggcucgaccacggugucagacacuuugggugaaggcaccaca |
| circ-arRNA₁₅₁_AC50-TUBB | AAAAACAAAAAACAAAAAAAACAAAAAAAAAAC |
| (site1) | CAAAAAAACAAAACACAggucagcuucagagugcggaagca |
| | gauaucauagagggccucguugucaaugcaauaggucucaucaguauucu |
| | ccaccaacugauggacggagaggguggcauuguagggcucgaccacggug |
| | ucagacacuuugggugaaggcaccacaAAAAACAAAAAACAA |
| | AAAAAACAAAAAAAAAACCAAAAAAACAAAACA |
| | CAcugccaucagucggcguggacuguagaaccaugccgacugauggcag |
| arRNA₁₅₁-TUBB (site2) | accaaauacaaaguugucuggucuaaagaucuggccaaaaggaccugagc |
| | gaacagaguccauggucccagguuccagauccaccaggauggcacgagga |
| | acauauuugccaccuguggcuucauuguaguacacagagaugcgguccag |
| | c |
| circ-arRNA₁₅₁-TUBB (site2) | cugccaucagucggcguggacuguagaaccaugccgacugauggcagacc |
| | aaauacaaaguugucuggucuaaagaucuggccaaaaggaccugagcgaa |
| | cagaguccauggucccagguuccagauccaccaggauggcacgaggaaca |
| | uauuugccaccuguggcuucauuguaguacacagagaugcgguccagc |
| circ-arRNA₁₅₁_AC50-TUBB | AAAAACAAAAAACAAAAAAAACAAAAAAAAAAC |
| (site2) | CAAAAAAACAAAACACAaccaaauacaaaguugucuggucua |
| | aagaucuggccaaaaggaccugagcgaacagaguccauggucccagguuc |
| | cagauccaccaggauggcacgaggaacauauuugccaccuguggcuucau |
| | uguaguacacagagaugcgguccagcAAAAACAAAAAACAAA |
| | AAAAACAAAAAAAAAACCAAAAAAACAAAACAC |
| | Acugccaucagucggcguggacuguagaaccaugccgacugauggcag |
| arRNA₁₅₁-RAB7A | aaaagcuuuuuggcucuuaagucuuugauaaaaggcguacauaauucuug |
| | ugucuacuguacagaauacugccgccagcuggauuucccaauucugagua |
| | acacucugcaauccaaacaggguucaacccuccaccuuacaggccugcauu |
| circ-arRNA₁₅₁-RAB7A | cugccaucagucggcguggacuguagaaccaugccgacugauggcagaaa |
| | agcuuuuuggcucuuaagucuuugauaaaaggcguacauaauucuugug |
| | ucuacuguacagaauacugccgccagcuggauuucccaauucugaguaac |
| | acucugcaauccaaacaggguucaacccuccaccuuacaggccugcauu |
| circ-arRNA₁₅₁_AC50-RAB7A | AAAAACAAAAAACAAAAAAAACAAAAAAAAAAC |
| | CAAAAAAACAAAACACAaaaagcuuuuuggcucuuaagucu |
| | uugauaaaaggcguacauaauucuugugucuacuguacagaauacugccg |
| | ccagcuggauuucccaauucugaguaacacucugcaauccaaacaggguu |
| | caacccuccaccuuacaggccugcauuAAAAAAAAAAACAA |
| | AAAAAACAAAAAAAAAACCAAAAAAACAAAACA |
| | CAcugccaucagucggcguggacuguagaaccaugccgacugauggcag |
| arRNA₁₅₁-PPIA | caguugcugccuacauuuucaauauuccacucaacaaacauugacacuucc |
| | ugggacuggaaaguaaaaaaaauccaaguacuguguagaauuaagcaaaca |
| | agugauguuucagaaguaacccauuacugcuuaaaauaaagccuacauc |
| circ-arRNA₁₅₁-PPIA | cugccaucagucggcguggacuguagaaccaugccgacugauggcagcag |
| | uugcugccuacauuuucaauauuccacucaacaaacauugacacuuccug |
| | ggacuggaaaguaaaaaaaauccaaguacuguguagaauuaagcaaacaag |
| | ugauguuucagaaguaacccauuacugcuuaaaauaaagccuacauc |
| circ-arRNA₁₅₁_AC50-PPIA | AAAAACAAAAAACAAAAAAAACAAAAAAAAAAC |
| | CAAAAAAACAAAACACAcaguugcugccuacauuuucaaua |
| | uuccacucaacaaacauugacacuuccugggacuggaaaguaaaaaaaauc |
| | caaguacuguguagaauuaagcaaacaagugauguuucagaaguaaccca |
| | uuacugcuuaaaauaaagccuacaucAAAAACAAAAAACAAA |
| | AAAAACAAAAAAAAAACCAAAAAAACAAAACAC |
| | Acugccaucagucggcguggacuguagaaccaugccgacugauggcag |
| arRNA₁₅₁-SMYD5 | ccuaaggcaguggucacaggcucgguagcgauaaagugcauuccagagaa |
| | acugugcagccaccaggggccguuccacgaagauggucucccccuuccgg |
| | augagcuguguggcaaacagccccuuucccuuggcgcugcucacgaaacg |
| | g |
| circ-arRNA₁₅₁-SMYD5 | cugccaucagucggcguggacuguagaaccaugccgacugauggcagccu |
| | aaggcaguggucacaggcucgguagegauaaagugcauuccagagaaacu |
| | gugcagccaccaggggccguuccacgaagauggucucccccuuccggaug |
| | agcuguguggcaaacagccccuuucccuuggcgcugcucacgaaacgg |
| circ-arRNA₁₅₁_AC50-SMYD5 | AAAAACAAAAAACAAAAAAAACAAAAAAAAAAC |
| | CAAAAAAACAAAACACAccuaaggcaguggucacaggcucg |
| | guagcgauaaagugcauuccagagaaacugugcagccaccaggggccguu |
| | ccacgaagauggucucccccuuccggaugagcuguguggcaaacagcccc |
| | uuucccuuggcgcugcucacgaaacggAAAAACAAAAAACAA |
| | AAAAAACAAAAAAAAAACCAAAAAAACAAAACA |
| | CAcugccaucagucggcguggacuguagaaccaugccgacugauggcag |
| arRNA₁₅₁-CTNNB1 | acucauacaggacuugggagguauccacauccucuuccucaggauugccu |
| | uuaccacucagagaaggagcuguggcaguggcaccagaauggauuccaga |
| | guccagguaagacuguugcugccagugacuaacagccgcuuuucugucug |
| | g |
| circ-arRNA₁₅₁-CTNNB1 | cugccaucagucggcguggacuguagaaccaugccgacugauggcagacu |
| | cauacaggacuugggagguauccacauccucuuccucaggauugccuuua |
| | ccacucagagaaggagcuguggcaguggcaccagaauggauuccagaguc |
| | cagguaagacuguugcugccagugacuaacagccgcuuuucugucugg |
| circ-arRNA₁₅₁AC50-CTNNB1 | AAAAACAAAAAACAAAAAAAACAAAAAAAAAAC |
| | CAAAAAAACAAAACACAacucauacaggacuugggagguau |
| | ccacauccucuuccucaggauugccuuuaccacucagagaaggagcugug |
| | gcaguggcaccagaauggauuccagaguccagguaagacuguugcugcca |
| | gugacuaacagccgcuuuucugucuggAAAAAAAAAAACAA |
| | AAAAAACAAAAAAAAAACCAAAAAAACAAAACA |
| | CAcugccaucagucggcguggacuguagaaccaugccgacugauggcag |
| arRNA₁₇₁-PPIA | ccaccaugcccaguugcugccuacacuuucaauauuccacucaacaaacau |
| | ugacacuuccugggacuggaaaguaaaaaaaauccaaguacuguguagaa |
| | uuaagcaaacaagugauguuucagaaguaacccauuacugcuuaaaaaaa |
| | gccuacaucaacacucuua |
| circ-arRNA₁₇₁-PPIA | cugccaucagucggcguggacuguagaaccaugccgacugauggcagcca |
| | ccaugcccaguugcugccuacacuuucaauauuccacucaacaaacauuga |
| | cacuuccugggacuggaaaguaaaaaaaauccaaguacuguguagaauua |
| | agcaaacaagugauguuucagaaguaacccauuacugcuuaaaaaaagcc |
| | uacaucaacacucuua |
| arRNA₁₇₁-FANCC | ccucccaucacgggggccguaguagaaggccaagagccacagcagggccg |
| | ugggggguucggcugccgacaucagcaauugcucugccaccaucucagcc |
| | cauccuccgaagugaaugaacaggaaccagcucucaaagggaccuccgcag |
| circ-arRNA₁₇₁-FANCC | cugccaucagucggcguggacuguagaaccaugccgacugauggcagccu |
| | cccaucacgggggccguaguagaaggccaagagccacagcagggccgugg |
| | gggguucggcugccgacaucagcaauugcucugccaccaucucagcccau |
| | ccuccgaagugaaugaacaggaaccagcucucaaagggaccuccgcag |
| arRNA₁₅₁-PPIB | gaggcgcagcauccacaggcggaggcgaaagcagcccggacagcugaggc |
| | cggaagaggguggggccgcgguggccagggagccggcgccgccacgcgcg |
| | ggugggggggacugggguugcucgcgggcuccggggggggggggc |
| | cg |
| circ-arRNA₁₅₁-PPIB | cugccaucagucggcguggacuguagaaccaugccgacugauggcaggag |
| | gcgcagcauccacaggcggaggcgaaagcagcccggacagcugaggccgg |
| | aagaggguggggccgcgguggccagggagccggcgccgccacgcgcgggu |
| | gggggggacugggguugcucgcgggcuccggggggggcgggcgccg |
| arRNA₁₅₁-Iuda^{W392X} | gggugaugggugcuggccaggacacccacuguaugauugcuguccaacac |
| | agccccagccuuugagaccucugcccagaguuguucuccaucuauaagcc |
| | aagcagagggcugaggcuguuggcucucucauaccuagcaucuggcuucc |
| | u |
| In vitro circ-arRNA₁₅₁ AC50- | AAAUCCGUUGACCUUAAACGGUCGUGUGGGUUC |
| Iuda^{W392X} | AAGUCCCUCCACCCCCACGCCGGAAACGCAAUAG |
| | CCGAAAAACAAAAAACAAAAAAAACAAAAAAAA |
| | AACCAAAAAAACAAAACACAgggugaugggugcuggcca |
| | ggacacccacuguaugauugcuguccaacacagccccagccuuugagaccu |
| | cugcccagaguuguucuccaucuauaagccaagcagagggcugaggcugu |
| | uggcucucucauaccuagcaucuggcuuccuACCGGUAAAAAA |
| | CAAAAAACAAAACGGCUAUUAUGCGUUACCGGC |
| | GAGACGCUACGGACU |
| circ-arRNA₁₅₁-PPIA | cugccaucagucggcguggacuguagaaccaugccgacugauggcagcag |
| | uugcugccuacauuuucaauauuccacucaacaaacauugacacuuccug |
| | ggacuggaaaguaaaaaaaauccaaguacuguguagaauuaagcaaacaag |
| | ugauguuucagaaguaacccauuacugcuuaaaauaaagccuacauc |
| arRNA₅₁-reporter | gcccuugcucacuggcagagcccuccagcaucgcgagcaggcgcugccuc |
| | c |
| arRNA_{51ΔC}-reporter | gcccuugcucacuggcagagcccucagcaucgcgagcaggcgcugccucc |
| arRNA₇₁-reporter | acagcuccucgcccuugcucacuggcagagcccuccagcaucgcgagcagg |
| | cgcugccuccuccgccgcug |
| arRNA_{71ΔC}-reporter | acagcuccucgcccuugcucacuggcagagcccucagcaucgcgagcaggc |
| | gcugccuccuccgccgcug |
| arRNA₁₁₁-reporter | gaugggcaccaccccggugaacagcuccucgcccuugcucacuggcagag |
| | cccuccagcaucgcgagcaggcgcugccuccuccgccgcugccuccuccgc |
| | cgcugccucc |
| arRNA_{111ΔC}-reporter | gaugggcaccaccccggugaacagcuccucgcccuugcucacuggcagag |
| | cccucagcaucgcgagcaggcgcugccuccuccgccgcugccuccuccgcc |
| | gcugccucc |
| arRNA_{151ΔC}-reporter | ucgccguccagcucgaccaggaugggcaccaccccggugaacagcuccucg |
| | cccuugcucacuggcagagcccucagcaucgcgagcaggcgcugccuccu |
| | ccgccgcugccuccuccgccgcugccuccuccgcccugcagcuuguaca |
| circarRNA_{151ΔC}-reporter | cugccaucagucggcguggacuguagaaccaugccgacugauggcagucg |
| | ccguccagcucgaccaggaugggcaccaccccggugaacagcuccucgccc |
| | uugcucacuggcagagcccucagcaucgcgagcaggcgcugccuccuccg |
| | ccgcugccuccuccgccgcugccuccuccgcccugcagcuuguaca |
| circ-arRNA_{151-AΔ5}-PPIA | cugccaucagucggcguggacuguagaaccaugccgacugauggcagcag |
| | uugcugccuacauuuucaauauuccacucaacaaacauugacacuuccug |
| | ggacuggaaaguaaaaaaaauccaaguacuguguagaauuaagcaaacaag |
| | gaugucagaagaacccauacugcuuaaaauaaagccuacauc |
| circ-arRNA_{151-AΔ8}-PPIA | cugccaucagucggcguggacuguagaaccaugccgacugauggcagcag |
| | uugcugccuacauuuucaauauuccacucaacaaacauugacacuuccug |
| | ggacuggaaagaaaaaaaauccaaguacuguguagaauaagcaaacaagga |
| | ugucagaagaacccauacugcuaaaauaaagccuacauc |
| circ-arRNA_{151-AΔ14}-AC50-PPIA | AAAAACAAAAAACAAAAAAAACAAAAAAAAAAC |
| | CAAAAAAACAAAACACAcaguugcugccuacauuuucaaau |
| | uccacucaacaaacaugacacuccugggacuggaaagaaaaaaaauccaag |
| | acuguguagaauaagcaaacaaggaugucagaagaacccauacugcuaaaa |
| | aaagccacaucAAAAACAAAAAACAAAAAAAACAAAA |
| | AAAAAACCAAAAAAACAAAACACAcugccaucagucgg |
| | cguggacuguagaaccaugccgacugauggcag |
| In vitro circ-arRNA_{151-AΔ14}- | AAAUCCGUUGACCUUAAACGGUCGUGUGGGUUC |
| AC50-PPIA | AAGUCCCUCCACCCCCACGCCGGAAACGCAAUAG |
| | CCGAAAAACAAAAAACAAAAAAAACAAAAAAAA |
| | AACCAAAAAAACAAAACACAcaguugcugccuacauuuu |
| | caaauuccacucaacaaacaugacacuccugggacuggaaagaaaaaaaau |
| | ccaagacuguguagaauaagcaaacaaggaugucagaagaacccauacugc |
| | uaaaaaaagccacaucACCGGUAAAAAACAAAAAACAAA |
| | ACGGCUAUUAUGCGUUACCGGCGAGACGCUACG |
| | GACU |
| arRNA₃₁-CTNNB1 | gagaaggagcuguggcaguggcaccagaaug |
| circ-arRNA₃₁-CTNNB1 | cugccaucagucggcguggacuguagaaccaugccgacugauggcag |
| | gagaaggagcuguggcaguggcaccagaaug |
| arRNA₇₁-CTNNB1 | uuaccacucagagaaggagcuguggcaguggcaccagaauggauuccaga |
| | g |
| circ-arRNA₇₁-CTNNB1 | cugccaucagucggcguggacuguagaaccaugccgacugauggcaguua |
| | ccacucagagaaggagcuguggcaguggcaccagaauggauuccagag |
| arRNA₁₁₁-CTNNB1 | aggauugccuuuaccacucagagaaggagcuguggcaguggcaccagaau |
| | ggauuccagaguccagguaag |
| circ-arRNA₁₁₁-CTNNB1 | cugccaucagucggcguggacuguagaaccaugccgacugauggcagagg |
| | auugccuuuaccacucagagaaggagcuguggcaguggcaccagaaugga |
| | uuccagaguccagguaag |
| arRNA₁₅₁-CTNNB1 | guauccacauccucuuccucaggauugccuuuaccacucagagaaggagc |
| | uguggcaguggcaccagaauggauuccagaguccagguaagacuguugcu |
| | gccagugacua |
| circ-arRNA₁₅₁-CTNNB1 | cugccaucagucggcguggacuguagaaccaugccgacugauggcaggua |
| | uccacauccucuuccucaggauugccuuuaccacucagagaaggagcugu |
| | ggcaguggcaccagaauggauuccagaguccagguaagacuguugcugcc |
| | agugacua |
| circ-arRNA₁₅₁-p53^{W53X} | cugccaucagucggcguggacuguagaaccaugccgacugauggcaggcu |
| | ggugcaggggccacggggggagcagccucuggcauucugggagcuucau |
| | cuggaccugggucuucagugaaccauuguucaauaucguccggggacagc |
| | aucaaaucauccauugcuugggacggcaagggggacagaacguuguuuu |
| circ-arRNA_{151-AG1}-p53^{W53X} | cugccaucagucggcguggacuguagaaccaugccgacugauggcaggcu |
| | ggugcaggggccacggggggagcagccucuggcauucugggagcuucau |
| | cuggaccugggucuucagugaaccauuguucaagaucguccggggacagc |
| | aucaaaucauccauugcuugggacggcaagggggacagaacguuguuuu |
| circ-arRNA_{151-AG4}-p53^{W53X} | cugccaucagucggcguggacuguagaaccaugccgacugauggcaggcu |
| | ggugcaggggccacggggggagcagccucuggcagucggggagcuucau |
| | cuggaccugggucuucagugaaccauuguucaagaucguccggggacagc |
| | aucaaaucauccagugcuugggacggcaagggggacagaacguuguuuu |
| circ-arRNA_{151-AΔ1}-p53^{W53X} | cugccaucagucggcguggacuguagaaccaugccgacugauggcaggcu |
| | ggugcaggggccacggggggagcagccucuggcauucugggagcuucau |
| | cuggaccugggucuucagugaaccauuguucaaaucguccggggacagca |
| | ucaaaucauccauugcuugggacggcaagggggacagaacguuguuuu |
| circ-arRNA_{151-AΔ4}-p53^{W53X} | cugccaucagucggcguggacuguagaaccaugccgacugauggcaggcu |
| | ggugcaggggccacggggggagcagccucuggcaucgggagcuucaucu |
| | ggaccugggucuucagugaaccauuguucaaaucguccggggacagcauc |
| | aaaucauccaugcuugggacggcaagggggacagaacguuguuuu |
| circ-arRNA₁₅₁_AC50-p53^{W53X} | AAAAACAAAAAACAAAAAAAACAAAAAAAAAAC |
| | CAAAAAAACAAAACACAgcuggugcaggggccacgggggga |
| | gcagccucuggcauucugggagcuucaucuggaccugggucuucagugaa |
| | ccauuguucaauaucguccggggacagcaucaaaucauccauugcuuggg |
| | acggcaagggggacagaacguuguuuuAAAAAAAAAAACAA |
| | AAAAAACAAAAAAAAAACCAAAAAAACAAAACA |
| | CAcugccaucagucggcguggacuguagaaccaugccgacugauggcag |
| circ-arRNA_{151-AΔ4}_AC50- | AAAAACAAAAAACAAAAAAAACAAAAAAAAAAC |
| p53^{W53X} | CAAAAAAACAAAACACAgcuggugcaggggccacgggggga |
| | gcagccucuggcaucgggagcuucaucuggaccugggucuucagugaacc |
| | auuguucaaaucguccggggacagcaucaaaucauccaugcuugggacgg |
| | caagggggacagaacguuguuuuAAAAACAAAAAACAAAA |
| | AAAACAAAAAAAAAACCAAAAAAACAAAACACA |
| | cugccaucagucggcguggacuguagaaccaugccgacugauggcag |
| circ-arRNA₁₅₁-Iuda^{W392X}/mRNA | cugccaucagucggcguggacuguagaaccaugccgacugauggcagggg |
| targeting | ugaugggugcuggccaggacacccacuguaugauugcuguccaacacagc |
| | cccagccuuugagaccucugcccagaguuguucuccauccaacagggcca |
| | ugagccccaugacugugaguacuggcuuucgcagcaacugcacguggg |
| circ-arRNA₁₅₁-Iuda^{W392X}/pre- | cugccaucagucggcguggacuguagaaccaugccgacugauggcagggg |
| mRNA targeting | ugaugggugcuggccaggacacccacuguaugauugcuguccaacacagc |
| | cccagccuuugagaccucugcccagaguuguucuccaucuauaagccaag |
| | cagagggcugaggcuguuggcucucucauaccuagcaucuggcuuccu |
| linker sequence of circ-arRNA | cugccaucagucggcguggacuguagaaccaugccgacugauggcag |
| cyclized by genetically encoded | |
| vector | |
| linker sequence of circ- | AAAAACAAAAAAAAAAAAAACAAAAAAAAAAC |
| arRNA_AC50 cyclized by | CAAAAAAACAAAACACAcugccaucagucggcguggacugu |
| genetically encoded vector | agaaccaugccgacugauggcagAAAAACAAAAAACAAAAA |
| | AAACAAAAAAAAAACCAAAAAAACAAAACACA |

### Example 3. Exploiting In Vitro Cyclization Strategy to Generate Circ-arRNAs for LEAPER

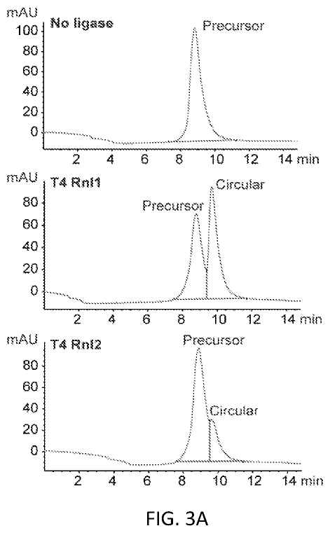

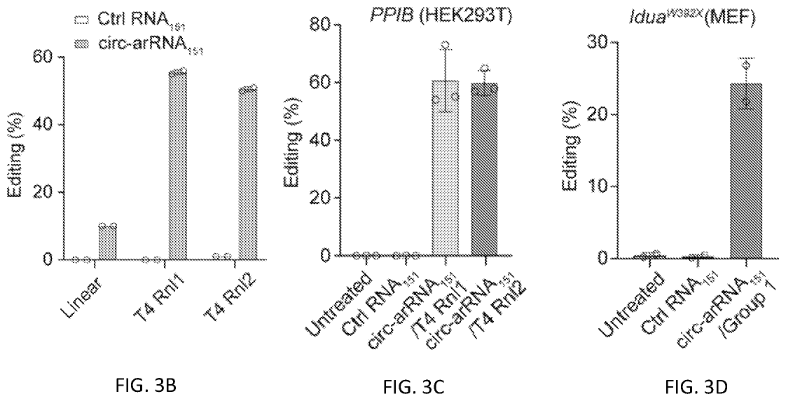

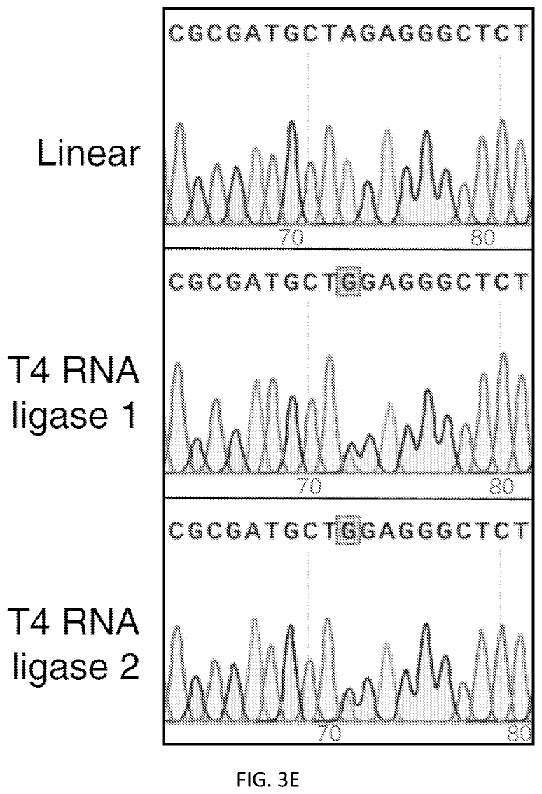

To test an in vitro strategy to generate circ-arRNAs (FIG. 2A), linear arRNAs made from in vitro transcription were cyclized by two types of T4 RNA ligase⁵⁰, T4 Rnl1 and Rnl2, and purified by high-performance liquid chromatography (HPLC) (FIG. 3A). After transfected into HEK293T cells harboring EGFP reporter⁴², EGFP expression was observed at day 1, day 3, and day 7. It was observed that these purified circ-arRNAs could generate much higher and long lasting levels of EGFP signals than the linear version. Additionally, the adenosine to inosine (A-to-I) conversion rates at the targeted site in reporter transcripts for circ-arRNAs were more than 5-fold higher than linear arRNAs for both Sanger sequencing (FIG. 3E) and NGS analysis (FIG. 3B). Moreover, the circ-arRNAs delivered in vitro could also reach over 50% editing rate on endogenous PPIB transcripts (FIG. 3C).

To investigate Group-I ribozyme-mediated autocatalysis activity^{51, 52}, group I ribozyme autocatalysis ligated circ-arRNA were introduced into a primary MEF cell line generated from Hurler syndrome mice. The editing rate on a targeted adenosine of Idua transcripts with a pathogenic point mutation (IDUA^{W392X}) was measured. Untreated cells were used as a mock control (Untreated). Cells transfected with a non-targeting RNA were also used as control (Ctrl RNAs). It was found that NGS results showed that the circ-arRNAs generated by Group-I ribozyme autocatalysis could correct the pathogenic point mutation of IDUA^{W392X} transcripts in the MEF cells, with about 25% editing rate (FIG. 3D). These results showed that the circ-arRNAs, in vivo generated or in vitro produced, could enable efficient and long-lasting targeted RNA editing in endogenous transcripts.

### Example 4. RNA Editing Specificity of Circ-arRNAs

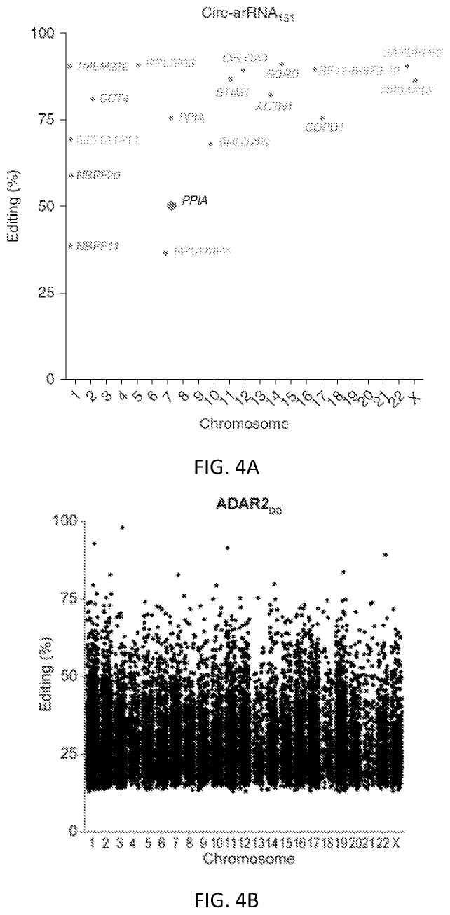

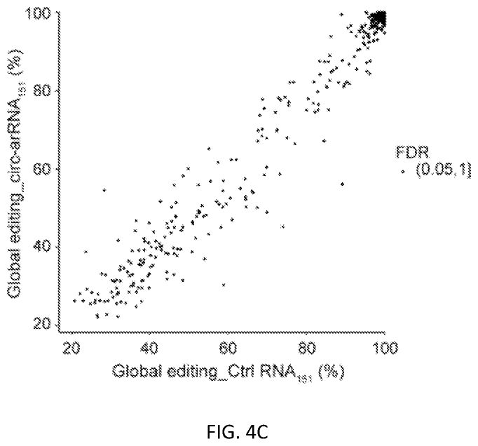

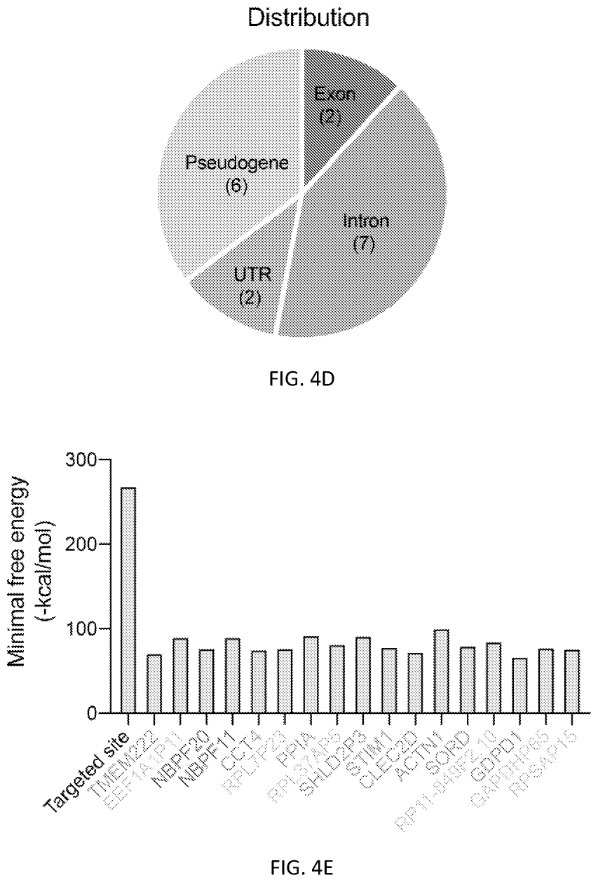

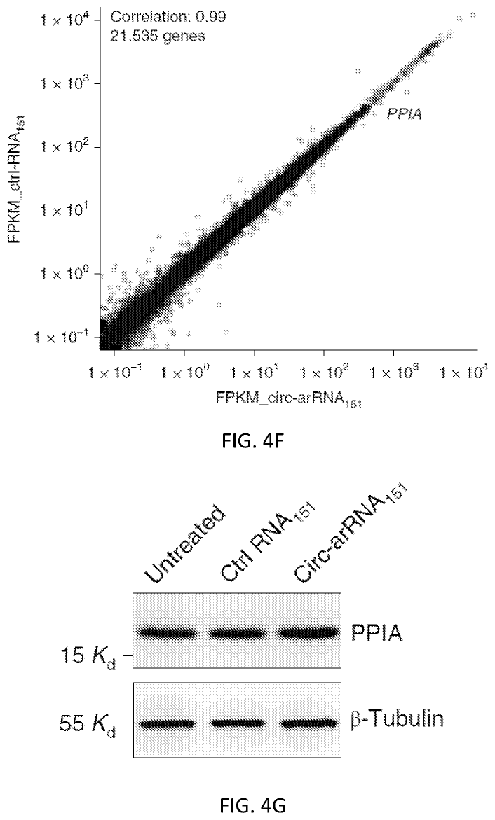

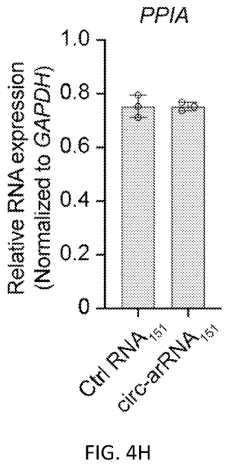

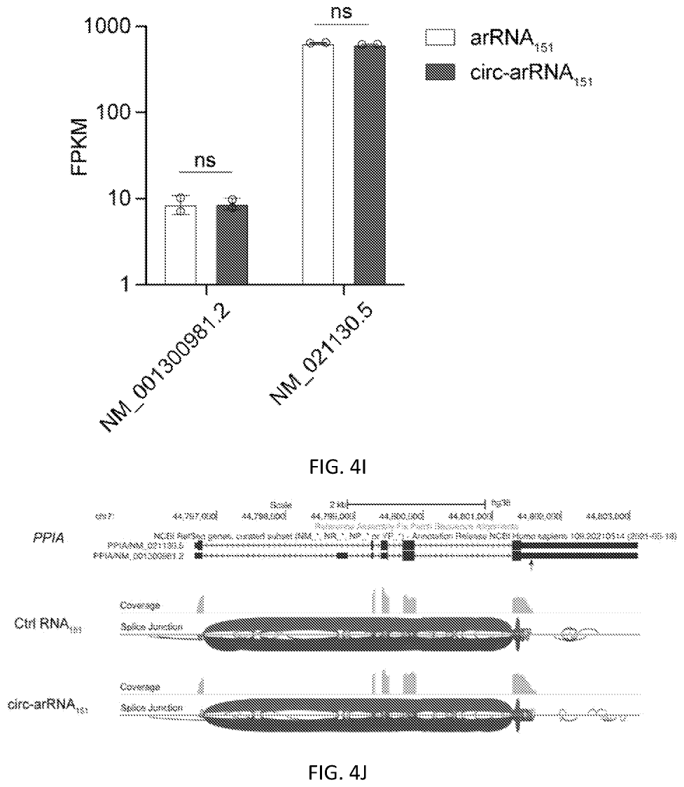

To evaluate the RNA editing specificity of circ-arRNAs, a transcriptome-wide RNA-sequencing analysis was performed. HEK293T cells were transfected with circ-arRNA₁₅₁-PPIA-expressing plasmids. Cells transfected with a non-targeting RNA were used as a control (Ctrl RNAs). The transcriptome-wide RNA-sequencing results showed that there were 17 potential off-target edits in the circ-arRNA₁₅₁-PPIA transfection group (FIG. 4A) and one PPIA on-target site. In contrast, ADAR2 deaminase domain (ADAR2_{DD}) over-expression group, used in many reported RNA editing tools, resulted in nearly 16,588 off-target edits in the RNA transcriptome compared with the control (FIG. 4B). Thus, off-target editing was much higher in the ADAR2_{DD} over-expression group (16,588 off-targets) than the circ-arRNA group (17 off-targets). Most of the 17 identified off-target sites in the circ-arRNA₁₅₁-PPIA transfection group were located in the intron and pseudogene regions (FIG. 4D). Minimum free energy analysis indicated that all these off-target hits failed to form a stable duplex with circ-arRNA151-PPIA (FIG. 4E), and thus are unlikely to be actual sequence-dependent off-targets.

To test whether circ-arRNA₁₅₁-PPIA would affect the expression level of targeted PPIA transcripts, the above transcriptome-wide RNA-seq data was used for further analysis. Circ-arRNA₁₅₁-PPIA mediated editing in PPIA transcripts affected neither the expression nor splicing pattern of PPIA transcripts (FIGS. 4F and 4H-J) and neither was the protein level of PPIA affected (FIG. 4G). Additionally, comparison of A-to-I RNA editing sites shared between the circ-arRNA₁₅₁-PPIA group and Ctrl RNA₁₅₁ group were highly analogous to each other, indicating that circ-arRNAs had little impact on the native A-to-I editing function of endogenous ADAR deaminases (FIG. 4C).

### Example 5. Reducing the Bystander Off-Targets by Engineered Circ-arRNAs

To test bystander off-targets on the arRNA-covered regions of the targeted transcripts, HEK293T cells stably expressing Reporter-1 containing an in-frame stop codon between mCherry and EGFP were transfected with either linear arRNA or circular arRNA targeting Reporter-1 (FIGS. 5A-5B, top panels). Based on the catalytic feature of ADAR1/2 deaminases⁵⁴⁻⁵⁶, it was found that targeted RNA editing was completely eliminated when the nucleotide opposite to the targeted adenosine in linear arRNAs (FIG. 5A, bottom panel) or circ-arRNAs (FIG. 5B, bottom panel) was deleted (arRNA_{ΔC} and circ-arRNA_{ΔC}, respectively).

To reduce bystander off-targets, nucleotides opposite to unwanted adenosines in the circ-arRNA-covered region were deleted (FIG. 5C). Different versions of circ-arRNAs targeting endogenous PPIA transcripts (target sequence of SEQ ID NO: 13) were designed including circ-arRNA₁₅₁-PPIA (with targeting RNA sequence of SEQ ID NO: 14), circ-arRNA_{151-AΔ5}-PPIA (with targeting RNA sequence of SEQ ID NO:139), circ-arRNA_{151-AΔ8}-PPIA (with targeting RNA sequence of SEQ ID NO: 15), circ-arRNA₁₅₁₋AC50-PPIA (with targeting RNA sequence of SEQ ID NO:115) and circ-arRNA_{151-AΔ14_}AC50-PPIA (with targeting RNA sequence of SEQ ID NO: 141) in which uridines on circ-arRNA opposite potential off-target sites were retained or deleted (FIGS. 5C and 5F, and Table 2). In circ-arRNA_{151-AΔ8}, uridines at positions 18, 25, 33, 41, 42, 47, 59, and 87 of SEQ ID NO: 14 were deleted, which correspond to eight potential off-target sites in the target RNA. The 5′ and 3′ ends of the targeting RNA sequence were linked to each other via a ligation linker sequence of SEQ ID NO: 10. NGS results showed that higher on-target editing rate was achieved with circ-arRNA_{151-AΔ8} compared with circ-arRNA₁₅₁ (FIG. 5D), thus the on-target editing rate was not affected by U-deletions on circ-arRNAs. Notably, circ-arRNA_{151-AΔ8} remarkably reduced off-target editing on all eight sites tested (FIG. 5E). These results confirm that, in mammalian cells, adenosines in imperfect double-stranded RNA (dsRNA) with mismatches or bulges can be effectively edited by ADARs with high specificity and efficiency^{57, 58}. In addition, it is found that circ-arRNA_{151-AΔ14}_AC50 almost eliminated all bystander off-targets while still maintaining 60% editing efficiency on the targeted site (FIGS. 5G and 5H). NGS reads surrounding the on-target site showed that circ-arRNA_{151-AΔ14}_AC50 could generate targeted editing with no bystander off-targets in >90% of edited transcripts (FIGS. 5I and 5J).

In vitro-synthesized circ-arRNA_{151-AΔ14} was then tested and it is found that it could also achieve efficient editing in a dose-dependent manner (FIG. 5K). Circ-arRNAs with or without deletion did not affect the expression level of ADAR, nor elicit an innate immune response (FIGS. 5L and 5M).

### Example 6. Recovery of Transcriptional Activity of p53 by Circ-arRNAs with High Efficiency and Specificity

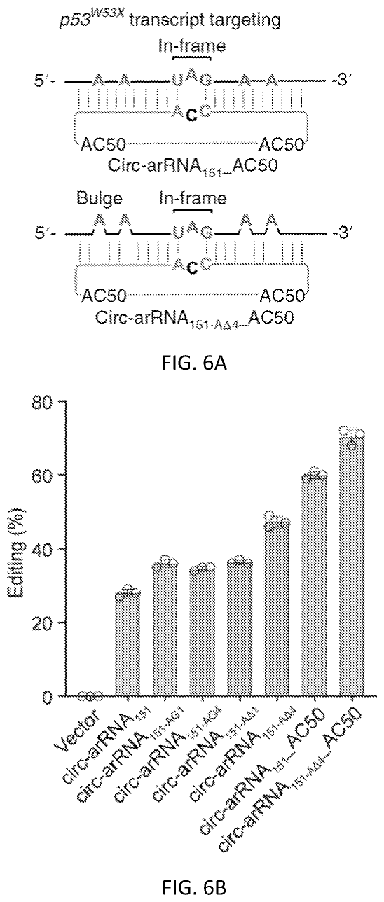

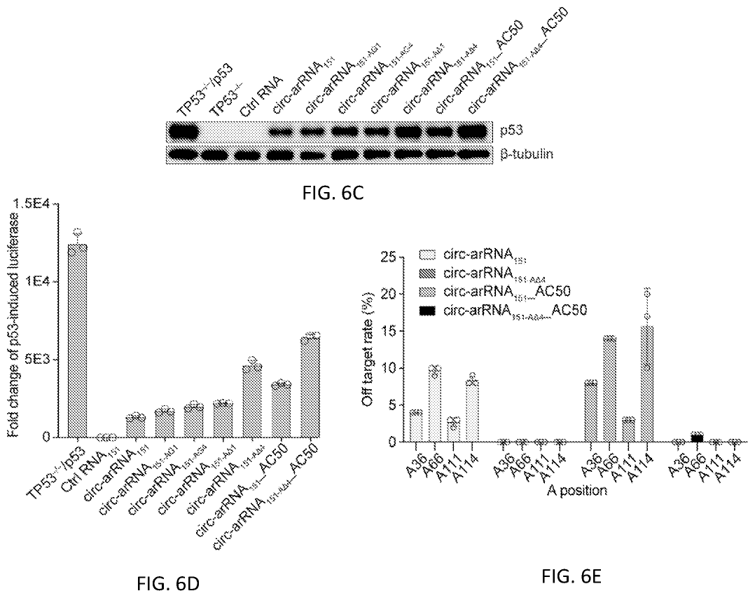

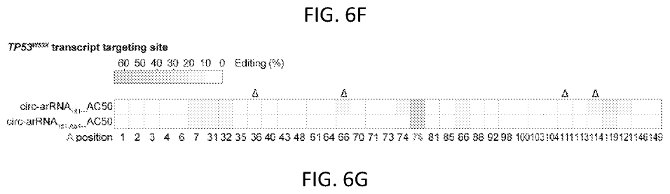

To explore potential therapeutic uses of circ-arRNAs, TP53 tumor suppressor gene, which undergoes frequent mutations in more than 50% of human cancers⁵⁹, was targeted. The c.158G-to-A variant of TP53 is a clinically relevant non-sense mutation (Trp53Ter), generating a non-functional truncated protein (FIG. 6F). Different versions of circ-arRNAs targeting TP53^{W53X}, flanked by flexible RNA linkers and/or harboring U-deletion were designed (FIG. 6A). The TP53^{W53X} transcript has a target sequence of SEQ ID NO: 16. The targeting RNA sequences are as follows: circ-arRNA₁₅₁ (SEQ ID NO: 17), circ-arRNA_{151-AG1} (SEQ ID NO: 18), circ-arRNA_{151-AG4} (SEQ ID NO: 19), circ-arRNA_{151-AΔ1} (SEQ ID NO: 20), circ-arRNA_{151-AΔ4} (SEQ ID NO: 21), Circ-arRNA₁₅₁_AC50 (SEQ ID NO: 17) and circ-arRNA_{151-AΔ4}_AC50 (SEQ ID NO: 21). Circ-arRNA₁₅₁, circ-arRNA_{151-AG1}, circ-arRNA_{151-AG4}, and circ-arRNA_{151-AΔ1}, circ-arRNA_{151-AΔ4} have a ligation linker sequence of SEQ ID NO: 10. In the targeting RNA sequence of circ-arRNA_{151-AG1}, uridine at position 66 of SEQ ID NO: 17 was replaced with G. In the targeting RNA sequence of circ-arRNA_{151-AG4}, uridines at positions 36, 66, 111 and 114 of SEQ ID NO: 17 were replaced with Gs. In the targeting RNA sequence of circ-arRNA_{151-AΔ1}, uridine at position 66 of SEQ ID NO: 17 were deleted. In the targeting RNA sequence of circ-arRNA_{151-AΔ4}, uridines at positions 36, 66, 111 and 114 of SEQ ID NO: 17 were deleted.

NGS analysis showed variable editing rates on the targeted adenosine, ˜30% with circ-arRNA₁₅₁, and ˜40% with circ-arRNA_{151-AG1}, circ-arRNA_{151-AG4}, and circ-arRNA_{151-AΔ1}, with A-G mismatch or U-deletion on one unwanted off-target site (FIG. 6B and Table 2), all higher than the corresponding linear arRNA version⁴². Moreover, circ-arRNA_{151-AΔ4} with U-deletion on four potential off-target sites, conferred a higher editing rate at ˜50% (FIG. 6B).

To test whether addition of flexible RNA linkers flanking the arRNA sequence in circ-arRNA improved its binding ability to targeted RNA resulting in elevated editing efficiency, 50-nt polyAC RNA linkers, termed AC50 (SEQ ID NO: 22), were added to flank arRNA sequences in both circ-arRNA₁₅₁ and circ-arRNA_{151-AΔ4}, giving rise to circ-arRNA₁₅₁_AC50 and circ-arRNA_{151-AΔ4}_AC50. NGS analysis showed that such optimization with flexible linkers boosted the targeted RNA editing efficiency, especially for circ-arRNA_{151-AΔ4}_AC50, which yielded about 70% of editing (FIG. 6B).

In addition to transcript editing, all circ-arRNAs versions could effectively rescue production of full-length p53 protein in HEK293T TP53^{-/-} cells (FIG. 6C). Using a p53-luciferase cis-reporting system previously reported^{60, 61}, it was found that all versions of circ-arRNAs could restore the transcriptional regulation function of p53 (FIG. 6D). Additionally, circ-arRNA_{151-AΔ4}_AC50 had the highest editing rate and restored the highest transcriptional regulation activity when compared to the linear arRNA version⁴² (FIG. 6D).

Potential off-targets in the circ-arRNA-covered region were also examined. It was found that deleting an U nucleotide opposite to the potential off-target A nucleotide on circ-arRNAs almost abolished bystander off-targets on four predicted sites (FIGS. 6E and 6G), while on-target editing rate further increased (FIG. 6B). Although circ-arRNA₁₅₁_AC50 had a higher editing efficiency than circ-arRNA_{151-AΔ4} on targeted site (FIG. 6B), the functional recovery level of circ-arRNA_{151-AΔ4} was much higher than that of circ-arRNA₁₅₁_AC50 because of its low rate of bystander off-target effects (FIGS. 6D and 6G). Collectively, these results showed that the upgraded LEAPER version, termed LEAPER 2.0, could significantly enhance the on-target editing rate while eliminating off-target effects.

### Example 7. Restoration of α-L-Iduronidase (IDUA) Activity in Hurler Syndrome Mouse by Circ-arRNAs

Hurler syndrome is the most severe subtype of Mucopolysaccharidosis type I because of deficiency of α-L-iduronidase (IDUA), a lysosomal metabolic enzyme responsible for metabolism of mucopolysaccharides. To explore the therapeutic potential of circ-arRNAs, the Hurler syndrome mouse model was used. This mouse model harbors a homozygous W392X (TGG-to-TAG) point mutation in the exon 9 of Idua, which is analogous to the W402X mutation found in clinical Hurler syndrome patients.

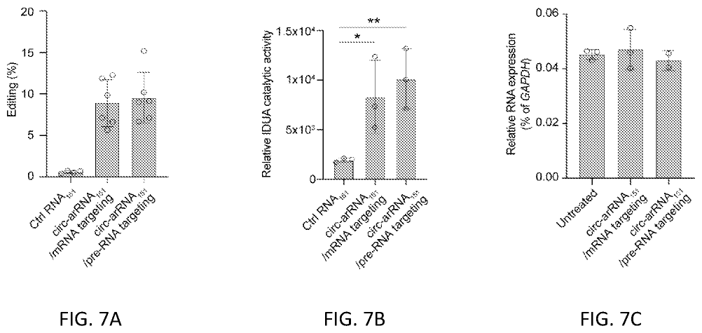

Two versions of circ-arRNAs targeting the mature mRNA or pre-mRNA of Idua, respectively, were designed. The IDUA^{W392X} pre-mRNA transcript has a target sequence of SEQ ID NO: 23. The IDUA^{W392X} mRNA transcript has a target sequence of SEQ ID NO: 25. The circ-arRNA_{mRNA-151} (with targeting RNA sequence of SEQ ID NO: 26) or circ-arRNA_{pre-mRNA-151} (with targeting RNA sequence of SEQ ID NO: 24) were delivered into Idua-W392X mice by the transduction of self-complementary AAV (scAAV) virus. Four weeks later, the mice were sacrificed, and the liver tissues were collected for the measurement of targeted RNA editing and catalytic activity of α-L-iduronidase. NGS analysis revealed that both circ-arRNA₁₅₁/mRNA targeting and circ-arRNA₁₅₁/pre-RNA targeting achieved 10% of targeted editing rate (FIG. 7A). In addition, both circ-arRNAs significantly restored IDUA catalytic activity in liver tissue of Idua-W392X mice (FIG. 7B), without affecting the abundance of Idua transcripts (FIG. 7C). These results demonstrated the therapeutic potential of LEAPER 2.0, with precise, efficient, and lasting targeted RNA editing in certain clinical monogenetic diseases.

### Example 8. Circular arRNAs with Flanking Linker Sequence(s) and/or U Deletions

This example describes the impact of adding flexible linker sequence(s) flanking the arRNA sequence (i.e., flanking linkers) and deleting one or more Us opposite non-target adenosines in a circular RNA on its on-target and off-target editing rates.

### Flanking Linker

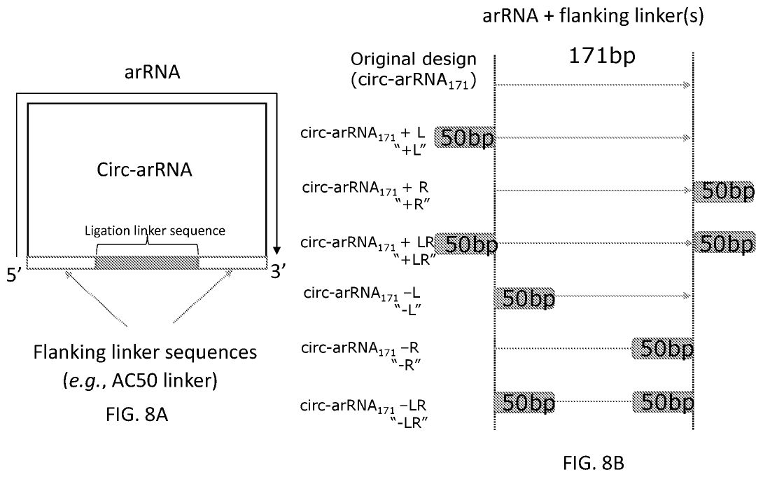

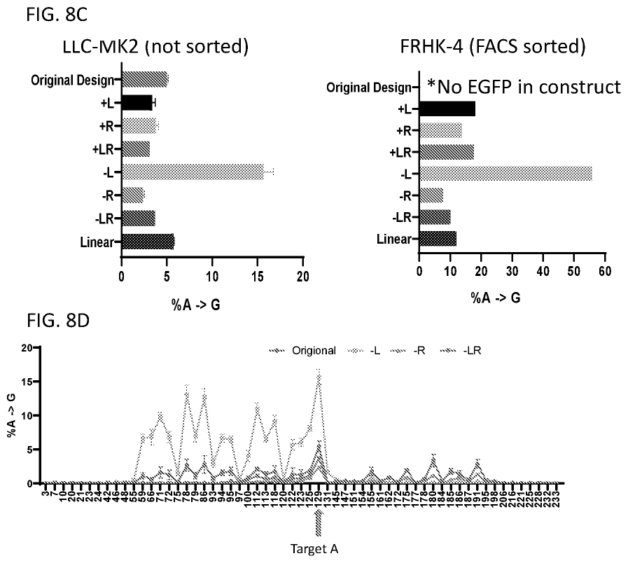

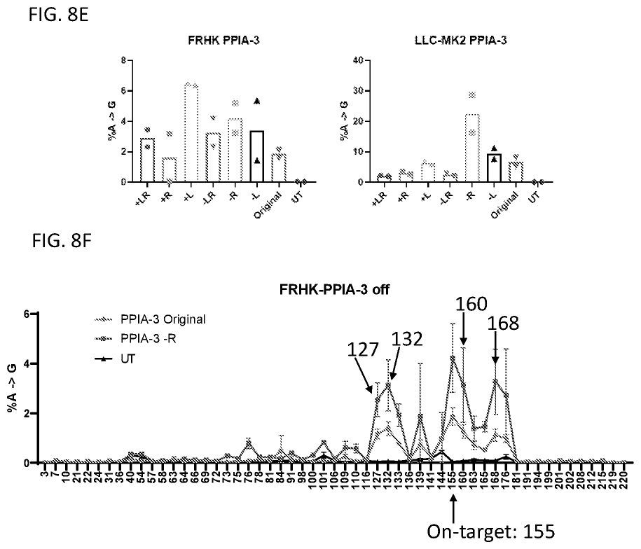

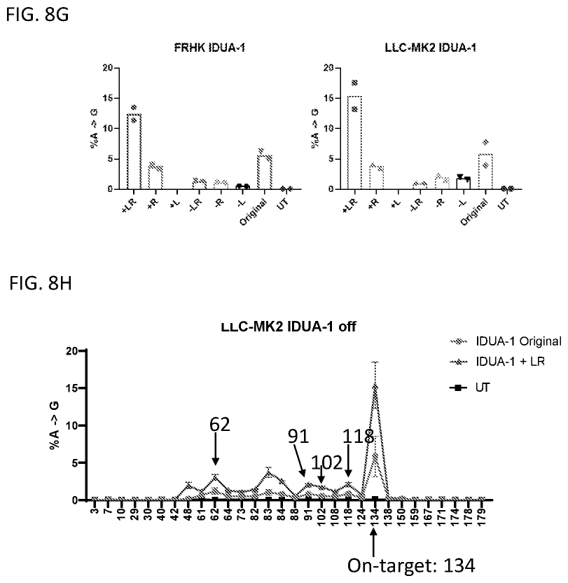

To test whether addition of flexible RNA linkers flanking the arRNA sequence in circ-arRNA could improve its binding ability to targeted RNA resulting in elevated on-target editing efficiency and/or reducing bystander off-target editing effects, 50-nt polyAC RNA linkers, termed AC50 (SEQ ID NO: 22), were added to flank different nucleotide positions at the 5′ or 3′ end of circular 171-nt arRNA (circ-arRNA₁₇₁) sequences. See, FIGS. 8A-8B. In the situations where a stretch of the arRNA sequence has more than five Gs, the middle G in the stretch was replaced with A.

Three target sequences were tested: (1) an A at position 129 in the 3′ UTR of endogenous PPIA transcript (“mf-PPIA-UTR2”; SEQ ID NO: 27); (2) an A at position 155 in the 3′UTR of endogenous PPIA transcript (“mf-PPIA-3”; SEQ ID NO: 28); and (3) an A at position 134 in the 3′ UTR of endogenous IDUA-1 transcript (“mf-IDUA-1”; SEQ ID NO: 29). The reference sequence of Macaca fascicularis can be found with NCBI identifier NC_022274. The reference sequence for Rhesus Ush2a exon is XM_005540847. The targeting RNA sequence of each circ-arRNA₁₇₁ is complementary to the sequence of each target sequence.

Briefly, fetal Rhesus Kidney cells (FRHK-4) and Rhesus Kidney cells (LLC-MK2) were transfected with a recombinant AAV plasmid expressing circular arRNA and eGFP. The rAAV plasmid has AAV2 ITRs flanking an expression cassette, including, from the 5′ to the 3′: U6 promoter operably linked to arRNA, a CAG promoter, EGFP, and WPRE. 48 hours after transfection, RNA samples were obtained from the cells. In FRHK-4 experiments, the cells were subject to FACS sorting based on expression of GFP. Amplicons of the target RNA were obtained, which were subject to NGS analysis.

As shown in FIG. 8C, the various circular arRNAs targeting mf-PPIA-UTR2 resulted in comparable on-target editing efficiency with or without sorting, except for the circ-arRNA having a linker that partially replaced the 5′ sequence (-L) in the arRNA region, which increased on-target editing efficiency. Furthermore, as shown in FIG. 8D, partially replacing the arRNA sequence with an AC50 linker either at the 5′ end (-L) and/or at the 3′ end (-R) diminished bystander off-target editing in the replaced region of the target RNA transcript. Additionally, it was found that on-target editing efficiency as well as off-target editing patterns and efficiency are similar for 171 nt linear and circular arRNA (data not shown).

Depending on the target, different positions of the flanking linker have different effects on the on-target editing efficiency. However, For example, with respect to the mf-PPIA3 target, the circ-arRNA having a linker that partially replaced the 3′ sequence (-R) in the arRNA region consistently led to comparable on-target editing efficiency as the original circ-arRNA₁₇₁ (FIG. 8E). Like mf-PPIA-UTR2, partially replacing the arRNA sequence with an AC50 linker either at the 5′ end (-L) and/or at the 3′ end (-R) diminished bystander off-target editing in the replaced region of the target RNA transcript (FIG. 8F; -L data not shown). A high A to G conversion rate at positions 127, 132, 160 and 168 were observed.

With respect to the mf-IDUA-1 target, circ-arRNAs having a linker that partially replaced either the 5′ sequence (-L) or the 3′ sequence (-R) in the arRNA sequence both reduced on-target editing efficiency. The circ-arRNA having linker sequences flanking both the 5′ end and the 3′ end of the arRNA sequence (RL) had comparable on-target editing efficiency as the original circ-arRNA₁₇₁ (FIG. 8G). The bystander off-target editing effects are shown in FIG. 8H. A high A to G conversion rate at positions 62, 91, 102 and 118 were observed.

Without being bound by any theory, whether including flanking sequences in the circ-arRNA could increase on-target editing efficiency may depend on whether the arRNA could form complex secondary structures. Reduction of off-target editing in the regions of mRNA that correspond to AC50 linker-replaced regions of the arRNA using -L, -R, and -LR circ-arRNAs is consistent with the previous observation that double stranded RNA formation is required for ADAR editing.

### U Deletion

To reduce bystander off-target editing, circ-arRNAs with U opposite certain non-target A were constructed to target mf-PPIA-3 and mf-IDUA-1. Table 1 below lists the positions of the non-target A residues corresponding to the deleted U residue(s) in the circ-arRNAs, and the corresponding circ-arRNA sequences.

TABLE 1
Sequences of circ-arRNAs.

| | Circ-arRNA | Positions of | SEQ ID |
|---|---|---|---|
| Target | name | non-target A | NO |
| mf-PPIA-3 | −1 | 168 | 30 |
| | −2 | 160 | 31 |
| | −3 | 132 | 32 |
| | −4 | 127 | 33 |
| | −43 | 132, 127 | 34 |
| | −432 | 160, 132, 127 | 35 |
| | −4321 | 168, 160, 132, 127 | 36 |
| mf-IDUA-1 | −1 | 118 | 37 |
| | −2 | 102 | 38 |
| | −3 | 91 | 39 |
| | −4 | 62 | 40 |
| | −43 | 91, 62 | 41 |
| | −432 | 102, 91, 62 | 42 |
| | −4321 | 118, 102, 91, 62 | 43 |

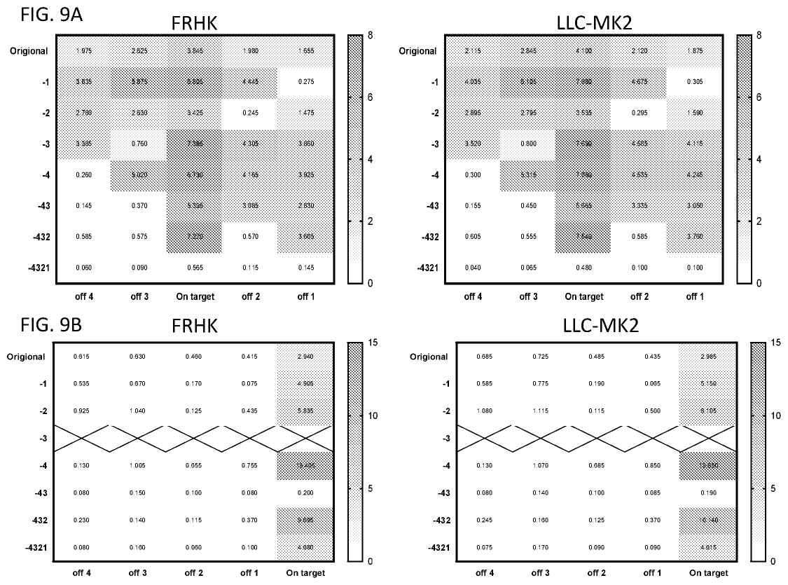

The A to G editing rates at the non-target and target A positions of mf-PPIA-3 and mf-IDUA-1 using the circ-arRNAs are shown in heatmaps in FIG. 9A and FIG. 9B respectively. No data is available for the constructs marked with “X”. With regard to mf-PPIA-3 (FIG. 9A), the 4 off-target As had high in-molecule off-target editing rates. Deletion of the U residue opposite each off-target A residue in the arRNA greatly reduced the off-target editing rates, and in some cases to almost 0. Deletion of U residues opposite multiple off-target A residues in the arRNA could simultaneously reduce the off-target editing rates for the multiple off-target A residues. Deletion of certain U residues or combinations thereof could increase on-target editing rates.

With respect to mf-IDUA-1 (FIG. 9B), the in-molecule off-target rates were generally not high. However, because the target A is close to the end of the IDUA 3′ UTR, the reverse primer may amplify the arRNA, thereby leading to abnormally high on-target rate. The similar off-target editing patterns in both cell types suggest that deletion of U opposite non-target A could increase on-target editing rate for mf-IDUA-1.

### REFERENCES

1. Porteus, M. H. & Carroll, D. Gene targeting using zinc finger nucleases. Nat Biotechnol 23, 967-973 (2005).
2. Boch, J. et al. Breaking the code of DNA binding specificity of TAL-type III effectors. Science 326, 1509-1512 (2009).
3. Moscou, M. J. & Bogdanove, A. J. A simple cipher governs DNA recognition by TAL effectors. Science 326, 1501 (2009).
4. Miller, J. C. et al. A TALE nuclease architecture for efficient genome editing. Nat Biotechnol 29, 143-148 (2011).
5. Jinek, M. et al. A programmable dual-RNA-guided DNA endonuclease in adaptive bacterial immunity. Science 337, 816-821 (2012).
6. Cong, L. et al. Multiplex genome engineering using CRISPR/Cas systems. Science 339, 819-823 (2013).
7. Mali, P. et al. RNA-guided human genome engineering via Cas9. Science 339, 823-826 (2013).
8. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
9. Ma, Y et al. Targeted AID-mediated mutagenesis (TAM) enables efficient genomic diversification in mammalian cells. Nat Methods 13, 1029-1035 (2016).
10. Gaudelli, N. M. et al. Programmable base editing of A*T to G*C in genomic DNA without DNA cleavage. Nature 551, 464-471 (2017).
11. Fry, L. E., Peddle, C. F., Barnard, A. R., McClements, M. E. & MacLaren, R. E. RNA editing as a therapeutic approach for retinal gene therapy requiring long coding sequences. Int J Mol Sci 21 (2020).
12. Tan, M. H. et al. Dynamic landscape and regulation of RNA editing in mammals. Nature 550, 249-254 (2017).
13. Nishikura, K. Functions and regulation of RNA editing by ADAR deaminases. Annu Rev Biochem 79, 321-349 (2010).
14. Bass, B. L. & Weintraub, H. An unwinding activity that covalently modifies its double-stranded RNA substrate. Cell 55, 1089-1098 (1988).
15. Wong, S. K., Sato, S. & Lazinski, D. W. Substrate recognition by ADAR1 and ADAR2. RNA 7, 846-858 (2001).
16. Montiel-Gonzalez, M. F., Vallecillo-Viejo, I., Yudowski, G. A. & Rosenthal, J. J. Correction of mutations within the cystic fibrosis transmembrane conductance regulator by site-directed RNA editing. Proc Natl Acad Sci USA 110, 18285-18290 (2013).
17. Sinnamon, J. R. et al. Site-directed RNA repair of endogenous Mecp2 RNA in neurons. Proc Natl Acad Sci USA 114, E9395-E9402 (2017).
18. Montiel-Gonzalez, M. F., Vallecillo-Viejo, I. C. & Rosenthal, J. J. An efficient system for selectively altering genetic information within mRNAs. Nucleic Acids Res 44, e157 (2016).
19. Hanswillemenke, A., Kuzdere, T., Vogel, P., Jekely, G. & Stafforst, T. Site-Directed RNA Editing in Vivo Can Be Triggered by the Light-Driven Assembly of an Artificial Riboprotein. J Am Chem Soc 137, 15875-15881 (2015).
20. Schneider, M. F., Wettengel, J., Hoffmann, P. C. & Stafforst, T. Optimal guideRNAs for re-directing deaminase activity of hADAR1 and hADAR2 in trans. Nucleic Acids Res 42, e87 (2014).
21. Vogel, P., Hanswillemenke, A. & Stafforst, T. Switching Protein Localization by Site-Directed RNA Editing under Control of Light. ACS Synth Biol 6, 1642-1649 (2017).
22. Vogel, P., Schneider, M. F., Wettengel, J. & Stafforst, T. Improving site-directed RNA editing in vitro and in cell culture by chemical modification of the guideRNA. Angew Chem Int Ed Engl 53, 6267-6271 (2014).
23. Vogel, P. et al. Efficient and precise editing of endogenous transcripts with SNAP-tagged ADARs. Nat Methods 15, 535-538 (2018).
24. Cox, D. B. T. et al. RNA editing with CRISPR-Cas13. Science 358, 1019-1027 (2017).
25. Fukuda, M. et al. Construction of a guide-RNA for site-directed RNA mutagenesis utilising intracellular A-to-I RNA editing. Sci Rep 7, 41478 (2017).
26. Wettengel, J., Reautschnig, P., Geisler, S., Kahle, P. J. & Stafforst, T. Harnessing human ADAR2 for RNA repair—Recoding a PINK1 mutation rescues mitophagy. Nucleic Acids Res 45, 2797-2808 (2017).
27. Heep, M., Mach, P., Reautschnig, P., Wettengel, J. & Stafforst, T. Applying Human ADAR1p110 and ADAR1p150 for Site-Directed RNA Editing-G/C Substitution Stabilizes GuideRNAs against Editing. Genes (Basel) 8 (2017).
28. Katrekar, D. et al. In vivo RNA editing of point mutations via RNA-guided adenosine deaminases. Nat Methods 16, 239-242 (2019).
29. Zhou, C. et al. Off-target RNA mutation induced by DNA base editing and its elimination by mutagenesis. Nature 571, 275-278 (2019).
30. Grunewald, J. et al. Transcriptome-wide off-target RNA editing induced by CRISPR-guided DNA base editors. Nature 569, 433-437 (2019).
31. Grunewald, J. et al. CRISPR DNA base editors with reduced RNA off-target and self-editing activities. Nat Biotechnol 37, 1041-1048 (2019).
32. Jin, S. et al. Cytosine, but not adenine, base editors induce genome-wide off-target mutations in rice. Science 364, 292-295 (2019).
33. Vallecillo-Viejo, I. C., Liscovitch-Brauer, N., Montiel-Gonzalez, M. F., Eisenberg, E. & Rosenthal, J. J. C. Abundant off-target edits from site-directed RNA editing can be reduced by nuclear localization of the editing enzyme. RNA Biol 15, 104-114 (2018).
34. Chew, W. L. et al. A multifunctional AAV-CRISPR-Cas9 and its host response. Nat Methods 13, 868-874 (2016).
35. Wagner, D. L. et al. High prevalence of Streptococcus pyogenes Cas9-reactive T cells within the adult human population. Nat Med 25, 242-248 (2019).
36. Simhadri, V. L. et al. Prevalence of Pre-existing Antibodies to CRISPR-Associated Nuclease Cas9 in the USA Population. Mol Ther Methods Clin Dev 10, 105-112 (2018).
37. Charlesworth, C. T. et al. Identification of preexisting adaptive immunity to Cas9 proteins in humans. Nat Med 25, 249-254 (2019).
38. Teoh, P. J. et al. Aberrant hyperediting of the myeloma transcriptome by ADAR1 confers oncogenicity and is a marker of poor prognosis. Blood 132, 1304-1317 (2018).
39. Haapaniemi, E., Botla, S., Persson, J., Schmierer, B. & Taipale, J. CRISPR-Cas9 genome editing induces a p53-mediated DNA damage response. Nat Med 24, 927-930 (2018).
40. Ihry, R. J. et al. p53 inhibits CRISPR-Cas9 engineering in human pluripotent stem cells. Nat Med 24, 939-946 (2018).
41. Merkle, T. et al. Precise RNA editing by recruiting endogenous ADARs with antisense oligonucleotides. Nat Biotechnol 37, 133-138 (2019).
42. Qu, L. et al. Programmable RNA editing by recruiting endogenous ADAR using engineered RNAs. Nat Biotechnol 37, 1059-1069 (2019).
43. Memczak, S. et al. Circular RNAs are a large class of animal RNAs with regulatory potency. Nature 495, 333-338 (2013).
44. Enuka, Y et al. Circular RNAs are long-lived and display only minimal early alterations in response to a growth factor. Nucleic Acids Res 44, 1370-1383 (2016).
45. Kristensen, L. S. et al. The biogenesis, biology and characterization of circular RNAs. Nat Rev Genet 20, 675-691 (2019).
46. Zhang, X. O. et al. Complementary sequence-mediated exon circularization. Cell 159, 134-147 (2014).
47. Chen, L. L. The biogenesis and emerging roles of circular RNAs. Nat Rev Mol Cell Biol 17, 205-211 (2016).
48. Litke, J. L. & Jaffrey, S. R. Highly efficient expression of circular RNA aptamers in cells using autocatalytic transcripts. Nat Biotechnol 37, 667-675 (2019).
49. Wesselhoeft, R. A. et al. RNA Circularization Diminishes Immunogenicity and Can Extend Translation Duration In Vivo. Mol Cell 74, 508-520 e504 (2019).
50. Beaudry, D. & Perreault, J. P. An efficient strategy for the synthesis of circular RNA molecules. Nucleic Acids Res 23, 3064-3066 (1995).
51. Puttaraju, M. & Been, M. D. Group I permuted intron-exon (PIE) sequences self-splice to produce circular exons. Nucleic Acids Res 20, 5357-5364 (1992).
52. Wesselhoeft, R. A., Kowalski, P. S. & Anderson, D. G. Engineering circular RNA for potent and stable translation in eukaryotic cells. Nat Commun 9, 2629 (2018).
53. Kuttan, A. & Bass, B. L. Mechanistic insights into editing-site specificity of ADARs. Proc Natl Acad Sci USA 109, E3295-3304 (2012).
54. Wahlstedt, H. & Ohman, M. Site-selective versus promiscuous A-to-I editing. Wiley Interdiscip Rev RNA 2, 761-771 (2011).
55. Gallo, A., Vukic, D., Michalik, D., O'Connell, M. A. & Keegan, L. P. ADAR RNA editing in human disease; more to it than meets the I. Hum Genet 136, 1265-1278 (2017).
56. Eggington, J. M., Greene, T. & Bass, B. L. Predicting sites of ADAR editing in double-stranded RNA. Nat Commun 2, 319 (2011).
57. Bazak, L. et al. A-to-I RNA editing occurs at over a hundred million genomic sites, located in a majority of human genes. Genome Res 24, 365-376 (2014).
58. Tian, N. et al. A structural determinant required for RNA editing. Nucleic Acids Res 39, 5669-5681 (2011).
59. Floquet, C., Deforges, J., Rousset, J. P. & Bidou, L. Rescue of non-sense mutated p53 tumor suppressor gene by aminoglycosides. Nucleic Acids Res 39, 3350-3362 (2011).
60. Kern, S. E. et al. Identification of p53 as a sequence-specific DNA-binding protein. Science 252, 1708-1711 (1991).
61. Doubrovin, M. et al. Imaging transcriptional regulation of p53-dependent genes with positron emission tomography in vivo. Proc Natl Acad Sci USA 98, 9300-9305 (2001).
62. Samaridou, E., Heyes, J. & Lutwyche, P. Lipid nanoparticles for nucleic acid delivery: Current perspectives. Adv Drug Deliv Rev 154-155, 37-63 (2020).
63. Bennett, C. F. Therapeutic Antisense Oligonucleotides Are Coming of Age. Annu Rev Med 70, 307-321 (2019).
64. Roberts, T. C., Langer, R. & Wood, M. J. A. Advances in oligonucleotide drug delivery. Nat Rev Drug Discov 19, 673-694 (2020).

SEQUENCE LISTING
mCherry-SpeI-F
SEQ ID NO: 1
TATAACTAGTATGGTGAGCAAGGGCGAGGAG
mCherry-BsmBI-R1
SEQ ID NO: 2
TATACGTCTCATCTACAGATTCTTCCGGCGTGTATACCTTC
EGFP-BsmBI-F1
SEQ ID NO: 3
TATACGTCTCATAGAGATCCCCGGTCGCCACCGTGAGCAAGGGCGAGGAGCTG
arRNA₁₅₁
SEQ ID NO: 4
ACUACAGUUGCUCCGAUAUUUAGGCUACGUCAAUAGGCACUAACUUAUUGGC
GCUGGUGAACGGACUUCCUCUCGAGUACCAGAAGAUGACUACAAAACUCCUUU
CCAUUGCGAGUAUCGGAGUCUGGCUCAGUUUGGCCAGGGAGGCACU
CMV promoter
SEQ ID NO: 5
CGTTACATAACTTACGGTAAATGGCCCGCCTGGCTGACCGCCCAACGACCCCCGC
CCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCA
AGTGTATCATATGCCAAGTACGCCCCCTATTGACGTCAATGACGGTAAATGGCCC
GCCTGGCATTATGCCCAGTACATGACCTTATGGGACTTTCCTACTTGGCAGTACA
TCTACGTATTAGTCATCGCTATTACCATGGTGATGCGGTTTTGGCAGTACATCAAT
GGGCGTGGATAGCGGTTTGACTCACGGGGATTTCCAAGTCTCCACCCCATTGACG
TCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTA
TATAAGCAGAGCT
U6 promoter
SEQ ID NO: 6
GAGGGCCTATTTCCATGATTCCTTCATATTTGCATATACGATACAAGGCTGTTAG
AGAGATAATTAGAATTAATTTGACTGTAAACACAAAGATATTAGTACAAAATAC
GTGACGTAGAAAGTAATAATTTCTTGGGTATTTGCAGTTTTTAAAATTATGTTTTA
AAATGGACTATCATATGCTTACCGTAACTTGAAAGTATTTCGATTTCTTGGCTTTA
TATATCTTGTGGAAAGGAC
mCherry
SEQ ID NO: 7
ATGGTGAGCAAGGGCGAGGAGGATAACATGGCCATCATCAAGGAGTTCATGCGC
TTCAAGGTGCACATGGAGGGCTCCGTGAACGGCCACGAGTTCGAGATCGAGGGC
GAGGGCGAGGGCCGCCCCTACGAGGGCACCCAGACCGCCAAGCTGAAGGTGAC
CAAGGGTGGCCCCCTGCCCTTCGCCTGGGACATCCTGTCCCCTCAGTTCATGTAC
GGCTCCAAGGCCTACGTGAAGCACCCCGCCGACATCCCCGACTACTTGAAGCTGT
CCTTCCCCGAGGGCTTCAAGTGGGAGCGCGTGATGAACTTCGAGGACGGCGGCG
TGGTGACCGTGACCCAGGACTCCTCCCTGCAGGACGGCGAGTTCATCTACAAGGT
GAAGCTGCGCGGCACCAACTTCCCCTCCGACGGCCCCGTAATGCAGAAGAAGAC
CATGGGCTGGGAGGCCTCCTCCGAGCGGATGTACCCCGAGGACGGCGCCCTGAA
GGGCGAGATCAAGCAGAGGCTGAAGCTGAAGGACGGCGGCCACTACGACGCTG
AGGTCAAGACCACCTACAAGGCCAAGAAGCCCGTGCAGCTGCCCGGCGCCTACA
ACGTCAACATCAAGTTGGACATCACCTCCCACAACGAGGACTACACCATCGTGG
AACAGTACGAACGCGCCGAGGGCCGCCACTCCACCGGCGGCATGGACGAGCTGT
ACAAG
3 × GS linker and the targeted adenosine
SEQ ID NO: 8
CTGCAGGGCGGAGGAGGCAGCGGCGGAGGAGGCAGCGGCGGAGGAGGCAGCAG
AAGGTATACACGCCGGAAGAATCTGTAGAGATCCCCGGTCGCCACC
eGFP
SEQ ID NO: 9
GTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGAT
GCCACCTACGGCAAGCTGACCCTGAAGTTCATCTGCACCACCGGCAAGCTGCCC
GTGCCCTGGCCCACCCTCGTGACCACCCTGACCTACGGCGTGCAGTGCTTCAGCC
GCTACCCCGACCACATGAAGCAGCACGACTTCTTCAAGTCCGCCATGCCCGAAG
GCTACGTCCAGGAGCGCACCATCTTCTTCAAGGACGACGGCAACTACAAGACCC
GCGCCGAGGTGAAGTTCGAGGGCGACACCCTGGTGAACCGCATCGAGCTGAAGG
GCATCGACTTCAAGGAGGACGGCAACATCCTGGGGCACAAGCTGGAGTACAACT
ACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACC
ACTACCAGCAGAACACCCCCATCGGCGACGGCCCCGTGCTGCTGCCCGACAACC
ACTACCTGAGCACCCAGTCCGCCCTGAGCAAAGACCCCAACGAGAAGCGCGATC
ACATGGTCCTGCTGGAGTTCGTGACCGCCGCCGGGATCACTCTCGGCATGGACGA
GCTGTACAAGTAA
Ligation linker sequence
SEQ ID NO: 10
CTGCCATCAGTCGGCGTGGACTGTAGAACCATGCCGACTGATGGCAG
3′ Ligation linker sequence
SEQ ID NO: 11
CTGCCATCAGTCGGCGTGGACTGTAG
5′ Ligation linker sequence
SEQ ID NO: 12
AACCATGCCGACTGATGGCAG
PPIA target sequence
SEQ ID NO: 13
GAUGUAGGCUUUAUUUUAAGCAGUAAUGGGUUACUUCUGAAACAUCACUUGU
UUGCUUAAUUCUACACAGUACUUAGAUUUUUUUUACUUUCCAGUCCCAGGAA
GUGUCAAUGUUUGUUGAGUGGAAUAUUGAAAAUGUAGGCAGCAACUG
targeting RNA sequence of PPIA circ-arRNA₁₅₁
SEQ ID NO: 14
CAGUUGCUGCCUACAUUUUCAAUAUUCCACUCAACAAACAUUGACACUUCCUG
GGACUGGAAAGUAAAAAAAAUCCAAGUACUGUGUAGAAUUAAGCAAACAAGU
GAUGUUUCAGAAGUAACCCAUUACUGCUUAAAAUAAAGCCUACAUC
targeting RNA sequence of PPIA circ-arRNA_{151-AΔ8}
SEQ ID NO: 15
CAGUUGCUGCCUACAUUUUCAAUAUUCCACUCAACAAACAUUGACACUUCCUG
GGACUGGAAAGAAAAAAAAUCCAAGUACUGUGUAGAAUAAGCAAACAAGGAU
GUCAGAAGAACCCAUACUGCUAAAAUAAAGCCUACAUC
TP53 target sequence
SEQ ID NO: 16
CUACUUCCUGAAAACAACGUUCUGUCCCCCUUGCCGUCCCAAGCAAUGGAUGA
UUUGAUGCUGUCCCCGGACGAUAUUGAACAAUGGUUCACUGAAGACCCAGGUC
CAGAUGAAGCUCCCAGAAUGCCAGAGGCUGCUCCCCCCGUGGCCC
targeting RNA sequence of TP53 circ-arRNA₁₅₁
SEQ ID NO: 17
GCUGGUGCAGGGGCCACGGGGGGAGCAGCCUCUGGCAUUCUGGGAGCUUCAUC
UGGACCUGGGUCUUCAGUGAACCAUUGUUCAAUAUCGUCCGGGGACAGCAUCA
AAUCAUCCAUUGCUUGGGACGGCAAGGGGGACAGAACGUUGUUUU
targeting RNA sequence of TP53 circ-arRNA_{151-AG1}
SEQ ID NO: 18
GCUGGUGCAGGGGCCACGGGGGGAGCAGCCUCUGGCAUUCUGGGAGCUUCAUC
UGGACCUGGGUCUUCAGUGAACCAUUGUUCAAGAUCGUCCGGGGACAGCAUCA
AAUCAUCCAUUGCUUGGGACGGCAAGGGGGACAGAACGUUGUUUU
targeting RNA sequence of TP53 circ-arRNA_{151-AG4}
SEQ ID NO: 19
GCUGGUGCAGGGGCCACGGGGGGAGCAGCCUCUGGCAGUCGGGGAGCUUCAUC
UGGACCUGGGUCUUCAGUGAACCAUUGUUCAAGAUCGUCCGGGGACAGCAUCA
AAUCAUCCAGUGCUUGGGACGGCAAGGGGGACAGAACGUUGUUUU
targeting RNA sequence of TP53 circ-arRNA_{151-AΔ1}
SEQ ID NO: 20
GCUGGUGCAGGGGCCACGGGGGGAGCAGCCUCUGGCAUUCUGGGAGCUUCAUC
UGGACCUGGGUCUUCAGUGAACCAUUGUUCAAAUCGUCCGGGGACAGCAUCAA
AUCAUCCAUUGCUUGGGACGGCAAGGGGGACAGAACGUUGUUUU
targeting RNA sequence of TP53 circ-arRNA_{151-AΔ4}
SEQ ID NO: 21
GCUGGUGCAGGGGCCACGGGGGGAGCAGCCUCUGGCAUCGGGAGCUUCAUCUG
GACCUGGGUCUUCAGUGAACCAUUGUUCAAAUCGUCCGGGGACAGCAUCAAAU
CAUCCAUGCUUGGGACGGCAAGGGGGACAGAACGUUGUUUU
AC50 linker sequence
SEQ ID NO: 22
AAAAACAAAAAACAAAAAAAACAAAAAAAAAACCAAAAAAAAAAACACA
IDUA pre-mRNA sequence
SEQ ID NO: 23
AGGAAGCCAGAUGCUAGGUAUGAGAGAGCCAACAGCCUCAGCCCUCUGCUUGG
CUUAUAGAUGGAGAACAACUCUAGGCAGAGGUCUCAAAGGCUGGGGCUGUGU
UGGACAGCAAUCAUACAGUGGGUGUCCUGGCCAGCACCCAUCACCC
targeting RNA sequence of circ-arRNA₁₅₁ for IDUA pre-mRNA sequence
(underlined C is opposite the target A)
SEQ ID NO: 24
GGGUGAUGGGUGCUGGCCAGGACACCCACUGUAUGAUUGCUGUCCAACACAGC
CCCAGCCUUUGAGACCUCUGCCCAGAGUUGUUCUCCAUCUAUAAGCCAAGCAG
AGGGCUGAGGCUGUUGGCUCUCUCAUACCUAGCAUCUGGCUUCCU
IDUA mRNA sequence
SEQ ID NO: 25
CCCACGUGCAGUUGCUGCGAAAGCCAGUACUCACAGUCAUGGGGCUCAUGGCC
CUGUUGGAUGGAGAACAACUCUAGGCAGAGGUCUCAAAGGCUGGGGCUGUGU
UGGACAGCAAUCAUACAGUGGGUGUCCUGGCCAGCACCCAUCACCC
targeting RNA sequence of circ-arRNA₁₅₁ for IDUA mRNA sequence
(underlined C is opposite the target A)
SEQ ID NO: 26
GGGUGAUGGGUGCUGGCCAGGACACCCACUGUAUGAUUGCUGUCCAACACAGC
CCCAGCCUUUGAGACCUCUGCCCAGAGUUGUUCUCCAUCUAUAAGCCAAGCAG
AGGGCUGAGGCUGUUGGCUCUCUCAUACCUAGCAUCUGGCUUCCU
mf-PPIA-UTR2
SEQ ID NO: 27
GGCCACCAUGCCCAGCUGCUGCCUACAUUUUCAAUAUUCUACUCAACAAACAU
UGAGACUUCCUGGGUCUGGAAAGAAAGAAAUCCAAGUAUUAUGUAGACUUAA
GCAAACAAGUGAUGUUUCAGAAGUAACCCAUUACUGCUUAAAAUAAAGCCUA
CAUCAACACUCUAA
mf-PPIA-3
SEQ ID NO: 28
CAUGGAACCCAAAGGGAACUGCAGCGAGAGCACAAAGAUUCUAGGAUACUGC
GAGCAAAUGGGGUGGAGGGGUGCUCUCCUGAGCCACAGAAGGAAUGGUCUGG
UGGUUAAGAUAAAACACAAGUCAAACUUAUUCGAGUUGUCCACAGUCAGCAA
UGGUGAUCUUCUUGC
mf-IDUA-1
SEQ ID NO: 29
UAAAAGAAAAUAAUAAAAUAUAAAAAUAUAUUGCAAAGGAGGUGAUGAGAGG
GCAGCGUGGGCACAGUGCAACCCCAGCUCGCCGACCGCCAGUGGAGGUGCAGC
CCAGUGGGGCUCAGCACAGGCUCAUGGAUUUCCAGGGGCUGGAGGCCCUCUUG
GCACAGGGACCUC
mf-PPIA-3 Circ-arRNA-1
SEQ ID NO: 30
CAUGGAACCCAAAGGGAACUGCAGCGAGAGCACAAAGAUUCUAGGAUACUGC
GAGCAAAUGGGGUGGAGGGGGCUCUCCUGAGCCACAGAAGGAAUGGUCUGGU
GGUUAAGAUAAAACACAAGUCAAACUUAUUCGAGUUGUCCACAGUCAGCAAU
GGUGAUCUUCUUGC
mf-PPIA-3 Circ-arRNA-2
SEQ ID NO: 30
CAUGGAACCCAAAGGGAACUGCAGCGAGAGCACAAAGAUUCUAGGAUACUGC
GAGCAAAUGGGGUGGAGGGGUGCUCUCCGAGCCACAGAAGGAAUGGUCUGGU
GGUUAAGAUAAAACACAAGUCAAACUUAUUCGAGUUGUCCACAGUCAGCAAU
GGUGAUCUUCUUGC
mf-PPIA-3 Circ-arRNA-3
SEQ ID NO: 32
CAUGGAACCCAAAGGGAACUGCAGCGAGAGCACAAAGAUUCUAGGAUACUGC
GAGCAAAUGGGGUGGAGGGGUGCUCUCCUGAGCCACAGAAGGAAUGGUCUGG
UGGUAAGAUAAAACACAAGUCAAACUUAUUCGAGUUGUCCACAGUCAGCAAU
GGUGAUCUUCUUGC
mf-PPIA-3 Circ-arRNA-4
SEQ ID NO: 33
CAUGGAACCCAAAGGGAACUGCAGCGAGAGCACAAAGAUUCUAGGAUACUGC
GAGCAAAUGGGGUGGAGGGGUGCUCUCCUGAGCCACAGAAGGAAUGGUCUGG
UGGUUAAGAAAAACACAAGUCAAACUUAUUCGAGUUGUCCACAGUCAGCAAU
GGUGAUCUUCUUGC
mf-PPIA-3 Circ-arRNA-43
SEQ ID NO: 34
CAUGGAACCCAAAGGGAACUGCAGCGAGAGCACAAAGAUUCUAGGAUACUGC
GAGCAAAUGGGGUGGAGGGGUGCUCUCCUGAGCCACAGAAGGAAUGGUCUGG
UGGUAAGAAAAACACAAGUCAAACUUAUUCGAGUUGUCCACAGUCAGCAAUG
GUGAUCUUCUUGC
mf-PPIA-3 Circ-arRNA-432
SEQ ID NO: 35
CAUGGAACCCAAAGGGAACUGCAGCGAGAGCACAAAGAUUCUAGGAUACUGC
GAGCAAAUGGGGUGGAGGGGUGCUCUCCGAGCCACAGAAGGAAUGGUCUGGU
GGUAAGAAAAACACAAGUCAAACUUAUUCGAGUUGUCCACAGUCAGCAAUGG
UGAUCUUCUUGC
mf-PPIA-3 Circ-arRNA-4321
SEQ ID NO: 36
CAUGGAACCCAAAGGGAACUGCAGCGAGAGCACAAAGAUUCUAGGAUACUGC
GAGCAAAUGGGGUGGAGGGGGCUCUCCGAGCCACAGAAGGAAUGGUCUGGUG
GUAAGAAAAACACAAGUCAAACUUAUUCGAGUUGUCCACAGUCAGCAAUGGU
GAUCUUCUUGC
mf-IDUA-1 Circ-arRNA-1
SEQ ID NO: 37
UAAAAGAAAAUAAUAAAAUAUAAAAAUAUAUUGCAAAGGAGGUGAUGAGAGG
GCAGCGUGGGCACAGUGCAACCCCAGCUCGCCGACCGCCAGUGGAGGGCAGCC
CAGUGGGGCUCAGCACAGGCUCAUGGAUUUCCAGGGGCUGGAGGCCCUCUUGG
CACAGGGACCUC
mf-IDUA-1 Circ-arRNA-2
SEQ ID NO: 38
UAAAAGAAAAUAAUAAAAUAUAAAAAUAUAUUGCAAAGGAGGUGAUGAGAGG
GCAGCGUGGGCACAGUGCAACCCCAGCUCGCCGACCGCCAGUGGAGGUGCAGC
CCAGUGGGGCCAGCACAGGCUCAUGGAUUUCCAGGGGCUGGAGGCCCUCUUGG
CACAGGGACCUC
mf-IDUA-1 Circ-arRNA-3
SEQ ID NO: 39
UAAAAGAAAAUAAUAAAAUAUAAAAAUAUAUUGCAAAGGAGGUGAUGAGAGG
GCAGCGUGGGCACAGUGCAACCCCAGCUCGCCGACCGCCAGUGGAGGUGCAGC
CCAGUGGGGCUCAGCACAGGCCAUGGAUUUCCAGGGGCUGGAGGCCCUCUUGG
CACAGGGACCUC
mf-IDUA-1 Circ-arRNA-4
SEQ ID NO: 40
UAAAAGAAAAUAAUAAAAUAUAAAAAUAUAUUGCAAAGGAGGUGAUGAGAGG
GCAGCGUGGGCACAGUGCAACCCCAGCUCGCCGACCGCCAGUGGAGGUGCAGC
CCAGUGGGGCUCAGCACAGGCUCAUGGAUUUCCAGGGGCUGGAGGCCCUCUGG
CACAGGGACCUC
mf-IDUA-1 Circ-arRNA-43
SEQ ID NO: 41
UAAAAGAAAAUAAUAAAAUAUAAAAAUAUAUUGCAAAGGAGGUGAUGAGAGG
GCAGCGUGGGCACAGUGCAACCCCAGCUCGCCGACCGCCAGUGGAGGUGCAGC
CCAGUGGGGCUCAGCACAGGCCAUGGAUUUCCAGGGGCUGGAGGCCCUCUGGC
ACAGGGACCUC
mf-IDUA-1 Circ-arRNA-432
SEQ ID NO: 42
UAAAAGAAAAUAAUAAAAUAUAAAAAUAUAUUGCAAAGGAGGUGAUGAGAGG
GCAGCGUGGGCACAGUGCAACCCCAGCUCGCCGACCGCCAGUGGAGGUGCAGC
CCAGUGGGGCCAGCACAGGCCAUGGAUUUCCAGGGGCUGGAGGCCCUCUGGCA
CAGGGACCUC
mf-IDUA-1 Circ-arRNA-4321
SEQ ID NO: 43
UAAAAGAAAAUAAUAAAAUAUAAAAAUAUAUUGCAAAGGAGGUGAUGAGAGG
GCAGCGUGGGCACAGUGCAACCCCAGCUCGCCGACCGCCAGUGGAGGGCAGCC
CAGUGGGGCCAGCACAGGCCAUGGAUUUCCAGGGGCUGGAGGCCCUCUGGCAC
AGGGACCUC
target sequence of KRAS
SEQ ID NO: 44
ACUGAAGGCGGCGGCGGGGCCAGAGGCUCAGCGGCUCCCAGGCCUGCUGAAAA
UGACUGAAUAUAAACUUGUGGUAGUUGGAGCUGGUGGCGUAGGCAAGAGUGC
CUUGACGAUACAGCUAAUUCAGAAUCAUUUUGUGGACGAAUAUGAU
target sequence of RAB7A
SEQ ID NO: 45
AAUGCAGGCCUGUAAGGUGGAGGGUUGAACCCUGUUUGGAUUGCAGAGUGUU
ACUCAGAAUUGGGAAAUCCAGCUAGCGGCAGUAUUCUGUACAGUAGACACAA
GAAUUAUGUACGCCUUUUAUCAAAGACUUAAGAGCCAAAAAGCUUUU
target sequence of FANCC
SEQ ID NO: 46
CUGCGGAGGUCCCUUUGAGAGCUGGUUCCUGUUCAUUCACUUCGGAGGAUGG
GCUGAGAUGGUGGCAGAGCAAUUACUGAUGUCGGCAGCCGAACCCCCCACGGC
CCUGCUGUGGCUCUUGGCCUUCUACUACGGCCCCCGUGAUGGGAGG
target sequence of MALAT1
SEQ ID NO: 47
UCAGUUGCGUAAUGGAAAGUAAAGCCCUGAACUAUCACACUUUAAUCUUCCU
UCAAAAGGUGGUAAACUAUACCUACUGUCCCUCAAGAGAACACAAGAAGUGC
UUUAAGAGGUAUUUUAAAAGUUCCGGGGGUUUUGUGAGGUGUUUGAU

1: A method for editing a target adenosine in a target RNA in a host cell, comprising introducing a deaminase-recruiting RNA (dRNA) or a construct comprising a nucleic acid sequence encoding the dRNA into the host cell, wherein:
(1) the dRNA comprises a targeting RNA sequence that is capable of hybridizing to the target RNA to form a duplex RNA, wherein the duplex RNA comprises a bulge comprising a non-target adenosine in the target RNA; and
(2) the dRNA is capable of recruiting an adenosine deaminase acting on RNA (ADAR).

2: The method of claim 1, wherein the duplex RNA comprises a bulge at each non-target adenosine in the target RNA.

3: The method of claim 1, wherein the targeting RNA sequence is complementary to the target RNA except for lacking one or more nucleotides opposite to non-target adenosines in the target RNA.

4. (canceled)

5: The method of claim 1, wherein the method has reduced level of editing of the non-target adenosine in the target RNA compared to a method using a dRNA or a construct thereof comprising a targeting RNA sequence that has a nucleotide opposite the non-target adenosine in the target RNA.

6: The method of claim 1, wherein the dRNA is a linear RNA.

7: The method of claim 6, wherein the dRNA is capable of forming a circular RNA.

8: The method of claim 1, wherein the dRNA is a circular RNA.

9: The method of claim 1, wherein the dRNA comprises a linker nucleic acid sequence flanking an end of the targeting RNA sequence, wherein the linker nucleic acid sequence does not substantially form any secondary structure with any part of the dRNA.

10: The method of claim 1, wherein the dRNA comprises a linker nucleic acid sequence replacing an end of the targeting RNA sequence, wherein the linker nucleic acid sequence does not substantially form any secondary structure with any part of the dRNA.

11: A method for editing a target adenosine in a target RNA in a host cell, comprising introducing a dRNA or a construct comprising a nucleic acid sequence encoding the dRNA into the host cell, wherein:
(1) the dRNA comprises a targeting RNA sequence that is capable of hybridizing to the target RNA, wherein the dRNA comprises a linker nucleic acid sequence flanking an end of the targeting RNA sequence, wherein the linker nucleic acid sequence does not substantially form any secondary structure with any part of the dRNA;
(2) the dRNA is capable of recruiting an ADAR; and
(3) the dRNA is a circular RNA or a linear RNA capable of forming a circular RNA.

12: The method of claim 11, wherein the dRNA is a circular RNA.

13: The method of claim 11, wherein the dRNA is a linear RNA capable of forming a circular RNA.

14: The method of claim 11, wherein the linker nucleic acid sequence is about 5 nucleotides (nt) to about 500 nt long.

15: The method of claim 14, wherein the linker nucleic acid sequence is about 50 nt to about 500 nt long.

16-17. (canceled)

18: The method of claim 11, wherein at least about 50% of the linker nucleic acid sequence comprises adenosine or cytidine; optionally wherein 100% of the linker nucleic acid sequence comprises adenosine or cytidine.

19-28. (canceled)

29: The method of claim 11, wherein the dRNA is a circular RNA, and wherein the linker nucleic acid sequence connects the 5′ end of the targeting RNA sequence and the 3′ end of the targeting RNA sequence.

30-62. (canceled)

63: A method for treating or preventing a disease or condition in an individual, comprising editing a target RNA associated with the disease or condition in a cell of the individual according to the method of claim 1.

64-68. (canceled)

69: A dRNA for editing a target RNA comprising a targeting RNA sequence that is capable of hybridizing to the target RNA, wherein the dRNA comprises a linker nucleic acid sequence flanking an end of the targeting RNA sequence, wherein the linker nucleic acid sequence does not substantially form any secondary structure with any part of the dRNA, and wherein the dRNA is a circular RNA or a linear RNA capable of forming a circular RNA.

70-73. (canceled)

74: A method for treating or preventing a disease or condition in an individual, comprising editing a target RNA associated with the disease or condition in a cell of the individual according to the method of claim 11.
